(19) United States
(12) Patent Application Publication
(10) Pub. No.: US 2026/0185068 A1
(43) Pub. Date: Jul. 2, 2026

(54) HIGH FIDELITY NUCLEOTIDE POLYMERASE CHIMERIC PRIME EDITOR SYSTEMS

(71) Applicant: UNIVESITY OF MASSACHUSETTS, Westborough, MA (US)

(72) Inventors: Erik Sontheimer, Auburndale, MA (US); Wen Xue, Natick, MA (US); Bin Liu, Worcester, MA (US); Xiaolong Dong, Worcester, MA (US)

(21) Appl. No.: 18/867,771

(22) PCT Filed: Jun. 1, 2023
(86) PCT No.: PCT/US2023/024167

§ 371 Date: Nov. 20, 2024

Related U.S. Application Data
(60) Provisional application No. 63/348,358, filed on Jun. 2, 2022.

(51) Int. Cl. C12N 9/22; A61K 31/7105; A61K 38/46; A61K 48/00; C12N 9/12; C12N 15/113

(52) U.S. Cl. CPC C12N 9/226; A61K 31/7105; A61K 38/465; A61K 48/0025; A61K 48/005; C12N 9/1252; C12N 15/113; C12Y 207/07007; C12N 2310/20; C12N 2310/531

(57) ABSTRACT

The present invention relates to the field of genomic engineering. In particular, a chimeric prime editing (cPE) system is disclosed comprising elements including, but not limited to a Cas9 nickase (nCas9)/high fidelity nucleotide polymerase (HFNTPol) RNA, one or more single guide RNAs (sgRNAs), and a chimeric prime editor template oligonucleotide (cpetODN) comprising a deoxyribonucleic acid nucleotide polymerase template (NPT) and a primer binding site. For example, the sgRNA and the cpetODN are ligated into a single oligonucleotide. Alternatively, the sgRNA and the cpetODN are free and independent molecules (e.g., modular). This cPE system results in precise and efficient genome editing in cells and in adult mouse liver which is advantageous over conventional sgRNA prime editor fusion constructs. This flexible and modular system is an improvement in the art to obtain precise genome editing.

### FIELD OF THE INVENTION

The present invention relates to the field of genomic engineering. In particular, a chimeric prime editing (cPE) system is disclosed comprising elements including, but not limited to a Cas9 nickase (nCas9), a high fidelity nucleotide polymerase (HFNTPol), a single guide RNA (sgRNA), and a chimeric prime editor template polynucleotide (cpetPN) comprising a deoxyribonucleic acid nucleotide polymerase template (NPT) and a primer binding site. The HFNTPol can be either a high fidelity RNA-dependent DNA polymerase (e.g., a high fidelity reverse transcriptase; HFRT) or a high fidelity DNA-dependent DNA polymerase (HFDNAPol). The HFNP can be fused or tethered to the nCas9, or separate and untethered (modular). This cPE system results in precise and efficient genome editing in cells and in adult mouse liver and is advantageous over conventional nucleotide polymerase and sgRNA prime editor fusion constructs. This flexible and modular system is an improvement in the art to obtain precise genome editing.

### BACKGROUND

Correction of genetic mutations in vivo has broad potential therapeutic application for a range of human genetic diseases. Prime editors (PE) composed of a Cas9 nickase fused to an engineered reverse transcriptase have enabled precise nucleotide changes, sequence insertions and deletions. Anzalone et al., “Search-and-replace genome editing without double-strand breaks or donor DNA” Nature 576:149-157 (2019).

This innovative technology does not induce double-stranded DNA breaks and does not require a donor DNA template in conjunction with homology-directed repair to introduce precise sequence changes into the genome. The ability to precisely install or correct pathogenic mutations makes prime editors an excellent tool to perform somatic genome editing.

Unlike base editing systems, prime editors can introduce any nucleotide substitution as well as insertions and deletions, and do not suffer from the challenges of bystander base conversion. These abilities may provide important advantages in some sequence contexts. Prime editor consists of a Cas9 nickase (H840A)-reverse transcriptase (RT) fusion protein paired with a pegRNA with desired edits. However, the potential of clinical use of PE is hampered by the large size for delivery (total length >6.3 kb). Furthermore, the lengths of the DNA insertions that can be installed efficiently by canonical PE systems described thus far is limited (<100 nt).

What is needed in the art is a PE system that is more stable, reliable, efficient, and accurate than conventional PE platforms, and that can generate genomic insertions and other edits with higher accuracy, greater length, or both.

### SUMMARY OF THE INVENTION

The present invention relates to the field of genomic engineering. In particular, a chimeric prime editing (cPE) system is disclosed comprising elements including, but not limited to a Cas9 nickase (nCas9), a high-fidelity nucleotide polymerase (HFNTPol), a single guide RNA (sgRNA), and a chimeric prime editor template polynucleotide (cpetPN) comprising a deoxyribonucleic acid nucleotide polymerase template (NPT) and a primer binding site (PBS). The HFNTPol can be either a high-fidelity RNA-dependent DNA polymerase (e.g., a high fidelity reverse transcriptase; HFRT) or a high-fidelity DNA-dependent DNA polymerase (HFDNAPol). The HFNTPol can be fused or tethered to the nCas9, or separate and untethered (modular). This cPE system results in precise and efficient genome editing in cells and in adult mouse liver which is advantageous over conventional sgRNA prime editor fusion constructs. This flexible and modular system is an improvement in the art to obtain precise genome editing.

In one embodiment, the present invention contemplates a high fidelity chimeric prime editing system comprising: i) a Cas9 nickase; ii) a high fidelity Phi29 nucleotide polymerase protein (HFPhi29NTPol); iii) a single guide RNA, and iv) a chimeric prime editor template oligonucleotide comprising a ribonucleic acid region that at least partially encodes a primer binding site (PBS) and a deoxyribonucleic acid region that at least partially encodes a nucleotide polymerase template (NPT). In one embodiment, the deoxyribonucleic acid region further at least partially encodes the PBS. In one embodiment, the ribonucleic acid region further at least partially encodes the NPT. In one embodiment, the ribonucleic acid region comprises a 3′-terminus with at least one chemical modification. In one embodiment, the deoxyribonucleic acid region comprises a 5′-terminus appended to a ribonucleic acid stem loop. In one embodiment, the ribonucleic acid stem loop is an MS2 stem loop. In one embodiment, the HFPhi29NTPol is a high fidelity deoxyribonucleic acid polymerase. In one embodiment, the HFPhi29NTPol is a high fidelity reverse transcriptase. In one embodiment, the HFPhi29NTPol comprises an error rate of approximately 1×10⁻⁶-10⁻⁷ nucleotides. In one embodiment, the HFPhi29NTPol comprises a processivity that reads an NPT of greater than 100 nucleotides (nts). In one embodiment, the HFPhi29NTPol comprises a deoxyribonucleotide triphosphate affinity (dNTP) of approximately 1-100 nanomolar. In one embodiment, the Cas9 nickase and the Phi29 nucleotide polymerase protein are ligated, attached or tethered. In one embodiment, the system is modular wherein the Cas9 nickase and the Phi29 nucleotide polymerase protein are not ligated, attached or tethered. In one embodiment, the single guide RNA is a prime editor guide RNA (pegRNA). In one embodiment, the at least one chemical modification is selected from the group consisting of 2′-O-methyl (2′-Ome), phosphorothioate (PS), locked nucleic acids (LNA), phosphatidylserine and fluoride (F). In one embodiment, the chimeric prime editor template oligonucleotide is circularized. In one embodiment, the chimeric prime editor template oligonucleotide is linear.

In one embodiment, the present invention contemplates a high fidelity chimeric prime editing system comprising: i) a first ribonucleic acid (RNA) encoding a Cas9 nickase protein and a high fidelity Phi29 nucleotide polymerase protein (HFPhi29NTPol); and ii) a second RNA encoding a single guide RNA and a chimeric prime editor template oligonucleotide comprising a ribonucleic acid region that at least partially encodes a primer binding site (PBS) and a deoxyribonucleic acid region that at least partially encodes a nucleotide polymerase template (NPT). In one embodiment, the deoxyribonucleic acid region further at least partially encodes the PBS. In one embodiment, the ribonucleic acid region further at least partially encodes the NPT. In one embodiment, the deoxyribonucleic acid region comprises a 5′-terminus appended to a ribonucleic acid stem loop. In one embodiment, the ribonucleic acid stem loop is an MS2 stem loop. In one embodiment, the ribonucleic acid region comprises a 3′-terminus with at least one chemical modification. In one embodiment, the deoxyribonucleic acid region comprises a 5′-terminus appended to a ribonucleic acid stem loop. In one embodiment, the HFPhi29NTPol is a high fidelity deoxyribonucleotide polymerase. In one embodiment, the HFPhi29NTPol is a high fidelity reverse transcriptase. In one embodiment, the HFPhi29NTPol comprises an error rate of approximately 1×10⁻⁶-10⁻⁷ nucleotides. In one embodiment, the HFPhi29NTPol comprises a processivity that reads an NPT of greater than 100 nucleotides (nts). In one embodiment, the HFPhi29NTPol comprises a deoxynucleotide triphosphate (dNTP) affinity of approximately 1-100 nanomolar. In one embodiment, the single guide RNA is a prime editor guide RNA (pegRNA). In one embodiment, the at least one chemical modification is selected from the group consisting of 2′-O-methyl (2′-Ome), phosphorothioate (PS), locked nucleic acids (LNA), phosphatidylserine and fluoride (F). In one embodiment, the chimeric prime editor template oligonucleotide is circularized. In one embodiment, the chimeric prime editor template oligonucleotide is linear. In one embodiment, a combination of the first and the second RNA are less then 4.5 kB.

In one embodiment, the present invention contemplates a composition, comprising: a) a ribonucleic acid (RNA) delivery system; and b) a high fidelity chimeric prime editing system comprising: i) a first ribonucleic acid (RNA) encoding a Cas9 nickase protein and a high fidelity Phi29 nucleotide polymerase protein (HFPhi29NTPol); and ii) a second RNA encoding a single guide RNA and a chimeric prime editor template oligonucleotide comprising a ribonucleic acid region that at least partially encodes a primer binding site (PBS) and a deoxyribonucleic acid region that at least partially encodes a nucleotide polymerase template (NPT). In one embodiment, the deoxyribonucleic acid region further at least partially encodes the PBS. In one embodiment, the ribonucleic acid region further at least partially encodes the NPT. In one embodiment, the deoxyribonucleic acid region comprises a 5′-terminus appended to a ribonucleic acid stem loop. In one embodiment, the ribonucleic acid stem loop is an MS2 stem loop. In one embodiment, the ribonucleic acid region comprises a 3′-terminus with at least one chemical modification. In one embodiment, the HFPhi29NTPol is a high fidelity deoxyribonucleotide polymerase. In one embodiment, the HFPhi29NTPol is a high fidelity reverse transcriptase. In one embodiment, the HFPhi29NTPol comprises an error rate of approximately 1×10⁻⁶-10⁻⁷ nucleotides. In one embodiment, the HFPhi29NTPol comprises a processivity that reads an NPT of greater than 100 nucleotides (nts). In one embodiment, the HFPhi29NTPol comprises a deoxynucleotide triphosphate (dNTP) affinity of approximately 1-100 nanomolar. In one embodiment, the NPT is a deoxyribonucleic acid nucleotide polymerase template (dNPT). In one embodiment, the single guide RNA is a prime editor guide RNA (pegRNA). In one embodiment, the at least one chemical modification is selected from the group consisting of 2′-O-methyl (2′-Ome), phosphorothioate (PS), locked nucleic acids (LNA), phosphatidylserine and fluoride (F). In one embodiment, the chimeric prime editor template oligonucleotide is circularized. In one embodiment, the chimeric prime editor template oligonucleotide is linear. In one embodiment, the combination of the first and the second RNA are less than 4.5 kB. In one embodiment, the RNA delivery system is an adeno-associated virus (AAV) delivery system. In one embodiment, the RNA delivery system is an mRNA delivery system. In one embodiment, the RNA delivery system is a ribonucleoprotein delivery system. In one embodiment, the RNA delivery system is a microparticle system. In one embodiment, the RNA delivery system is a liposome system.

In one embodiment, the present invention contemplates a method, comprising; a) providing; i) a patient expressing at least one symptom of a genetic disease or disorder, and ii) a pharmaceutically acceptable composition comprising; i) an RNA delivery system; and ii) a high fidelity chimeric prime editing system comprising a first ribonucleic acid (RNA) encoding a Cas9 nickase protein and a high fidelity Phi29 nucleotide polymerase protein (HFPhi29NTPol), and a second RNA encoding a single guide RNA and a chimeric prime editor template oligonucleotide comprising a ribonucleic acid region that at least partially encodes a primer binding site (PBS) and a deoxyribonucleic acid region that at least partially encodes a nucleotide polymerase template (NPT); and b) administering the pharmaceutically acceptable composition to said patient, wherein said at least one symptom of a genetic disease or disorder is reduced. In one embodiment, the deoxyribonucleic acid region further at least partially encodes the PBS. In one embodiment, the ribonucleic acid region further at least partially encodes the NPT. In one embodiment, the deoxyribonucleic acid region comprises a 5′-terminus appended to a ribonucleic acid stem loop. In one embodiment, the ribonucleic acid stem loop is an MS2 stem loop. In one embodiment, the ribonucleic acid region comprises a 3′-terminus with at least one chemical modification. In one embodiment, the HFPhi29NTPol is a high fidelity deoxyribonucleotide polymerase. In one embodiment, the HFPhi29NTPol is a high fidelity reverse transcriptase. In one embodiment, the HFPhi29NTPol comprises an error rate of approximately 1×10⁻⁶-10⁻⁷ nucleotides. In one embodiment, the HFPhi29NTPol comprises a processivity that read an NPT of greater than 100 nucleotides (nts). In one embodiment, the HFPhi29NTPol comprises a deoxynucleotide triphosphate (dNTP) affinity of approximately 1-100 nanomolar. In one embodiment, the administering further comprises expressing the first RNA and the second RNA. In one embodiment, the administering further comprises translating said encoded Cas9 nickase protein and HFPhi29NTPol. In one embodiment, the at least one chemical modification is selected from the group consisting of 2′-O-methyl (2′-Ome), phosphorothioate (PS), locked nucleic acids (LNA), phosphatidylserine and fluoride (F). In one embodiment, the chimeric prime editor template oligonucleotide is circularized. In one embodiment, the chimeric prime editor oligo nucleotide is linear. In one embodiment, a combination of the first and the second RNA are less than 4.5 kB. In one embodiment, the RNA delivery system comprises an adeno-associated virus system. In one embodiment, the RNA delivery system is a ribonucleoprotein delivery system or an mRNA delivery system. In one embodiment, the RNA delivery system is a microparticle system. In one embodiment, the RNA delivery system is a liposome system. In one embodiment, the single guide RNA hybridizes to a gene including, but not limited to, a HBB gene, a HEXA gene, a PSEN1 gene, a PRNP gene, an IDS gene and an IDUA gene.

In one embodiment, the present invention contemplates a chimeric prime editing system comprising: i) a Cas9 nickase; ii) a nucleotide polymerase protein comprising at least one high fidelity characteristic; iii) a single guide RNA, and iv) a chimeric prime editor template oligonucleotide comprising a ribonucleic acid region that at least partially encodes a primer binding site (PBS) and a deoxyribonucleic acid region that at least partially encodes a nucleotide polymerase template (NPT). In one embodiment, the deoxyribonucleic acid region further at least partially encodes the PBS. In one embodiment, the ribonucleic acid region further at least partially encodes the NPT. In one embodiment, the deoxyribonucleic acid region comprises a 5′-terminus appended to a ribonucleic acid stem loop. In one embodiment, the ribonucleic acid stem loop is an MS2 stem loop. In one embodiment, the ribonucleic acid region comprises a 3′-terminus with at least one chemical modification. In one embodiment, the high fidelity characteristic is a an error rate of approximately 1×10⁻⁶-10⁻⁷ nucleotides. In one embodiment, the high fidelity characteristic is a processivity that reads an NPT of greater than 100 nucleotides (nts). In one embodiment, the high fidelity characteristic comprises a deoxynucleotide triphosphate (dNTP) affinity of approximately 1-100 nanomolar. In one embodiment, the nucleotide polymerase protein is a deoxyribonucleic acid polymerase. In one embodiment, the nucleotide polymerase protein is a reverse transcriptase. In one embodiment, the Cas9 nickase and the nucleotide polymerase protein are ligated, attached or tethered. In one embodiment, the system is modular, wherein the Cas9 nickase and the high fidelity nucleotide polymerase protein are not ligated, attached or tethered. In one embodiment, the single guide RNA is a prime editor guide RNA (pegRNA). In one embodiment, the at least one chemical modification is selected from the group consisting of 2′-O-methyl (2′-Ome), phosphorothioate (PS), locked nucleic acids (LNA), phosphatidylserine and fluoride (F). In one embodiment, the chimeric prime editor template oligonucleotide is circularized. In one embodiment, the chimeric prime editor template oligonucleotide is linear.

In one embodiment, the present invention contemplates a chimeric prime editing system comprising: i) a first ribonucleic acid (RNA) encoding a Cas9 nickase protein and a nucleotide polymerase protein comprising at least one high fidelity characteristic; and ii) a second RNA encoding a single guide RNA and a chimeric prime editor template oligonucleotide comprising a ribonucleic acid region that at least partially encodes a primer binding site (PBS) and a deoxyribonucleic acid region that at least partially encodes a nucleotide polymerase template (NPT). In one embodiment, the deoxyribonucleic acid region further at least partially encodes the PBS. In one embodiment, the ribonucleic acid region further at least partially encodes the NPT. In one embodiment, the deoxyribonucleic acid region comprises a 5′-terminus appended to a ribonucleic acid stem loop. In one embodiment, the ribonucleic acid stem loop is an MS2 stem loop. In one embodiment, the ribonucleic acid region comprises a 3′-terminus with at least one chemical modification. In one embodiment, the high fidelity characteristic is a an error rate of approximately 1×10⁻⁶¹-10⁻⁷ nucleotides. In one embodiment, the high fidelity characteristic is a processivity that reads an NPT of greater than 100 nucleotides (nts). In one embodiment, the high fidelity characteristic comprises a deoxyribonucleotide triphosphate (dNTP) affinity of approximately 1-100 nanomolar. In one embodiment, the nucleotide polymerase protein is a deoxyribonucleotide polymerase. In one embodiment, the nucleotide polymerase protein is a reverse transcriptase. In one embodiment, the single guide RNA is a prime editor guide RNA (pegRNA). In one embodiment, the at least one chemical modification is selected from the group consisting of 2′-O-methyl (2′-Ome), phosphorothioate (PS), locked nucleic acids (LNA), phosphatidylserine and fluoride (F). In one embodiment, the chimeric prime editor template oligonucleotide is circularized. In one embodiment, the chimeric prime editor oligonucleotide is linear. In one embodiment, a combination of the first and the second RNA are less then 4.5 kB.

In one embodiment, the present invention contemplates a composition, comprising: a) a ribonucleic acid (RNA) delivery system; and b) a chimeric prime editing system comprising: i) a first ribonucleic acid (RNA) encoding a Cas9 nickase protein and a nucleotide polymerase protein comprising at least one high fidelity characteristic; and ii) a second RNA encoding a single guide RNA and a chimeric prime editor template oligonucleotide comprising a ribonucleic acid region that at least partially encodes a primer binding site (PBS) and a deoxyribonucleic acid region that at least partially encodes a nucleotide polymerase template (NPT). In one embodiment, the deoxyribonucleic acid region further at least partially encodes the PBS. In one embodiment, the ribonucleic acid region further at least partially encodes the NPT. In one embodiment, the deoxyribonucleic acid region comprises a 5′-terminus appended to a ribonucleic acid stem loop. In one embodiment, the ribonucleic acid stem loop is an MS2 stem loop. In one embodiment, the ribonucleic acid region comprises a 3′-terminus with at least one chemical modification. In one embodiment, the high fidelity characteristic is a an error rate of approximately 1×10⁻⁶-10⁻⁷ nucleotides. In one embodiment, the high fidelity characteristic is a processivity that reads an NPT of greater than 100 nucleotides (nts). In one embodiment, the high fidelity characteristic comprises a deoxyribonucleotide triphosphate affinity of approximately 1-100 nanomolar. In one embodiment, the nucleotide polymerase protein is a deoxyribonucleotide polymerase. In one embodiment, the nucleotide polymerase protein is a reverse transcriptase. In one embodiment, the single guide RNA is a prime editor guide RNA (pegRNA). In one embodiment, the at least one chemical modification is selected from the group consisting 2′-O-methyl (2′-Ome), phosphorothioate (PS), locked nucleic acids (LNA), phosphatidylserine and fluoride (F). In one embodiment, the chimeric prime editor template oligonucleotide is circularized. In one embodiment, the chimeric prime editor template oligonucleotide is linear. In one embodiment, a combination of the first and the second RNA are less than 4.5 kB. In one embodiment, the RNA delivery system is an adeno-associated virus (AAV) delivery system. In one embodiment, the RNA delivery system is an mRNA delivery system. In one embodiment, the RNA delivery system is a ribonucleoprotein delivery system. In one embodiment, the RNA delivery system is a microparticle system. In one embodiment, the RNA delivery system is a liposome system.

In one embodiment, the present invention contemplates a method, comprising; a) providing; i) a patient expressing at least one symptom of a genetic disease or disorder, and ii) a pharmaceutically acceptable composition comprising; i) an RNA delivery system; and ii) a chimeric prime editing system comprising a first ribonucleic acid (RNA) encoding a Cas9 nickase protein and a nucleotide polymerase protein comprising at least one high fidelity characteristic, and a second RNA encoding a single guide RNA and a chimeric prime editor template oligonucleotide comprising a ribonucleic acid region that at least partially encodes a primer binding site (PBS) and a deoxyribonucleic acid region that at least partially encodes a nucleotide polymerase template (NPT); and b) administering the pharmaceutically acceptable composition to said patient, wherein said at least one symptom of a genetic disease or disorder is reduced. In one embodiment, the deoxyribonucleic acid region further at least partially encodes the PBS. In one embodiment, the ribonucleic acid region further at least partially encodes the NPT. In one embodiment, the deoxyribonucleic acid region comprises a 5′-terminus appended to a ribonucleic acid stem loop. In one embodiment, the ribonucleic acid stem loop is an MS2 stem loop. In one embodiment, the ribonucleic acid region comprises a 3′-terminus with at least one chemical modification. In one embodiment, the high fidelity characteristic is an error rate of approximately 1×10⁻⁶-10⁻⁷ nucleotides. In one embodiment, the high fidelity characteristic is a processivity that reads an NPT of greater than 100 nucleotides (nts). In one embodiment, the high fidelity characteristic comprise a nucleotide triphosphate (dNTP) affinity of approximately 1-100 nanomolar. In one embodiment, the nucleotide polymerase protein is a deoxyribonucleotide polymerase. In one embodiment, the nucleotide polymerase protein is a reverse transcriptase. In one embodiment, the administering further comprises expressing the first RNA and the second RNA. In one embodiment, the administering further comprises translating said encoded Cas9 nickase protein and nucleotide polymerase protein. In one embodiment, the at least one chemical modification is selected from the group consisting of 2′-O-methyl (2′-Ome), phosphorothioate (PS), locked nucleic acids (LNA), phosphatidylserine and fluoride (F). In one embodiment, the chimeric prime editor template oligonucleotide is circularized. In one embodiment, the chimeric prime editor template oligonucleotide is linear. In one embodiment, a combination of the first and the second RNA are less than 4.5 kB. In one embodiment, the RNA delivery system comprises an adeno-associated virus system. In one embodiment, the RNA delivery system is a ribonucleoprotein delivery system or an mRNA delivery system. In one embodiment, the RNA delivery system is a microparticle system. In one embodiment, the RNA delivery system is a liposome system. In one embodiment, the single guide RNA hybridizes to a gene including, but not limited to, a HBB gene, a HEXA gene, a PSEN1 gene, a PRNP gene, an IDS gene and an IDUA gene.

In one embodiment, the present invention contemplates a chimeric prime editor template oligonucleotide comprising a deoxyribonucleic acid (DNA) region at least partially encoding a nucleotide polymerase template (NPT) ligated to a ribonucleic acid (RNA) region at least partially encoding a primer binding site (PBS). In one embodiment, the deoxyribonucleic acid region further at least partially encodes the PBS. In one embodiment, the ribonucleic acid region further at least partially encodes the NPT. In one embodiment, the deoxyribonucleic acid region comprises a 5′-terminus appended to a ribonucleic acid stem loop. In one embodiment, the ribonucleic acid stem loop is an MS2 stem loop. In one embodiment, the ribonucleic acid region comprises a 3′-terminus with at least one chemical modification. In one embodiment, a 3′-terminus of the DNA region is ligated to a 5′-terminus of the RNA acid region. In one embodiment, the DNA region completely encodes the nucleotide polymerase protein. In one embodiment, the RNA region completely encodes the primer binding site. In one embodiment, the at least one chemical modification is selected from the group consisting of 2′-O-methyl (2′-Ome), phosphorothioate (PS), locked nucleic acids (LNA), phosphatidylserine and fluoride (F). In one embodiment, the nucleotide polymerase includes, but is not limited to, a DNA polymerase and a reverse transcriptase. In one embodiment, the DNA polymerase is a high fidelity DNA polymerase. In one embodiment, the high fidelity DNA polymerase is a Phi29 DNA polymerase. In one embodiment, the high fidelity DNA polymerase is a T4 DNA polymerase, T7 DNA polymerase and a truncated E. coli DNA polymerase I (e.g., Klenow fragment). In one embodiment, the reverse transcriptase includes, but is not limited to, an M_MLV reverse transcriptase and an mSA reverse transcriptase. In one embodiment, the primer binding site is complementary to a gene including, but not limited to, a HBB gene, a HEXA gene, a PSEN1 gene, a PRNP gene, an IDS gene and an IDUA gene. In one embodiment, the chimeric prime editor template oligonucleotide further comprises a biotin molecule.

In one embodiment, the present invention contemplates a chimeric prime editing system comprising: i) a Cas9 nickase; ii) a high fidelity nucleotide polymerase protein (HFNTPol); iii) a single guide RNA (sgRNA), and iv) a ribonucleic acid comprising a primer binding site (PBS) ligated to a nucleotide polymerase template (NPT). In one embodiment, the high fidelity nucleotide polymerase protein is a high fidelity deoxyribonucleic acid polymerase protein (HFDNAPol). In one embodiment, the high fidelity nucleotide polymerase protein is a high fidelity reverse transcriptase (HFRT). In one embodiment, the NPT comprises a deoxyribonucleic acid template (dNPT). In one embodiment, the NPT comprises a ribonucleic acid template (rNPT). In one embodiment, the Cas9 nickase and the high fidelity nucleotide polymerase protein are ligated, attached or tethered. In one embodiment, the system is modular, wherein the Cas9 nickase and the high fidelity nucleotide polymerase protein are not ligated, attached or tethered. In one embodiment, the single guide RNA is a prime editor guide RNA (pegRNA). In one embodiment, the ribonucleic acid is a chimeric prime editor template oligonucleotide (cpetODN). In one embodiment, the cpetODN comprises deoxyribonucleic acid nucleotides. In one embodiment, the cpetODN comprises deoxyribonucleic acid nucleotides and ribonucleic acid nucleotides. In one embodiment, the cpetODN comprises at least one chemical modification. In one embodiment, the at least one chemical modification is selected from the group consisting of 2′-O-methyl (2′-Ome), phosphorothioate (PS), locked nucleic acids (LNA), phosphatidylserine and fluoride (F). In one embodiment, the primer binding site is a ribonucleic acid primer binding site. In one embodiment, the primer binding site is a chimeric primer binding site comprising deoxyribonucleic acids and ribonucleic acids. In one embodiment, the primer binding site is a deoxyribonucleic acid chimeric primer binding site. In one embodiment, the cpetODN is circularized. In one embodiment, the cpetODN is linear.

In one embodiment, the present invention contemplates a chimeric prime editing system comprising: i) a first ribonucleic acid (RNA) encoding a Cas9 nickase protein and a high fidelity nucleotide polymerase protein (HFNTPol); and ii) a second RNA encoding a sequence selected from the group including, but not limited to, a single guide RNA (sgRNA), a primer binding site (PBS) and a nucleotide polymerase template (NPT). In one embodiment, the high fidelity nucleotide polymerase protein is a high fidelity deoxyribonucleotide polymerase protein (HFDNAPol). In one embodiment, the high fidelity nucleotide polymerase protein is a high fidelity reverse transcriptase (HFRT). In one embodiment, the NPT is a deoxyribonucleic acid template (dNPT). In one embodiment, the NPT is a ribonucleic acid template (rNPT). In one embodiment, the single guide RNA is a prime editor guide RNA (pegRNA). In one embodiment, the second RNA comprises a chimeric prime editor template oligonucleotide (cpetODN). In one embodiment, the cpetODN comprises deoxyribonucleic acid nucleotides. In one embodiment, the cpetODN comprises deoxyribonucleic acid nucleotides and ribonucleic acid nucleotides. In one embodiment, the cpetODN comprises at least one chemical modification. In one embodiment, the at least one chemical modification is selected from the group consisting of 2′-O-methyl (2′-Ome), phosphorothioate (PS), locked nucleic acids (LNA), phosphatidylserine and fluoride (F). In one embodiment, the primer binding site is a ribonucleic acid primer binding site. In one embodiment, the primer binding site is a chimeric primer binding site comprising deoxyribonucleic acids and ribonucleic acids. In one embodiment, the primer binding site is a deoxyribonucleic acid chimeric primer binding site. In one embodiment, the cpetODN is circularized. In one embodiment, the cpetODN is linear. In one embodiment, the first RNA is less than 4.5 kB. In one embodiment, the second RNA is less than 4.5 kB. In one embodiment, the combination of the first and the second RNA is less then 4.5 kB.

In one embodiment, the present invention contemplates a composition, comprising: a) a ribonucleic acid (RNA) delivery system; and b) a high fidelity chimeric prime editing system comprising: i) a first ribonucleic acid (RNA) encoding a Cas9 nickase protein and a high fidelity nucleotide polymerase protein (HFNTPol); and ii) a second RNA encoding a sequence including, but not limited to a single guide RNA (sgRNA), a primer binding site (PBS) and a deoxyribonucleic acid nucleotide polymerase template (NPT). In one embodiment, the high fidelity nucleotide polymerase protein is a high fidelity deoxyribonucleotide polymerase protein (HFDNAPol). In one embodiment, the high fidelity nucleotide polymerase protein is a high fidelity reverse transcriptase (HFRT). In one embodiment, the NPT is a deoxyribonucleic acid template (dNPT). In one embodiment, the NPT is a ribonucleic acid template (rNPT). In one embodiment, the single guide RNA is a prime editor guide RNA (pegRNA). In one embodiment, the second RNA comprises a chimeric prime editor template oligonucleotide (cpetODN). In one embodiment, the cpetODN comprises deoxyribonucleic acid nucleotides. In one embodiment, the cpetODN comprises deoxyribonucleic acid nucleotides and ribonucleic acid nucleotides. In one embodiment, the cpetODN comprises at least one chemical modification. In one embodiment, the at least one chemical modification is selected from the group consisting 2′-O-methyl (2′-Ome), phosphorothioate (PS), locked nucleic acids (LNA), phosphatidylserine and fluoride (F). In one embodiment, the primer binding site is a ribonucleic acid primer binding site. In one embodiment, the primer binding site is a chimeric primer binding site comprising deoxyribonucleic acids and ribonucleic acids. In one embodiment, the primer binding site is a deoxyribonucleic acid chimeric primer binding site. In one embodiment, the cpetODN is circularized. In one embodiment, the cpetODN is linear. In one embodiment, the first RNA is less than 4.5 kB. In one embodiment, the second RNA is less than 4.5 kB. In one embodiment, the combination of the first and the second RNA is less than 4.5 kB. In one embodiment, the RNA delivery system is an adeno-associated virus (AAV) delivery system. In one embodiment, the RNA delivery system is an mRNA delivery system. In one embodiment, the RNA delivery system is a ribonucleoprotein delivery system. In one embodiment, the RNA delivery system is a microparticle system. In one embodiment, the RNA delivery system is a liposome system.

In one embodiment, the present invention contemplates a method, comprising; a) providing; i) a patient expressing at least one symptom of a genetic disease or disorder, and ii) a pharmaceutically acceptable composition comprising; i) an RNA delivery system; and ii) a high fidelity chimeric prime editing system comprising a first ribonucleic acid (RNA) encoding a Cas9 nickase protein and a high fidelity nucleotide polymerase protein (HFNTPol), and a second RNA encoding a sequence including, but not limited to, a single guide RNA (sgRNA), a primer binding site (PBS) and a nucleotide polymerase template (NPT); b) administering the pharmaceutically acceptable composition to said patient, wherein said at least one symptom of a genetic disease or disorder is reduced. In one embodiment, the high fidelity nucleotide polymerase protein is a high fidelity deoxyribonucleotide polymerase protein (HFDNAPol). In one embodiment, the high fidelity nucleotide polymerase protein is a high fidelity reverse transcriptase (HFRT). In one embodiment, the NPT is a deoxyribonucleic acid template (dNPT). In one embodiment, the NPT is a ribonucleic acid template (rNPT). In one embodiment, the administering further comprises expressing the first RNA and the second RNA. In one embodiment, the administering further comprises translating said encoded Cas9 nickase protein. In one embodiment, the administering further comprises translating said encoded nucleotide polymerase protein. In one embodiment, the second RNA comprises a chimeric prime editor template oligonucleotide (cpetODN). In one embodiment, the cpetODN comprises deoxyribonucleic acid nucleotides. In one embodiment, the cpetODN comprises deoxyribonucleic acid nucleotides and ribonucleic acid nucleotides. In one embodiment, the cpetODN comprises at least one chemical modification. In one embodiment, the at least one chemical modification is selected from the group consisting of 2′-O-methyl (2′-Ome), phosphorothioate (PS), locked nucleic acids (LNA), phosphatidylserine and fluoride (F). In one embodiment, the primer binding site is a ribonucleic acid primer binding site. In one embodiment, the primer binding site is a chimeric primer binding site comprising deoxyribonucleic acids and ribonucleic acids. In one embodiment, the primer binding site is a deoxyribonucleic acid chimeric primer binding site. In one embodiment, the cpetODN is circularized. In one embodiment, the cpetODN is linear. In one embodiment, the first RNA is less than 4.5 kB. In one embodiment, the second RNA is less than 4.5 kB. In one embodiment, the combination of the first and the second RNA is less than 4.5 kB. In one embodiment, the RNA delivery system comprises an adeno-associated virus system. In one embodiment, the RNA delivery system is a ribonucleoprotein delivery system or an mRNA delivery system. In one embodiment, the RNA delivery system is a microparticle system. In one embodiment, the RNA delivery system is a liposome system. In one embodiment, the single guide RNA hybridizes to a gene including, but not limited to, a HBB gene, a HEXA gene, a PSEN1 gene, a PRNP gene, an IDS gene and an IDUA gene.

### Definitions

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but also plural entities and also includes the general class of which a specific example may be used for illustration. The terminology herein is used to describe specific embodiments of the invention, but their usage does not delimit the invention, except as outlined in the claims.

The term “about” or “approximately” as used herein, in the context of any of any assay measurements refers to +/−5% of a given measurement.

The term “nucleotide polymerase” as used herein, refers to an enzyme which catalyzes the linking of the 3′-hydroxyl group of a terminal nucleotide to the 5′-phosphate of a free nucleoside. If the polymerase transcribes a ribonucleic acid polymer it is referred to as a reverse transcriptase (e.g., an RNA-dependent DNA polymerase). If the polymerase transcribes a deoxyribonucleic acid polymer it is referred to as a DNA-dependent DNA polymerase. Some nucleotide polymerases are either an RNA or DNA polymerase, while other nucleotide polymerases can function as both an RNA and DNA polymerase.

The term “high fidelity” as used herein, refers to a nucleotide polymerase classification based upon high performance by measure of accuracy and efficiency. For example, one characteristic of a high fidelity nucleotide polymerase is an error rate of approximately 1×10⁻⁶-10⁻⁷ nucleotides or less. As contemplated herein, high fidelity polymerases having an error rate of approximately 1×10⁻⁶-10⁻⁷ nucleotides or less include, but are not limited to, the Phi29 polymerase, the T7 polymerase, the T7 polymerase and the DNA Pol I Klenow fragment polymerase. Another characteristic of a high fidelity nucleotide polymerase is an ability to read a template oligonucleotide that is greater than 100 nucleotides (nts). As contemplated herein, high fidelity polymerases having a read length of >100 nts or greater include, but are not limited to, the Phi29 polymerase, the T7 polymerase, the T7 polymerase and the DNA Pot I Klenow fragment polymerase. Another characteristic of a high fidelity polymerase is an affinity for deoxynucleotide triphosphate (dNTP) of approximately 1-100 nanomolar. As contemplated herein, high fidelity polymerases have a dNTP affinity of approximately 1-100 nanomolar include, but are not limited to, the Phi29 polymerase, the T7 polymerase, the T7 polymerase and the DNA Pot I Klenow fragment polymerase.

The fidelity of a polymerase is the result of accurate replication of a desired template. Specifically, this involves multiple steps, including the ability to read a template strand, select the appropriate nucleoside triphosphate and insert the correct nucleotide at the 3′ primer terminus, such that Watson-Crick base pairing is maintained. In addition to effective discrimination of correct versus incorrect nucleotide incorporation, some polymerases possess a 3′→5′ exonuclease activity. This activity, known as “proofreading”, is used to excise incorrectly incorporated mononucleotides that are then replaced with the correct nucleotide. High-fidelity polymerases couple low misincorporation rates with proofreading activity to give a highly accurate replication of the target DNA of interest.

The term “processivity” as used herein, refers the total number of nucleotides being processed (e.g., polymerized, replicated, amplified etc.) in a single cycle of enzyme-substrate binding. Processivity often reflects synthesis rate and speed, as well as polymerase affinity for the template oligonucleotide or polynucleotide.

The term “read length” as used herein, refers to a quantitative measure of polymerase processivity as determined by the number of inserted nucleotides (nts).

The term “DNA polymerase editing” or “DPE” as used herein, refers to editing based on DNA-dependent DNA polymerases and exogenous templates. DPE is distinguished from the EvolvR system which does not employ an exogenous template and RT-based prime editing systems.

The term “DNA polymerase editing template” or “DPET” as used herein, refers to an exogenous, user-defined DNA polymerase template.

The term “catalytically impaired Cas9 nickase” or “nCas9”, as used herein refers to a mutated Cas9 which renders the nuclease able to cleave only one strand of deoxyribonucleic acid backbone. Depending on the position of the mutation within the Cas9 protein sequence either the target or non-target strand is cleaved. In the case of a prime editor the non-target strand is selectively cleaved.

The term “engineered reverse transcriptase” as used herein, refers to a protein that converts RNA into DNA and contains specific mutations that effect its activity efficiency. One example, of a reverse transcriptase is a Moloney murine leukemia virus reverse transcriptase (M-MLV RT).

The term “reverse transcriptase template” or “RTT” as used herein refers to a ribonucleic acid sequence that is utilized as a nucleic acid for a reverse transcriptase protein that is part of the prime editor complex as contemplated herein. Such templates provide the necessary information to edit a DNA sequence to support conversions including, but not limited to, base conversions, sequence insertions or sequence deletions.

The term “nucleotide polymerase template” or “NPT” as used herein refers to a deoxyribonucleic or a ribonucleic acid sequence and modifications thereof, that is utilized as a nucleic acid for a nucleotide polymerase protein (e.g., reverse transcriptase or a DNA-dependent DNA polymerase) that is part of the chimeric prime editor complex as contemplated herein. Such templates provide the necessary information to edit a DNA sequence to support conversions including, but not limited to, base conversions, sequence insertions or sequence deletions.

The term “primer binding site” or “PBS” as used herein, refers to a specific nucleic acid sequence within a pegRNA or a petRNA or a cpetODN that is complementary to the 3′ end of the nicked DNA strand. This allows annealing of the free 3′ end of the genomic DNA for extension by the nucleotide polymerase based on the template sequence encoded in the pegRNA, petRNA or cpetODN.

The term, “prime editing guide RNA molecule” or “pegRNA molecule” as used herein, refers to a Cas9 guide RNA molecule that encodes the crRNA-tracrRNA fused to a primer binding site (PBS) and a reverse transcriptase template (RTT) or a nucleotide polymerase template (NPT) nucleic acid sequence. The primer binding site hybridizes to a desired genomic sequence released by the binding and cleavage of the Cas9 nickase. The 3′ end of the genomic sequence is extended by the reverse transcriptase or the nucleotide polymerase based on its cognate template sequence.

The term, “petRNA”, as used herein, refers to an RNA molecule that encodes a “prime editor template (pet), comprising a primer binding site (PBS) and a reverse transcriptase template (RTT) or a nucleotide polymerase template (NPT). The petRNA may also be appended with a stem loop, such as an MS2 stem loop. For example, the MS2 stem loop is appended at a petRNA terminus. The petRNA may also be linear or circularized. The “petRNA” may also be a chimeric petRNA molecule (e.g., cpetRNA) wherein at least one deoxyribonucleic acid is present. For example, a cpetRNA may comprise a polymerase DNA template and a primer binding site that is all RNA, all DNA or a combination of both RNA and DNA. The “petRNA” may also be linear (e.g., linpet or LPET) or circular.

The term, “chimeric” as used herein, refers to an oligonucleotide comprising both deoxyribonucleic acids and ribonucleic acids.

The term “editing” or “gene editing” as used herein, refers to a genetic manipulation of a DNA sequence. Such a manipulation includes, but is not limited to, a base conversion, a sequence insertion and/or a sequence deletion.

The term “prime editing” as used herein, is a genome editing technology by which the genome of living organisms may be modified. Prime editing manipulates the genetic information of a targeted DNA site to essentially “rewrite” the coded sequences.

The term “prime editor” or “PE” as used herein, is a fusion protein comprising a catalytically impaired Cas9 endonuclease that can nick DNA and is fused to an engineered reverse transcriptase enzyme and attached to a prime editing guide RNA (pegRNA). The pegRNA is capable of programming the nCas9 to recognize a target site with the encoded crRNA-tracrRNA (as does a conventional single guide RNA). The resulting nicked genomic DNA can be extended by a reverse transcriptase or a nucleotide polymerase based on the pegRNA template sequence to contain a new sequence. Once one strand is recoded, cellular DNA repair pathways can cause conversion of the local DNA sequence to match the new sequence. Such manipulation includes, but is not limited to, insertions, deletions, and base-to-base conversions without the need for double strand breaks (DSBs) or donor DNA templates. For example, such prime editing may be performed by a Cas9 CRISPR platform programmed with a pegRNA, such as a catalytically impaired Cas9 nickase platform with an appropriate reverse transcriptase.

The term “conversion” as used herein, refers to any manipulation of a nucleic acid sequence that converts a mutated sequence into a wild type sequence, or a wild type sequence into a mutated sequence. For example, a converted sequence includes, but is not limited to, a base pair conversion, a nucleic acid sequence insertion or a nucleic acid sequence deletion.

The term “editing-related indels” as used herein, refers to the generation of off-target and/or unintended nucleotide sequence insertions created by a prime editor.

The term “split-intein prime editor protein” refers to a prime editor protein that has been split into amino-terminal (PE2-N) and carboxy-terminal (PE2-C) segments, which are then fused into a full length PE by a trans-splicing intein. This configuration imparts flexibility to the prime editor thereby facilitating a packaging into an adeno-associated virus (AAV).

As used herein, the term “CRISPRs” or “Clustered Regularly Interspaced Short Palindromic Repeats” refers to an acronym for DNA loci that contain multiple, short, direct repetitions of base sequences. Each repetition contains a series of bases followed by 30 or so base pairs known as “spacer” sequence. The spacers are short segments of DNA from a virus and may serve as a ‘memory’ of past exposures to facilitate an adaptive defense against future invasions. Doudna et al. Genome editing. The new frontier of genome engineering with CRISPR-Cas9” Science 346(6213):1258096 (2014).

As used herein, the term “Cas” or “CRISPR-associated (cas)” refers to genes often associated with CRISPR repeat-spacer arrays.

As used herein, the term “Cas9” refers to a nuclease from type II CRISPR systems, an enzyme specialized for generating double-strand breaks in DNA, with two active cutting sites (the HNH and RuvC domains), one for each strand of the double helix. tracrRNA and CRISPR RNA (crRNA) may be combined into a “single-guide RNA” (sgRNA) molecule that, mixed with Cas9, could find and cleave DNA targets through Watson-Crick pairing between the guide sequence within the sgRNA and the target DNA sequence, Jinek et al. A programmable dual-RNA-guided DNA endonuclease in adaptive bacterial immunity” Science 337(6096):816-821 (2012).

As used herein, the term “catalytically active Cas9” refers to an unmodified Cas9 nuclease comprising full nuclease activity.

The term “nickase” as used herein, refers to a nuclease that cleaves only a single DNA strand, either due to its natural function or because it has been engineered to cleave only a single DNA strand. Cas9 nickase variants that have either the RuvC or the HNH domain mutated provide control over which DNA strand is cleaved and which remains intact. Jinek et al., “A programmable dual-RNA-guided DNA endonuclease in adaptive bacterial immunity” Science 337(6096):816-821 (2012) and Cong et al. Multiplex genome engineering using CRISPR/Cas systems” Science 339(6121):819-823 (2013).

The term, “trans-activating crRNA”, “tracrRNA” as used herein, refers to a small trans-encoded RNA. For example, CRISPR/Cas (clustered, regularly interspaced short palindromic repeats/CRISPR-associated proteins) constitutes an RNA-mediated defense system, which protects against viruses and plasmids. This defensive pathway has three steps. First a copy of the invading nucleic acid is integrated into the CRISPR locus. Next, CRISPR RNAs (crRNAs) are transcribed from this CRISPR locus. The crRNAs are then incorporated into construct complexes, where the crRNA guides the complex to the invading nucleic acid and the Cas proteins degrade this nucleic acid. There are several pathways of CRISPR activation, one of which requires a tracrRNA, which plays a role in the maturation of crRNA. TracrRNA is complementary to the repeat sequence of the pre-crRNA, forming an RNA duplex. This is cleaved by RNase III, an RNA-specific ribonuclease, to form a crRNA/tracrRNA hybrid. This hybrid acts as a guide for the endonuclease Cas9, which cleaves the invading nucleic acid.

The term “protospacer adjacent motif” (or PAM) as used herein, refers to a DNA sequence that may be required for a Cas9/sgRNA to form an R-loop to interrogate a specific DNA sequence through Watson-Crick pairing of its guide RNA with the genome. The PAM specificity may be a function of the DNA-binding specificity of the Cas9 protein (e.g., a “protospacer adjacent motif recognition domain” at the C-terminus of Cas9).

The terms “protospacer adjacent motif recognition domain”, “PAM Interacting Domain” or “PID” as used herein, refers to a Cas9 amino acid sequence that comprises a binding site to a DNA target PAM sequence.

The term “binding site” as used herein, refers to any molecular arrangement having a specific tertiary and/or quaternary structure that undergoes a physical attachment or close association with a binding component. For example, the molecular arrangement may comprise a sequence of amino acids. Alternatively, the molecular arrangement may comprise a sequence of nucleic acids. Furthermore, the molecular arrangement may comprise a lipid bilayer or other biological material.

As used herein, the term “sgRNA” refers to single guide RNA used in conjunction with CRISPR associated systems (Cas). sgRNAs are a fusion of crRNA and tracrRNA and contain nucleotides of sequence complementary to the desired target site. Jinek et al., “A programmable dual-RNA-guided DNA endonuclease in adaptive bacterial immunity” Science 337(6096):816-821 (2012) Watson-Crick pairing of the sgRNA with the target site permits R-loop formation, which in conjunction with a functional PAM permits DNA cleavage or in the case of nuclease-deficient Cas9 allows binds to the DNA at that locus.

As used herein, the term “orthogonal” refers to targets that are non-overlapping, uncorrelated, or independent. For example, if two orthogonal Cas9 isoforms were utilized, they would employ orthogonal sgRNAs that only program one of the Cas9 isoforms for DNA recognition and cleavage. Esvelt et al., “Orthogonal Cas9 proteins for RNA-guided gene regulation and editing” Nat Methods 10(11):1116-1121 (2013). For example, this would allow one Cas9 isoform (e.g. S. pyogenes Cas9 or SpyCas9) to function as a nuclease programmed by a sgRNA that may be specific to it, and another Cas9 isoform (e.g. N. meningitidis Cas9 or NmeCas9) to operate as a nuclease-dead Cas9 that provides DNA targeting to a binding site through its PAM specificity and orthogonal sgRNA. Other Cas9s include S. aureus Cas9 or SauCas9 and A. naeslundii Cas9 or AnaCas9.

The term “truncated” as used herein, when used in reference to either a polynucleotide sequence or an amino acid sequence means that at least a portion of the wild type sequence may be absent. In some cases, truncated guide sequences within the sgRNA or crRNA may improve the editing precision of Cas9. Fu, et al. “Improving CRISPR-Cas nuclease specificity using truncated guide RNAs” Nat Biotechnol. 2014 March; 32(3):279-284 (2014).

The term “base pairs” as used herein, refer to specific nucleobases (also termed nitrogenous bases), that are the building blocks of nucleotide sequences that form a primary structure of both DNA and RNA. Double-stranded DNA may be characterized by specific hydrogen bonding patterns. Base pairs may include, but are not limited to, guanine-cytosine and adenine-thymine base pairs.

The term “specific genomic target” as used herein, refers to any pre-determined nucleotide sequence capable of binding to a Cas9 protein contemplated herein. The target may include, but may be not limited to, a nucleotide sequence complementary to a programmable DNA binding domain or an orthogonal Cas9 protein programmed with its own guide RNA, a nucleotide sequence complementary to a single guide RNA, a protospacer adjacent motif recognition sequence, an on-target binding sequence and an off-target binding sequence.

As used herein, the term “edit”, “editing” or “edited” refers to a method of altering a nucleic acid sequence of a polynucleotide (e.g., for example, a wild type naturally occurring nucleic acid sequence or a mutated naturally occurring sequence) by selective deletion of a specific genomic target or the specific inclusion of new sequence through the use of an exogenously supplied DNA template. Such a specific genomic target includes, but may be not limited to, a chromosomal region, mitochondrial DNA, a gene, a promoter, an open reading frame or any nucleic acid sequence.

The term “effective amount” as used herein, refers to a particular amount of a pharmaceutical composition comprising a therapeutic agent that achieves a clinically beneficial result (i.e., for example, a reduction of symptoms). Toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. Compounds that exhibit large therapeutic indices are preferred. The data obtained from these cell culture assays and additional animal studies can be used in formulating a range of dosage for human use. The dosage of such compounds lies preferably within a range of circulating concentrations that include the ED₅₀ with little or no toxicity. The dosage varies within this range depending upon the dosage form employed, sensitivity of the patient, and the route of administration.

The term “symptom”, as used herein, refers to any subjective or objective evidence of disease or physical disturbance observed by the patient. For example, subjective evidence is usually based upon patient self-reporting and may include, but is not limited to, pain, headache, visual disturbances, nausea and/or vomiting. Alternatively, objective evidence is usually a result of medical testing including, but not limited to, body temperature, complete blood count, lipid panels, thyroid panels, blood pressure, heart rate, electrocardiogram, tissue and/or body imaging scans.

The term “associated with” or “linked to” as used herein, refers to an art-accepted causal relationship between a genetic mutation and a medical condition or disease. For example, it is art-accepted that a patient having an HTT gene comprising a tandem CAG repeat expansion mutation has, or is a risk for, Huntington's disease.

The term “disease” or “medical condition”, as used herein, refers to any impairment of the normal state of the living animal or plant body or one of its parts that interrupts or modifies the performance of the vital functions. Typically manifested by distinguishing signs and symptoms, it is usually a response to: i) environmental factors (as malnutrition, industrial hazards, or climate); ii) specific infective agents (as worms, bacteria, or viruses); iii) inherent defects of the organism (as genetic anomalies); and/or iv) combinations of these factors.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

The term “attached” as used herein, refers to any interaction between a medium (or carrier) and a drug. Attachment may be reversible or irreversible. Such attachment includes, but is not limited to, covalent bonding, ionic bonding, Van der Waals forces or friction, and the like.

The term “administered” or “administering”, as used herein, refers to any method of providing a composition to a patient such that the composition has its intended effect on the patient. An exemplary method of administering is by a direct mechanism such as, local tissue administration (i.e., for example, extravascular placement), oral ingestion, transdermal patch, topical, inhalation, suppository etc.

The term “patient” or “subject”, as used herein, is a human or animal and need not be hospitalized. For example, out-patients, persons in nursing homes are “patients.” A patient may comprise any age of a human or non-human animal and therefore includes both adult and juveniles (i.e., children). It is not intended that the term “patient” connote a need for medical treatment, therefore, a patient may voluntarily or involuntarily be part of experimentation whether clinical or in support of basic science studies.

The term “derived from” as used herein, refers to the source of a sample, a compound or a sequence. In one respect, a sample, a compound or a sequence may be derived from an organism or particular species. In another respect, a sample, a compound or sequence may be derived from a larger complex or sequence.

The term “protein” as used herein, refers to any of numerous naturally occurring extremely complex substances (as an enzyme or antibody) that consist of amino acid residues joined by peptide bonds, contain the elements carbon, hydrogen, nitrogen, oxygen, usually sulfur. In general, a protein comprises amino acids having an order of magnitude within the hundreds.

The term “peptide” as used herein, refers to any of various amides that are derived from two or more amino acids by combination of the amino group of one acid with the carboxyl group of another and are usually obtained by partial hydrolysis of proteins. In general, a peptide comprises amino acids having an order of magnitude with the tens.

The term “polypeptide” refers to any of various amides that are derived from two or more amino acids by combination of the amino group of one acid with the carboxyl group of another and are usually obtained by partial hydrolysis of proteins. In general, a peptide comprises amino acids having an order of magnitude with the tens or larger.

The term “pharmaceutically” or “pharmacologically acceptable”, as used herein, refer to molecular entities and compositions that do not produce adverse, allergic, or other untoward reactions when administered to an animal or a human.

The term, “pharmaceutically acceptable carrier”, as used herein, includes any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

“Nucleic acid sequence” and “nucleotide sequence” as used herein refer to an oligonucleotide or polynucleotide, and fragments or portions thereof, and to DNA or RNA of genomic or synthetic origin which may be single- or double-stranded, and represent the sense or antisense strand.

The term “an isolated nucleic acid”, as used herein, refers to any nucleic acid molecule that has been removed from its natural state (e.g., removed from a cell and is, in a preferred embodiment, free of other genomic nucleic acid).

The terms “amino acid sequence” and “polypeptide sequence” as used herein, are interchangeable and to refer to a sequence of amino acids.

The term “portion” when used in reference to a nucleotide sequence refers to fragments of that nucleotide sequence. The fragments may range in size from 5 nucleotide residues to the entire nucleotide sequence minus one nucleic acid residue. When used in reference to an amino acid sequence refers to fragments of that amino acid sequence. The fragment may range in size from 2 amino acid residues to the entire amino acid sequence minus one amino acid residue.

As used herein, the terms “complementary” or “complementarity” are used in reference to “polynucleotides” and “oligonucleotides” (which are interchangeable terms that refer to a sequence of nucleotides) related by the base-pairing rules. For example, the sequence “C-A-G-T,” is complementary to the sequence “G-T-C-A.” Complementarity can be “partial” or “total.” “Partial” complementarity is where one or more nucleic acid bases is not matched according to the base pairing rules. “Total” or “complete” complementarity between nucleic acids is where each and every nucleic acid base is matched with another base under the base pairing rules. The degree of complementarity between nucleic acid strands has significant effects on the efficiency and strength of hybridization between nucleic acid strands. This is of particular importance in amplification reactions, as well as detection methods which depend upon binding between nucleic acids.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxy-ribonucleotide. The primer must be sufficiently long to prime the synthesis of extension products in the presence of the inducing agent. The exact lengths of the primers will depend on many factors, including temperature, source of primer and the use of the method.

DNA molecules are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. Therefore, an end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. As used herein, a nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements which direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, the term “an oligonucleotide having a nucleotide sequence encoding a gene” means a nucleic acid sequence comprising the coding region of a gene, i.e. the nucleic acid sequence which encodes a gene product. The coding region may be present in a cDNA, genomic DNA or RNA form. When present in a DNA form, the oligonucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

### BRIEF DESCRIPTION OF THE FIGURES

FIG. 1 presents several embodiments of a chimeric prime editor template oligonucleotide (cpetODN) comprising deoxyribonucleic acid residues and ribonucleic acid residues. Optionally, the cpetODN may include a terminal stem loop, such as an MS2 stem loop comprising ribonucleic acid as shown. Underlined residues: primer binding site. Non-underlined residues: polymerase nucleotide template. Bold/Italic: 3 nt substitution. Italic: deoxyribonucleic acid residues. Bold: ribonucleic acid residues. Asterisk: Nucleotide residues with a modification.

FIG. 1A: cpetODN #1 encoding a polymerase deoxyribonucleic acid template and a ribonucleic acid primer binding site with a terminal RNA stem loop (e.g., MS2).

FIG. 1B: cpetODN #2 encoding a polymerase deoxyribonucleic acid template and a chimeric deoxyribonucleic acid/ribonucleic acid primer binding site with a terminal RNA stem loop (e.g., MS2).

FIG. 1C: cpetODN #3 encoding a polymerase deoxyribonucleic acid template and a deoxyribonucleic acid primer binding site with a terminal RNA stem loop (e.g., MS2).

FIG. 1D. cpetODN #4 encoding a polymerase deoxyribonucleic acid template and a deoxyribonucleic acid primer binding site comprising a modification with a terminal RNA stem loop (e.g., MS2).

FIG. 1E: cpetODN #5 encoding a deoxyribonucleic acid template and a deoxyribonucleic acid primer binding site comprising a modification with a terminal RNA stem loop (e.g., MS2).

FIG. 2 presents exemplary data showing the improved nCas9 gene editing efficiencies in HEK293T cells of electroporated cpetODN constructs in comparison with conventional petRNA constructs.

FIG. 3 presents exemplary raw data of a droplet digital polymerase chain reaction (ddPCR) analysis for the data presented in FIG. 2.

FIG. 4 presents exemplary data showing equivalent nCas9 gene editing efficiencies in Moloney murine leukemia virus (MMLV) transcript with sequential transfection of cpetODN constructs in comparison with conventional petRNA.

FIG. 5 presents exemplary raw data of a droplet digital polymerase chain reaction (ddPCR) analysis for the data presented in FIG. 4.

FIG. 6 presents exemplary data showing superior nCas9 gene editing efficiency using a high fidelity Phi29 DNA polymerase with sequential transfection of cpetODNs in comparison with conventional petRNA.

FIG. 7 presents exemplary data comparing the relative gene editing efficiency of cpetODNs 1-3 with a high fidelity Phi29 DNA polymerase tethered to an nCas9 but in the absence of an MS2 coat protein (MCP).

FIG. 8 presents exemplary data of a droplet digital polymerase chain reaction (ddPCR) analysis.

FIG. 8A: Raw data plot for a 3 nt substitution probe (Channel #1) and a FANCF locus WT probe (Channel #2).

FIG. 8B: Droplet count distribution histogram showing a relatively even flow during the dd{CR analysis.

FIG. 9 presents exemplary data of deep sequencing analysis following a cpetODN gene editing experiment.

FIG. 9A: PRNP 1 nt transversion (+6 G to T)

FIG. 9B: IDS 1nt transversion (+5 G to A).

FIG. 10 presents exemplary data of the superior gene editing efficiency of the PRNP and IDS 1 nt transversions shown in in FIGS. 9A and 9B as compared to a conventional petRNA.

FIG. 11 present a summary of the above data showing the equivalent and/or superior gene editing efficiency of cpetODNs with a MCP2-PE and a high fidelity Phi29 DNA polymerase in comparison to a conventional petRNA.

FIG. 12 presents a summary of exemplary data showing a direct comparison of gene editing efficiencies between the various cpetODN constructs using either the MMLV-reverse transcriptase or the high fidelity ncoPhi29 DNA polymerase. cpetODN #2 was most efficient of the series and a dose response analysis is shown.

FIG. 13 presents exemplary data showing chimeric prime editing by M-MLV reverse transcriptase (RT) via chimeric templates.

FIG. 13A: Diagram depicting the putative mechanism of prime editing (PE) by M-MLV reverse transcriptase using chimeric linear PE templates (LPETs).

FIG. 13B: Dosage-dependent precise editing by the chimeric LPET and representative Sanger sequencing traces confirm the precise editing. The sequencing was done using the same sample from the same panel (LPET chimera 100 μmol).

FIG. 13C: Precise editing mediated by LPETs at the FANCF site (3 nt substitution) through mRNA nucleofection as measured by deep sequencing reads. Among the LPET sequences, blue, green and purple letters denote DNA, RNA and RNA with 2′-O-methylation, respectively; whereas stars denote phosphorothioate bonds. Green hairpins denote the MS2 aptamer stem loop. Editing efficiency was measured by deep sequencing reads (n=3).

FIG. 13D: Precise editing by LPET chimeras with a fully 2′-O-methylated primer binding site (PBS) plus a “−1” to “−4” positioning of a reverse transcriptase template (RTT), as measured by deep sequencing reads (n=3).

FIG. 13E: Precise editing by LPET chimeras with other different chemical modifications, including Locked Nucleic Acid (LNA), 2′ Ome, 3′ PS and 2′-F or their combinations as measured by deep sequencing reads (n=3).

FIG. 13F: Precise editing by LPET chimeras with full DNA of “RTT+PBS” plus modifications at 3 end with “no modifications” or “2-O-methyl U” or “3′ PS” as measured by deep sequencing reads (n=3).

FIG. 13G: Precise editing by LPET chimeras at PRNP and IDS gene endogenous sites as measured by deep sequencing reads.

FIG. 13H: Precise editing by LPET chimeras at PRNP and IDS gene endogenous sites as measured by deep sequencing reads.

FIG. 14 presents exemplary data showing comparing M-MLV RT and cPE-DNAPol gene editing of a 3 nt substitution in a FANCF gene with 2′-O-methylated primer binding sites.

FIG. 14A: LPET construct sequences with various chemical modifications (*) of 2′-O-methylation in the primer binding sites.

FIG. 14B: Comparative data between M-MLV PE-RT and cPE-DNAPol gene editing of a 3 nt substitution in a FANCF gene.

FIG. 14C: Annotated modification coding of fully methylated FANC primer binding sites (e.g., PBS, PBS-1, PBS-2, PBS-3 and PBS-4).

FIG. 15 presents exemplary data showing prime editing with a high fidelity DNA polymerase (e.g., Phi29).

FIG. 15A: Diagram depicting a putative mechanism of DNA polymerase editing (DPE). An MCP-tethered Phi29 DNA polymerase binds to a DNA polymerase editing template (DPET) with an MS2 aptamer. The DPET anneals to the nicked DNA strand and serves as a primer for Phi29-mediated polymerization.

FIG. 15B: Precise editing (e.g., a 3 nt substitution) at the FANCF locus by a Phi29 polymerase editor and the same panel of templates from FIG. 13c through mRNA nucleofection. The panel of DPETs are the same as in FIG. 13C. Editing efficiency was measured by deep sequencing reads (n=3).

FIG. 15C: Precise editing by DPET chimeras with a 2-O-methylated PBS plus “−1” to “−4” RTT positioning, as measured by deep sequencing reads (n=3).

FIG. 15D: Precise editing by DPET chimeras with a chemical modification, including Locked Nucleic Acid (LNA), 2′-Ome, 3′-PS and 2′-F, or their combinations, as measured by deep sequencing reads (n=3).

FIG. 15D: Precise editing by DPET chimeras with full DNA of “RTT+PBS” plus modifications at 3′ end with: i) no modifications; ii) a 2-O-methylated uracil; or iii) a 2′-O-methylated thymidine, as measured by deep sequencing reads (n=3).

FIG. 16 presents exemplary data showing precise long nucleotide editing using either a cPE-RT or cPE-DNAPol construct.

FIG. 16A: Precise editing for a simultaneous 40-bp insertion/90-bp deletion at two AA VS sites using a single LPET through mRNA nucleofection.

FIG. 16B: Deep sequencing read (n=3) verification of the data in FIG. 16A.

FIG. 16C: Illustrative diagram of a twin LPET construct for long nucleotide prime editing.

FIG. 16D: Precise editing of a 132-bp insertion at the HEK3 site. Editing efficiency was measured by ddPCR (n=2).

FIG. 17 presents exemplary data of in vivo modular prime editing performed in various cell lines comparing a cPE-RT(+2) construct with a high fidelity cPE-Phi29(Ome) construct.

FIG. 17A: MDA-MB-231 cell line.

FIG. 17B: U20S cell line.

FIG. 17C: HeLa cell line.

FIG. 17D: A549 cell line.

FIG. 18 presents exemplary data showing cPE-RT and a high fidelity cPE-NTPol modular prime editing constructs with bio-linpets.

FIG. 18A: Diagram depicting a putative mechanism of prime editing by mSA-tethered MMLV-RT or a high fidelity mSA-tethered-Phi29 polymerase via mRNA nucleofection. A biotinylated linpet (bio-linpet) is recruited by mSA and anneals to a nicked DNA for initiation of polymerization.

FIG. 18B: Precise editing (3 nt substitution) at FANCF locus by mSA-tethered MMLV-RT or a high fidelity mSA-tethered Phi29 polymerase with bio-linpet constructs.

Exemplary bio-linpet sequences. Blue Letters: DNA; Green Letters: RNA; and Purple letters: 2′-O-methylated RNA. Stars: Phosphorothioate bonds. Editing efficiency was measured by deep sequencing reads (n=3).

FIG. 19 presents exemplary data of a linpet appended with a nucleotide aptamer attached to a cPE-RT.

FIG. 19A: A modular cPE constructed with a linpet appended with a DNA aptamer that binds to an RT protein (e.g., SA-RT).

FIG. 19B: FANCF gene 3 nt insertion efficiency of three different aptamers.

FIG. 19C: An exemplary bio-linpet construct was used to compare cPEs with either an sgRNA or a sgRNA-aptamer.

FIG. 19D: The data show that the sgRNA-aptamer construct outperformed the conventional sgRNA construct whether the aptamer was DNA, RNA or a DNA/RNA chimera in the presence of the bio-linpet construct of FIG. 19C.

FIG. 20 presents exemplary data of a linpet appended with an aptamer attached to a nucleotide polymerase.

FIG. 20A: A representative diagram of a linpet appended with an MS2 aptamer attached to a DNA polymerase associated with a cPE (MCP-Pol15M).

FIG. 20B: Representative data showing MCP-Pol15M prime editing activity with a non-methylated linpet-chimera (+2) primer binding site.

FIG. 20C: Representative data showing MCP-Pol15M prime editing activity with a fully methylated linpet-chimera (+2) primer binding site.

FIG. 21 presents exemplary data of prime editing with chimeric linear prime editing templates (LPETs).

FIG. 21A: Diagram depicting the putative mechanism of LPET editing. MCP-tethered RT binds to LPET via an MS2 stem-loop. The LPET anneals to the nicked DNA strand and serves as template for RT.

FIG. 21B: Dose-dependent precise editing (deep sequencing) with the LPET (+0 configuration, see below) at the FANCF editing site. HEK293T cells were electroporated with indicated mRNA (1 μg), sgRNA (100 pmol), nicking sgRNA (100 pmol), and LPET.

FIG. 21C: Precise editing mediated by LPET (+2 configuration, see below), MS2-less LPET(+2) (with end modification), MS2-less ssDNA LPET (without end modification), linear LPET(−17) with end protection, and unmodified, all-RNA LPET (without end modification).

FIG. 21D: Precise editing mediated by LPETs with different DNA replacements at the FANCF site (3 nt substitution, denoted by bold letters). Left: template sequences (edited bases in bold). The “sgRNA only” sample was cells only treated with sgRNA as a negative control. Stars denote phosphorothioate bonds. Green hairpins denote MS2. Right: editing efficiency was measured by deep sequencing on day 3 (n=3). Grey bars denote indels.

FIG. 21E: Precise editing by LPETs with fully 2′-O-methyl PBS (+0) or with “+1” to “+4” DNA, as measured by deep sequencing (n=3) (hereinafter referred to as Ome LPET).

FIG. 21F: Precise editing by LPET (+0) and unmodified LPET at IDS endogenous sites. Data and error bars indicate the mean and s.d. of three independent biological replicates. two-tailed unpaired Student's t-test: *, P<0.05; **, P<0.01; ***, P<0.001; ****, P<0.0001.

FIG. 21G: Precise editing by LPET (+0) and unmodified LPET at PRNP endogenous sites. Data and error bars indicate the mean and s.d. of three independent biological replicates. two-tailed unpaired Student's t-test: *, P<0.05; **, P<0.01; ***, P<0.001; ****, P<0.0001.

FIG. 22 presents exemplary data of LPET mediated precision editing.

FIG. 22A: Diagram of LPET, MS 2-less LPET, MS 2-less ssDNA, modified petRNA (17 and unmodified petRNA.

FIG. 22B: LPET and no RT controls HEK 293 T cells were electroporated with indicated mRNA (1 μg), sgRNA (100 μmol), nicking sgRNA (100 μmol) and FANCF (LPET 2+), PRNP (LPET 0+), IDS (LPET 0+), RUNX (1+), LPET (2+), HBB (LPET 2+). Editing was measured by deep sequencing n=3. Data and error bars indicate mean and s. d. of three independent biological replicates

FIG. 22C: Representative reads for FANCF and PRNP. Some small deletions at FANCF are likely caused by short homology near the nicking sites. Top five reads are shown.

FIG. 23 presents exemplary data of MMLV RT mediated precision editing with different LPET modifications.

FIG. 23A: Various modifications in the LPET. Precision editing by LPET with additional chemically modified residues including Locked Nucleic Acid (LNA, IN), 2′-F, 2′-O-me (mN) and 3-phosphorothioate (PS) as measured by deep sequencing in 293T cells (n=3). rN=RNA; *=3′ PS linker. All sequences are written from 5′ to 3′. Data and error bars indicate the mean and s. d. of three independent biological replicates.

FIG. 23B: RT mediates precise editing by LPETs with an all-DNA RTT and all-DNA PBS with no modifications, or with the indicated 3′-terminal modifications, as measured by deep sequencing (n=3). Blue and purple letters denote DNA, RNA and 2-O-methyl RNA, respectively.

FIG. 24 presents exemplary data showing high fidelity DNA polymerase gene editing. Gene editing efficiency was measured by deep sequencing on day 3 (n=3).

FIG. 24A: Diagram depicting a putative mechanism of HFPhi29Pol DNA polymerase editing (DPE). MCP-tethered HFPhi29Pol binds to a DPE template (e.g., DPET) via MS2. The template anneals to the nicked DNA strand and serves as the template for polymerization and insertion.

FIG. 24B: Precise editing (e.g., a 3 nt substitution) at the FANCF locus by HFPhi29Pol and the templates shown in FIG. 21C via mRNA nucleofection. Grey bars denote indels.

FIG. 24C: Precise editing by DPETs with fully 2′-O-methylated PBS (+0) or with “+1” to “+4” DNA as shown in FIG. 21D. The “no polymerase control” was treated with Cas9 nickase mRNA, sgRNA, nk-sgRNA, and DPET(+2).

FIG. 24D: FANCF DPET(+2), PRNP DPET(+0), HBB DPET(+2), and IDS DPET(+0) were used with HFPhi29Pol, and as LPETs used with RT, in the MDA-MB-231 cell line.

FIG. 24E: Gene editing with LPET/DPET(+0) in human IMR90 primary lung fibroblasts at the PRNP and IDS loci.

FIG. 24F: LPET/RT- or DPET/HFPhi29Pol-based editing restored mCherry expression by a 4 nt insertion in a reporter cell line. mCherry was quantified by flow cytometry (n=3).

FIG. 24G: Representative LPET/RT FACS plot of data in FIG. 24F. For all panels, cells were electroporated with indicated mRNA (1 μg), sgRNA (100 μmol), nicking sgRNA (100 μmol), and LPET or DPET (100 μmol). Data and error bars indicate mean and s.d. of three independent biological replicates.

FIG. 24H: Representative DPET/HFPhi29Pol FACS plot of data in FIG. 24F. For all panels, cells were electroporated with indicated mRNA (1 μg), sgRNA (100 μmol), nicking sgRNA (100 μmol), and LPET or DPET (100 μmol). Data and error bars indicate mean and s.d. of three independent biological replicates.

FIG. 25 presents exemplary data showing HFPhi29Pol gene editing with unmodified and modified DPETs.

FIG. 25A: Precise editing mediated by HFPhi29Pol. HEK293T cells were electroporated with: i) mRNA (1 μg), sgRNA (100 μmol), nicking sgRNA (100 μmol); and ii) DPET(+0), MS2-less DPET, MS2-less single stranded DNA (RTT+PBS), modified linear DPET(−17) or unmodified (e.g., all-RNA) DPET.

FIG. 25B: Precise editing mediated by HFPhi29Pol and pegRNA. Editing efficiency was measured by deep sequencing (n=3). Data and error bars indicate the mean and s. d. of three independent biological replicates two tailed unpaired Student's t test: * P<0.05; ** P<0.01; *** P<0.001; **** P<0.0001.

FIG. 26 presents exemplary data showing HFPhi29Pol DPE with different DPET modifications.

FIG. 26A: HFPhi29Pol-mediated precision editing with various modifications including LNA, 2′-F, 2′-O-me, and 3′-PS in the DPET as measured by deep sequencing (n=3). IN=Locked Nucleic Acid, rN=RNA. mN=2′Ome, rN=RNA, *=3′ PS linker. All sequences are written from 5′ to 3′. Data and error bars indicate the mean and s. d. of three independent biological replicates.

FIG. 26B: HFPhi29Pol mediates precise editing by DPETs with an all-DNA RTT and PBS with: i) no modifications; or ii) the indicated 3′ terminal modifications, as measured by deep sequencing (n=3). Blue and purple letters denote DNA, RNA and 2′-O-methyl RNA residues, respectively.

FIG. 27 presents exemplary data showing incremental DNA replacement in the PBS/RTT targeted to a PRNP site. Precision editing with DNA replacement in the PBS/RTT with LPET/RT (left panel) or DPET/HFPhi29Pol (right panel). Data and error bars indicate the mean and s.d. of three independent biological replicates.

FIG. 28 presents exemplary data showing that LPETs and DPETs result in precise genome editing without an additional nicking sgRNA in multiple endogenous sites.

FIGS. 28 A-C: HEK293T cells were electroporated with indicated mRNAs (1 μg), pegRNA (100 μmol, PE2) and nicking sgRNA (100 μmol, PE3). LPET and DPET groups include mRNAs, sgRNA and nicking sgRNA (100 μmol) and LPET/DPET (FANCF-Chimera(+2); PRNP-Chimera(+0); RUNX1 Chimera(+2). Data and error bars indicate the mean and s. d. of three independent biological replicates.

FIG. 29 presents exemplary data showing that biotinylated LPETs and DPETs result in precise genome editing.

FIGS. 29A-C: LPET/RT and DPET/HFPhi29Pol editing in multiple cell lines, as measured by deep sequencing (n=3). ND, not determined. “SNP” indicates the existence of a SNP in the HBB target region in the U2 OS cell line, precluding comparative testing

FIG. 29D: Biotinylated LPETs and biotinylated DPETs in an mCherry reporter line (left panel) and at endogenous sites (right panel) (n=3). Data and error bars indicate the mean and s. d. of three independent biological replicates. FIG. 30 presents exemplary data showing precision genome editing using monomeric streptavidin (mSA)-tethered polymerases and biotinylated templates.

FIG. 30A: Diagram depicting mSA-RT and HFPhi29Pol. The 5′-biotinylated LPET or DPET is recruited by mSA and anneals to the nicked DNA.

FIG. 30B: Biotinylated LPET/DPET with different modification patterns. Asterisks denote phosphorothioate linkages.

FIG. 30C: Precise editing (e.g., 3 nt substitution) at the FANCF locus by mSA-RT/bio-LPET or mSA-Phi29/bio-DPET. HEK293T cells were electroporated with indicated mRNA (1 μg), sgRNA (100 μmol), nicking sgRNA (100 μmol), and LPET or DPET (100 μmol). The “sgRNA only” sample was from cells only treated with sgRNA as a negative control. Editing efficiency was measured by deep sequencing (n=3). Data and error bars indicate the mean and s.d. of three independent biological replicates.

FIG. 31 presents exemplary data showing PoI15M DNA polymerase, MarathonRT and TGIRT gene editing with synthetic templates.

FIG. 31A: Diagram depicting PoI15M/DPET gene editing (MCP-tethered PoI15M binds to the DPET via MS2. The DPET anneals to the nicked DNA strand and serves as the template for PoI15M DNA polymerase. Data indicate precision editing (e.g., a 3 nt substitution) at the FANCF locus with DPET (+2) and fully Ome-substituted PBS (right).

FIG. 31B: Precise editing at the FANCF locus with MarathonRT and TGIRT Editing efficiency was measured by deep sequencing on day 3 (n=3). Data and error bars indicate the mean and s. d. of three independent biological replicates

FIG. 32 presents exemplary data showing off-target and cytotoxicity evaluations.

FIG. 32A: On-target and off-target editing efficiencies for FANCF. Data and error bars indicate the mean and standard deviation of three independent biological replicates.

FIG. 32B: HEK293T cells were electroporated with the indicated mRNA reagents or buffers. Cell viability was measured by the CellTiter-Glo assay (n=3) on the indicated time points. Data and error bars indicate mean and s.d. of three independent biological replicates. Two-tailed unpaired Student's t-test: *, P<0.05.

FIG. 33 presents exemplary data showing precise editing of a 40 bp replacement and a 132 bp insertion.

FIG. 33A: Precise editing for a 40 bp insertion and 90 bp deletion at two AAVS sites (e.g., AAVS 1615 and AAVS 1705) using LPET/DPET through mRNA nucleofection, followed by PCR amplification and agarose gel electrophoresis.

FIG. 33B: Quantification of the 40 bp insertion of FIG. 33A by deep sequencing (n=3). Data and error bars indicate the mean and s. d. of three independent biological replicates, unless otherwise indicated.

FIG. 33C: Diagram of the TwinPE system by LPETs/DPETs for a long template insertion.

FIG. 33D: Precise editing for a 132 bp insertion at the HEK3 site. Editing efficiency was measured by ddPCR (n=2).

FIG. 34 presents exemplary template sequences.

FIG. 34A: MCP-M_MLV RT

FIG. 34B: mSA-RT

FIG. 34C: MCP-HFPhi29Pol

FIG. 34D: MCP-PoI15M

FIG. 34E: mSA-HFPhi29Pol

### DETAILED DESCRIPTION OF THE INVENTION

The present invention relates to the field of genomic engineering. In particular, a chimeric prime editing (cPE) system is disclosed comprising elements including, but not limited to a Cas9 nickase (nCas9)/high fidelity nucleotide polymerase (HFNTPol), a single guide RNA (sgRNA), a chimeric prime editor template oligonucleotide (cpetODN) comprising a deoxyribonucleic acid nucleotide polymerase template (NPT) and a primer binding site. For example, an sgRNA and the cpetODN are ligated into a single pegRNA. Alternatively, an sgRNA and the cpetODN are free and independent molecules (e.g., modular). This cPE system results in precise and efficient genome editing in cells and in adult mouse liver which is advantageous over conventional prime editor fusion constructs with pegRNAs and lower-fidelity reverse transcriptase. This flexible and modular system is an improvement in the art to obtain precise genome editing.

### I. Conventional Prime Editor Systems

Prime editors enable deletion, insertion, and base substitution without double-strand breaks. Anzalone et al., “Search-and-replace genome editing without double-strand breaks or donor DNA” Nature 576:149-157 (2019). However, this known fusion of a Cas9 nickase (nCas9; PE2) and a Moloney murine leukemia virus reverse transcriptase (M-MLV RT)) is >6.3 kb. This size is beyond the packaging capacity of a single adeno-associated virus (AAV).

Production of such a large protein in recombinant form in high yield to accommodate ribonucleoprotein (RNP) delivery can also be challenging. Some split Cas9 fusion construct strategies have been tested for the delivery of genome editing tools, including split inteins and MS2 or SunTag tethers. However, most of those split Cas9 fusion construct approaches have not yet been applied to prime editors. Wang et al., “CRISPR-Based Therapeutic Genome Editing: Strategies and In Vivo Delivery by AAV Vectors” Cell 181:136-150 (2020): Truong et al., “Development of an intein-mediated split-Cas9 system for gene therapy” Nucleic Acids Res 43:6450-6458 (2015); Maji et al., “Multidimensional chemical control of CRISPR-Cas9” Nat Chem Biol 13:9-11 (2017)” Liu et al., “A chemical-inducible CRISPR-Cas9 system for rapid control of genome editing” Nat Chem Biol 12:980-987 (2016): Li et al., “SWISS: multiplexed orthogonal genome editing in plants with a Cas9 nickase and engineered CRISPR RNA scaffolds” Genome Biol 21:141 (2020): Konermann et al., “Genome-scale transcriptional activation by an engineered CRISPR-Cas9 complex” Nature 517:583-588 (2015)” Wang et al., “sgBE: a structure-guided design of sgRNA architecture specifies base editing window and enables simultaneous conversion of cytosine and adenosine” Genome Biol 21:222 (2020); Jiang et al., “BE-PLUS: a new base editing tool with broadened editing window and enhanced fidelity” Cell Res 28:855-861 (2018).

These previously reported PE systems may also include a conjugated RNA that consists of a single guide RNA (sgRNA), a 3′ extension containing the reverse transcriptase (RT) template (RTT) nucleotide and a primer binding site (PBS), referred to herein as a prime editor guide RNA (e.g., pegRNA). Despite their usefulness, such pegRNAs are prone to misfolding due to inevitable inappropriate base pairing between the PBS and a spacer, as well as potential RTT-scaffold binding interactions. Finally, the 3′-terminal extension in the pegRNA is exposed to the cytosol and is therefore susceptible to degradation by nucleases, which may compromise the integrity of the pegRNA. Therefore, efforts to reduce pegRNA misfolding and instability are needed.

### II. Conventional Split and Modular Prime Editor Constructs

Previously reported split prime editor fusion constructs include, but are not limited to, an MS2-PE2 and SunTag-PE2 fusion constructs. MS2-PE2 comprises an MS2 coat protein (MCP) fused to the N-terminus of an M-MLV RT protein. Multiple cognate MS2-pegRNAs were engineered by incorporating MS2 stem-loops into different positions of the sgRNA. Additionally, a split SunTag fusion construct was created by fusing an scFv protein fragment to an N-terminus of M-MLV RT protein. Subsequently, the SunTag scFv-RT fusion construct was recruited by either GCN4-nCas9 or nCas9-GCN4. These two PE2 fusion constructs are generally referred to as SunTag-PE2 (GCN4-nCas9) and PE2-SunTag (nCas9-GCN4) based on domain element order.

The MS2, SunTag and sPE platforms have been designated in the art as a prime editor 3 (PE3) format. The PE3 format differs from PE2 by inclusion of an additional sgRNA that directs nicking of the unedited strand, thereby biasing repair. The respective nCas9-, RT-, pegRNA-, and nicking sgRNA-expressing plasmids were co-transfected into a HEK293T-derived mCherry reporter lentivector-transduced cell line with a premature TAG stop codon that can be reverted to wild type codon, yielding a red fluorescence signal. Liu et al., “Improved prime editors enable pathogenic allele correction and cancer modelling in adult mice” Nat Commun 12:2121 (2021). The most potent MS2- and SunTag-tethered configurations were comparable in editing efficiency to a PE3 fusion construct.

III. Chimeric Prime Editor Systems (cPE)

In one embodiment, the present invention contemplates a chimeric prime editor system (cPE) comprising a nucleotide polymerase. In one embodiment, the nucleotide polymerase is a DNA polymerase. In one embodiment, the nucleotide polymerase is a reverse transcriptase. In one embodiment, the chimeric prime editor system comprises a chimeric prime editor template oligonucleotide (cpetODN) that is at least partially a deoxyribonucleic acid sequence. In one embodiment, the cpetODN comprises at least one ribonucleic acid sequence. In one embodiment, the cpetODN is a deoxyribonucleic acid sequence. In one embodiment, the cpetODN further comprises a stem loop. In one embodiment, the stem loop is an MS2 stem loop.

In one embodiment, the present invention contemplates a Cas9 chimeric prime editor system comprising elements including, but not limited to, a fusion protein comprising an nCas9/sgRNA complex and a high fidelity nucleotide polymerase, a ribooligonucleotide comprising a primer binding site (PBS) ligated to a nucleotide polymerase template (NPT). In one embodiment, the system comprises at least one vector encoding: i) a fusion protein comprising an nCas9 and a high fidelity nucleotide polymerase; ii) an sgRNA; and iii) an oligonucleotide comprising a primer binding site and a nucleotide polymerase template. In one embodiment, the system comprises a first vector encoding an sgRNA and a fusion protein comprising an nCas9 and high fidelity nucleotide polymerase; and a second vector comprising a ribooligonucleotide encoding a primer binding site and a nucleotide polymerase template. In one embodiment, the second vector further comprises a stem loop oligonucleotide.

A. Chimeric Prime Editor Template Oligonucleotides (cpetODNs)

Those in the art have recognized that obstacles exist to permit prime editing to efficiently edit nucleic acids of greater length than the typical <100 nt insertions. One limitation on conventional prime editor length is that reverse transcriptases tend to have modest processivity (e.g., how far they can polymerize before falling off) and are also limited in terms of fidelity. For example, such low fidelity polymerases incur errors made during polymerization occur in such a way that cause mutations. Error-induced mutation is thus a problem that gets ever worse with increasing length of polymerization, and with increasing numbers of cells to be independently edited. As such, the high fidelity nucleotide polymerases, as disclosed herein, solve this mutation problem by having a reduced error rate as compared to conventional (e.g., low fidelity) polymerases that increase misincorporation frequencies, especially when insertion lengths are >100 nts.

In one embodiment, the present invention contemplates a PE system that replaces the conventional reverse transcriptase with a high fidelity nucleotide polymerase, including but not limited to a high fidelity DNA polymerase or a high fidelity reverse transcriptase. Preferably, the invention contemplates that a high fidelity nucleotide polymerase is a processive DNA polymerase or a processive reverse transcriptase that comprises a low error rate (e.g., <10′ nts) to minimize errors and subsequent mutations. In one embodiment, a high fidelity processive DNA polymerase is a phage DNA polymerase (e.g., Phi29). In one embodiment, a processive DNA or a reverse transcriptase inserts a nucleotide template of >100 nts.

Those in the art have not considered that a prime editor system utilizing a processive DNA polymerase or a processive reverse transcriptase as an improvement in the art of gene editing. The use of DNA polymerases in CRISPR systems was reported in an effort to increase the number of mutations. Halperin et al., “CRISPR-guided DNA polymerases enable diversification of all nucleotides in a tunable window” Nature 560:248-252 (2018); and Tou et al., “Targeted Diversification in the S. cerevisiae Genome with CRISPR-Guided DNA Polymerase I” ACS Synth. Biol. 9:1911-1916 (2020) These objectives are opposite that contemplated herein, to precisely edit a genome with the correct nucleotide sequence.

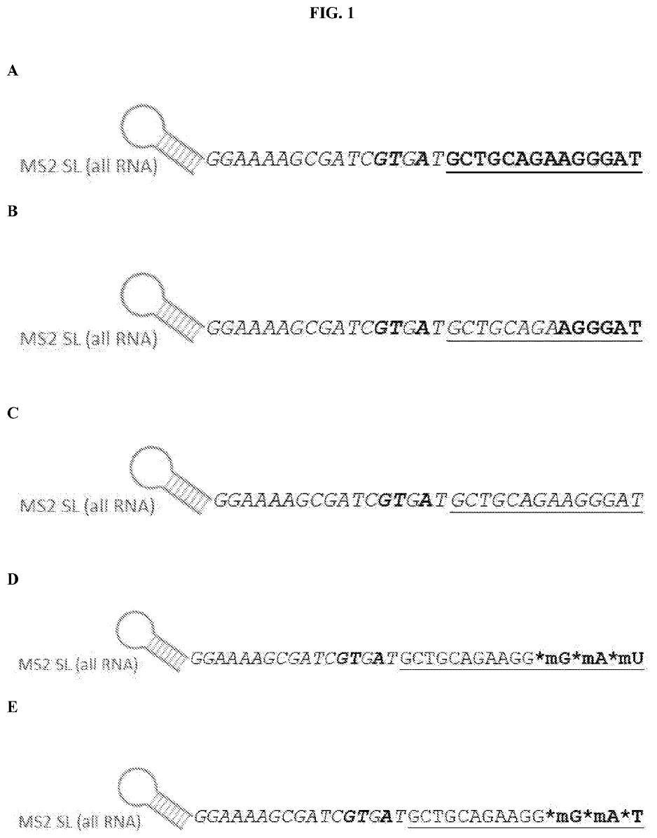

In one embodiment, a chimeric PE system (cPE) comprises a hybrid oligonucleotide encoding a nucleotide polymerase template oligonucleotide and a primer binding site (e.g., a cpetODN). In one embodiment, the hybrid oligonucleotide comprises a ribonucleic acid region and a deoxyribonucleic acid region. In one embodiment, the primer binding site is encoded by a ribonucleic acid (cpetODN #1). See, FIG. 1A. In one embodiment, the primer binding site is encoded by a deoxyribonucleic acid (cpetODN #2). See, FIG. 1B. In one embodiment, the primer binding site is encoded by both deoxyribonucleic acids and ribonucleic acids (cpetODN #3-#5). See, FIGS. 1C-1E. In one embodiment, the cpetODN further comprises an ribonucleic acid stem loop. In one embodiment, the stem loop is an MS2 stem loop. See, FIGS. 1A-E.

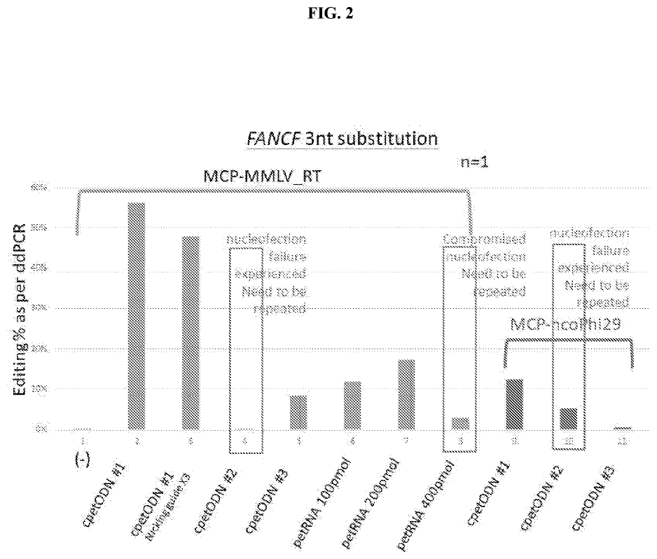

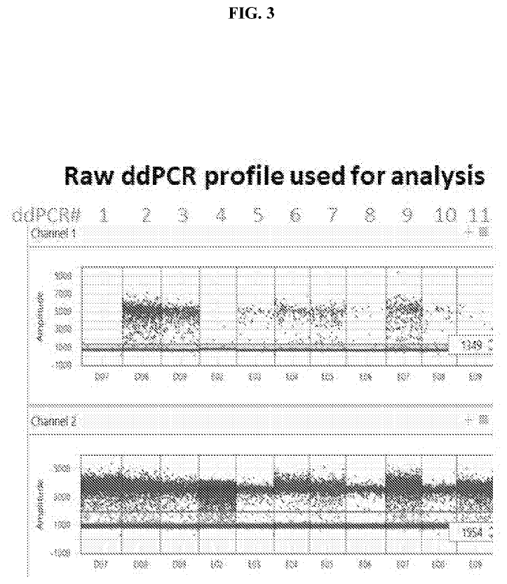

The gene editing efficiencies of cpetODN #1, cpetODN #2 and cpetODN #3 were compared to different concentrations of conventional petRNAs in HEK293T cells. All cpetODNs were co-electroporated into HEK293T cells with 200 pmol sgRNA, 60 pmol nicking guide, 1 μg H840A mRNA and 1 μg MCP-RT/1.4 μg MCP-ncoPhi29 and incubated for three (3) days before gDNA harvest. The data show that cpetODN #1 has an approximate 3-4 fold superior gene editing efficiency than any of the conventional petRNA constructs. See, FIG. 2. The raw data of the droplet digital polymerase chain reaction analysis for the results of FIG. 1 are provided. See, FIG. 3.

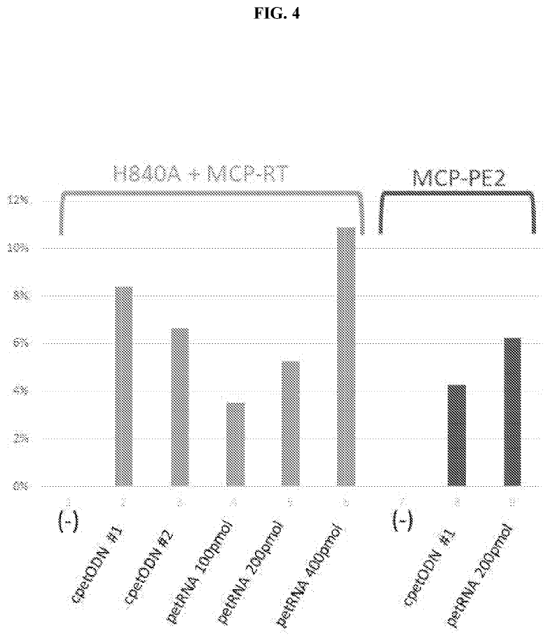

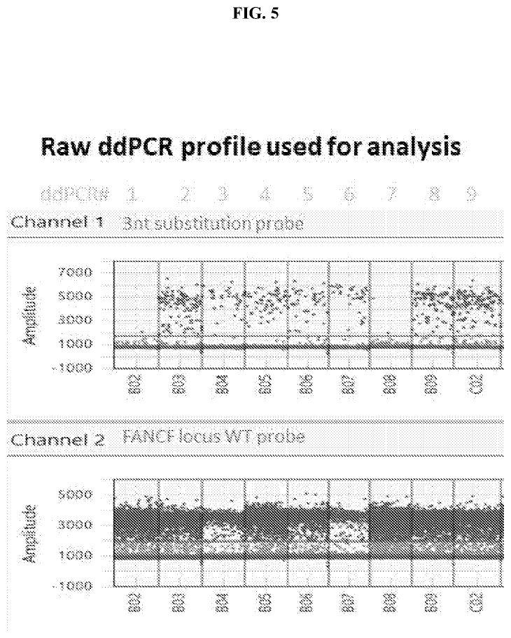

The gene editing efficiencies of cpetODN #1 and cpetODN #2 were compared to different concentrations of conventional petRNAs using an MMLV transcript as a nucleic acid. nCas9^{H840A}, MCP-RT or MCP-PE2, sgRNA and nicking guide plasmids were lipofected 24 h prior to sequential cpetODN nucleofection. The data show that the cpetODN constructs had similar gene editing efficiencies as the petRNA positive controls. Notably, the gene editing efficiencies of the cpetODN constructs were superior to the two lower petRNA concentrations. See, FIG. 4. The raw data of the droplet digital polymerase chain reaction analysis for the results of FIG. 4 are provided. See, FIG. 5.

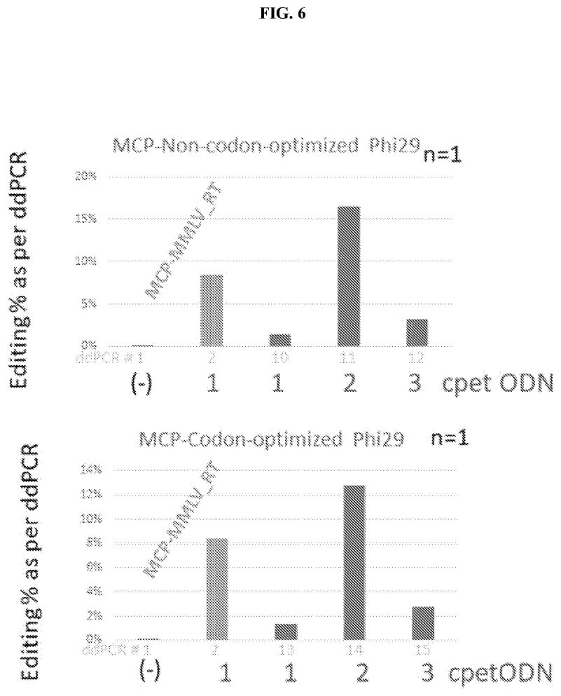

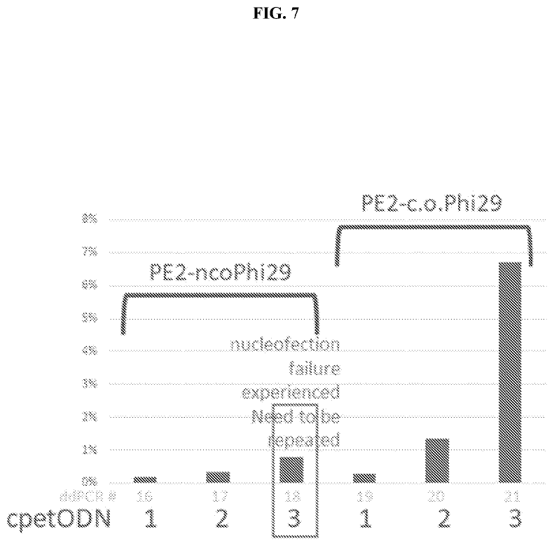

The gene editing efficiencies of cpetODN #1, cpetODN #2 and cpetODN #3 were compared to a conventional petRNA (MCP-MMLV_RT) using a high fidelity Phi29 DNA polymerase. nCas9^{H840A}, MCP-reverse transcriptase or MCP-HFPhi29Pol, sgRNA and nicking guide plasmids were lipofected 24 h prior to sequential cpetODN nucleofection. cpetODN #2 showed superior gene editing efficiency when using the Phi29 DNA polymerase was either non-codon optimized (nco) or codon-optimized (c.o.) as compared to the conventional petRNA. See, FIG. 6. A further experiment compared a cPE-HFPhi29Pol fusion protein (e.g., nco versus c.o.), wherein the high fidelity Phi29 DNA polymerase was tethered to the nCas9 but without MCP. These data show a progressive increase in gene editing efficiency of the cpetODNs as the amount of deoxyribonucleotides increase in the primer binding site (cf, cpetODN #1<cpetODN #2<cpetODN #3). See, FIG. 7. Raw data of a droplet digital polymerase chain reaction (ddPCR) analysis is presented for a 3 nt substitution probe (Channel #1) and a FANCF locus WT probe (Channel #2). See, FIG. 8A. Droplet count distribution histogram showing a relatively even flow during the ddPCR analysis. See, FIG. 8B.

Deep sequencing analysis was performed following a cpetODN gene editing experiment demonstrating that the gene editing is precise. First, a PRNP gene 1 nt transversion (+6 G to T) showing:

GCAGTGGTGGGGGGCCTTGGCGGCTACATG

[symbol]

GCAGTGGTGGGGGGCCTTGGCGTCTACATG

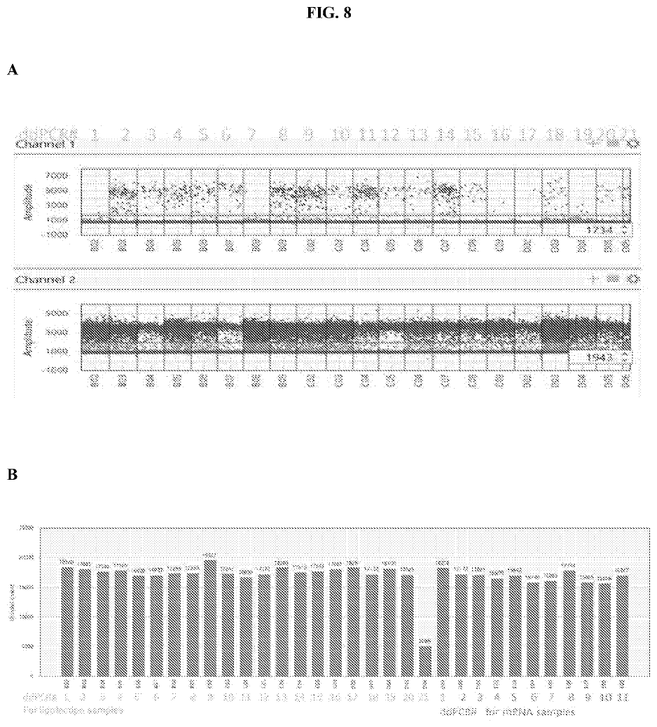

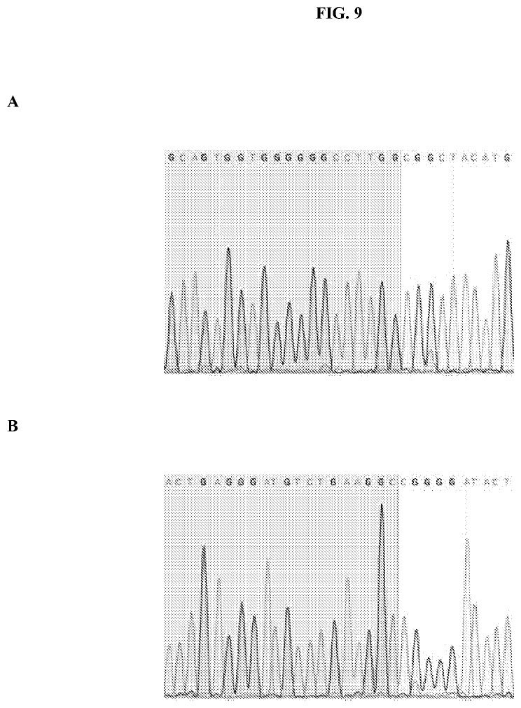

See, FIG. 9A. Second, an IDS gene 1 nt transversion (+5 G to A) showing:

ACTGAGGGATGTCTGAAGGCCGGGGATACT

[symbol]

ACTGAGGGATGTCTGAAGGCCAGGGATACT

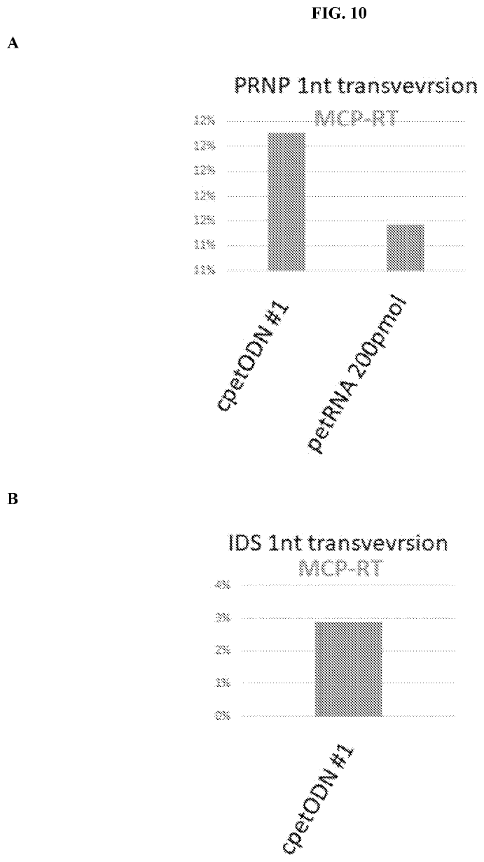

See, FIG. 9B. The gene editing efficiency of these data demonstrates superiority of the cpetODN construct as compared to a conventional petRNA. See, FIG. 10.

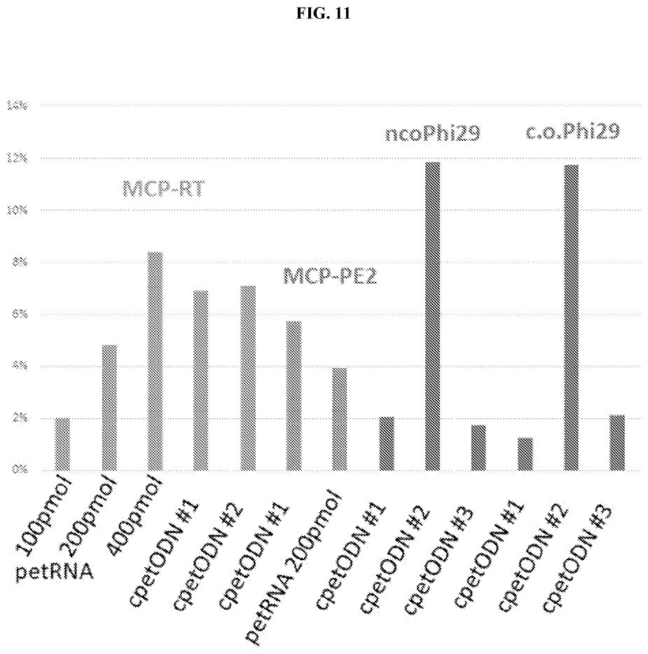

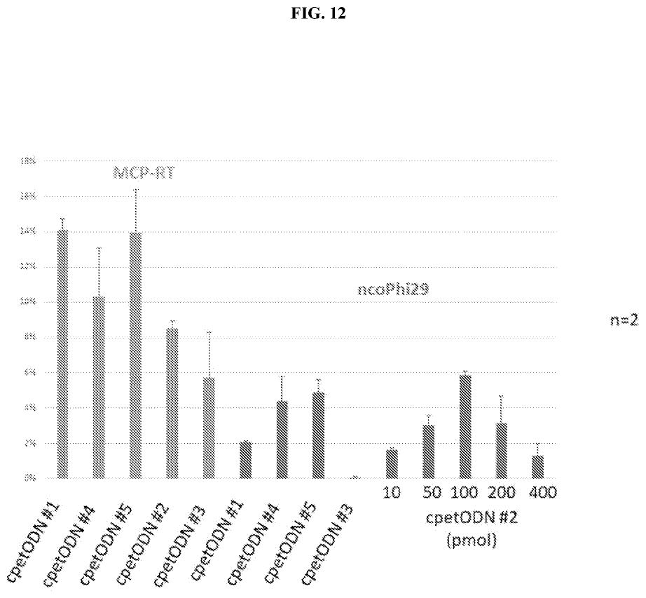

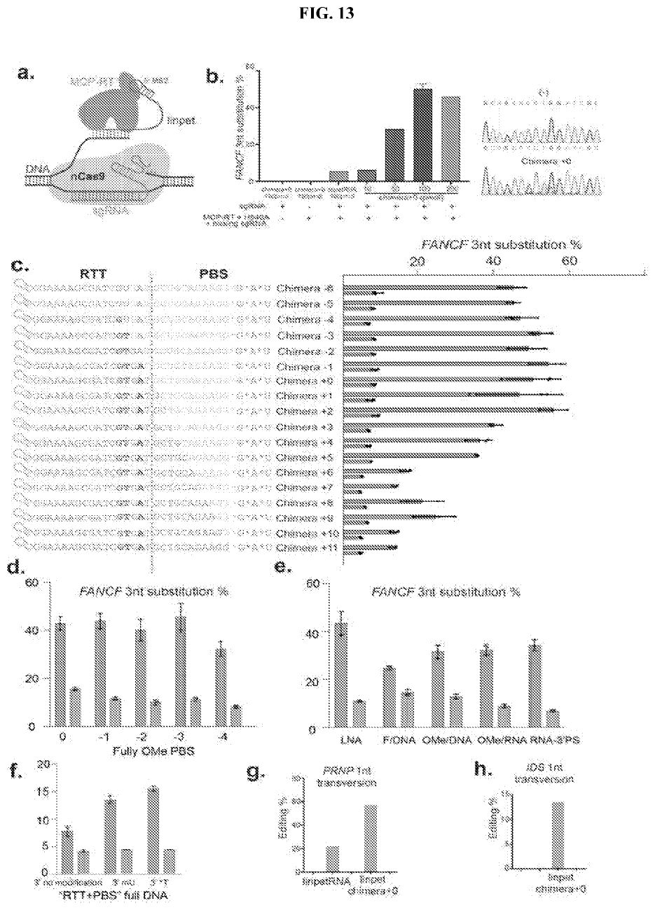

A summary of the above data for the equivalent and/or superior gene editing efficiency of cpetODNs in the MCP2-PE, c.o.HFPhi29Pol and ncoHFPhi29Pol data in comparison to a conventional petRNA is shown. See, FIG. 11. A direct comparison of the gene editing efficiencies of the various cpetODN constructs were compared between the MMLV-reverse transcriptase and a high fidelity ncoHFPhi29 DNA polymerase. cpetODN #2 was most efficient of the series and a dose response analysis is shown. See, FIG. 12.

### IV. Modular cPE Systems

Unlike the above-described conventional split PE (sPE) fusion constructs, a modular cPE system can result in versatile and efficient somatic genome editing and may include, but is not limited to, either a chimeric petODN or a petRNA in conjunction with a nucleotide polymerase. In a conventional split PE fusion construct, the entire PE system exists as a single molecule. In contrast, a modular PE system provides the PE system in physically separate elements. The present invention is not limited to the description below exemplifying a modular PE system with a cpetODN or a petRNA and a nucleotide polymerase.

The data provided herein demonstrate that a modular cPE system provides equal or superior gene editing efficiencies when compared to a modular RT-sPE systems. For example, a chimeric prime editor (cPE) system has comparable or superior gene editing with a conventional sPE, PE2 and/or split PE2/3 fusion constructs. In addition, reduction in the molecular size of either a Cas9 nickase or an NPT or RPT, as compared to a split-PE2 fusion construct may improve the yields of mRNA and/or protein production for nonviral delivery.

The data presented herein indicates that free and independent DNAPol elements can successfully engage a RNA-DNA hybrid at a Cas9 nicking site without a direct fusion to Cas9. Although it is not necessary to understand the mechanism of an invention, it is believed that an untethered RT or DNAPol is recruited to a RNA-DNA hybrid at a nick created by an sgRNA or a pegRNA. Despite recent reports addressing PE off-target effects, it is further believed that tethered or untethered RT or DNAPol could process endogenous RNA-RNA or RNA-DNA hybrids and induce potential genomic integration events. Jin et al., Genome-wide specificity of prime editors in plants. Nat Biotechnol, 319 (2021); and Zhang, L. et al

In one embodiment, use of a prime editing template ribonucleic acid (petRNA) or a chimeric prime editing oligonucleotide (cpetODN) allows programmable, high efficiency editing at a given nicking site. In one embodiment, the petRNA or cpetODN encodes a primer binding site. In one embodiment, the petRNA encodes an NPT. In one embodiment, the cpetODN comprises a chimeric NPT. In one embodiment, the petRNA or cpetODN is circular. In one embodiment, the petRNA or cpetODN is linear.

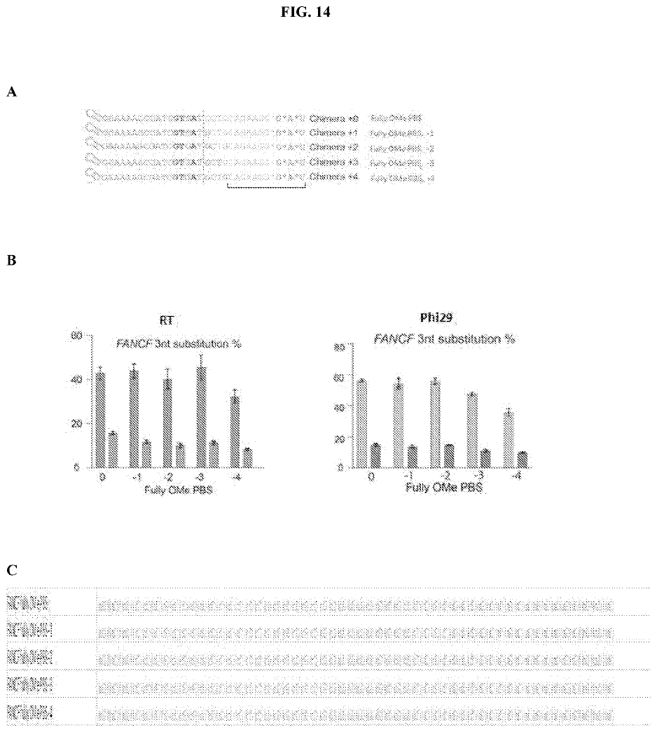

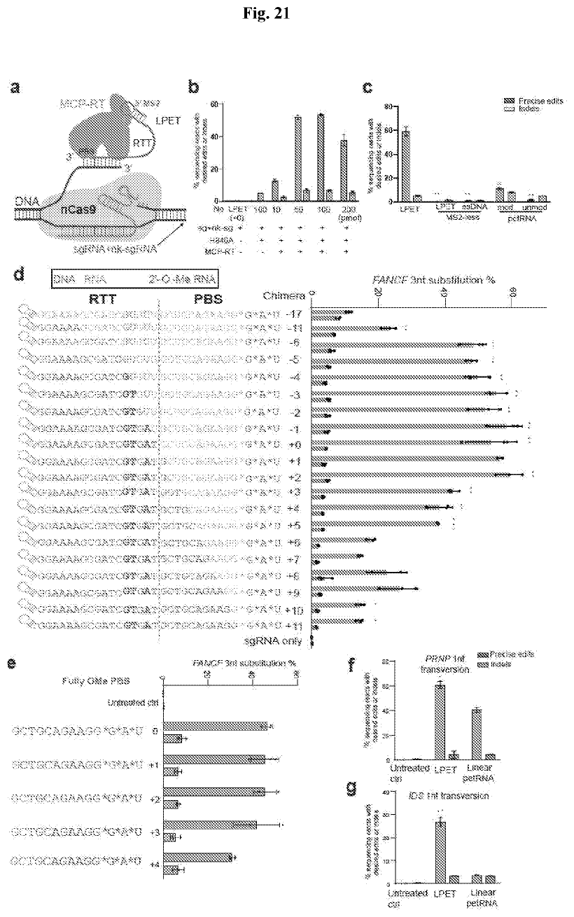

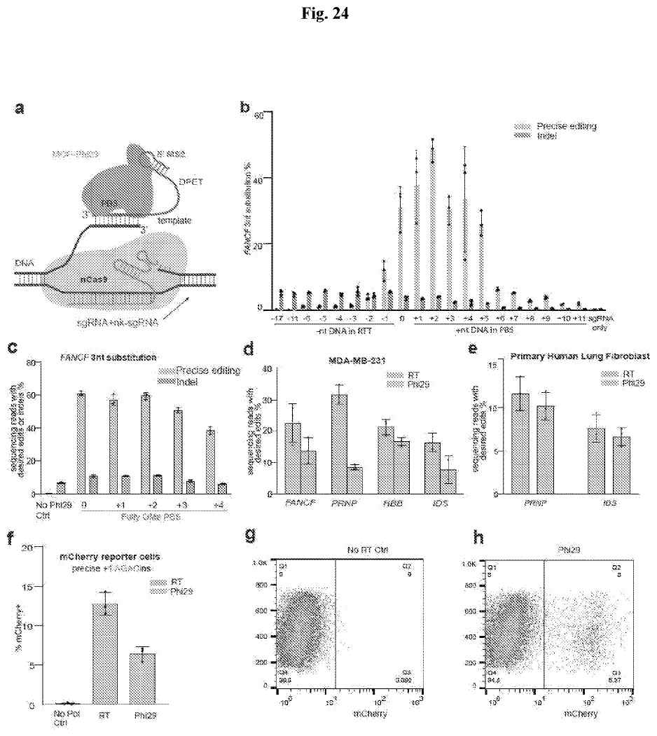

Modular chimeric prime editing may be performed with precision using elements including, but not limited to: i) synthetic linear chimeric templates (linpets or LPETS); ii) nucleotide polymerases; and/or iii) nucleotide polymerase templates. Even so, chimeric prime editing with chimeric linpets has been shown to be precise when using conventional M-MLV reverse transcriptase (RT). See, FIGS. 21A-G. Further, conventional PE systems with an M-MLV RTT was directly compared to that of cPE-DNAPolT prime editing. These data show comparable gene editing of a 3 nt insertion into a FANCF gene with either an M MLV-PE-RTT or a cPE-DNAPolT construct. See, FIGS. 14A-C. Similarly, precision prime editing using cPE with a DNAPol (e.g., HFPhi29Pol) was observed when using a variety of DPET constructs. See, FIGS. 24A-H.

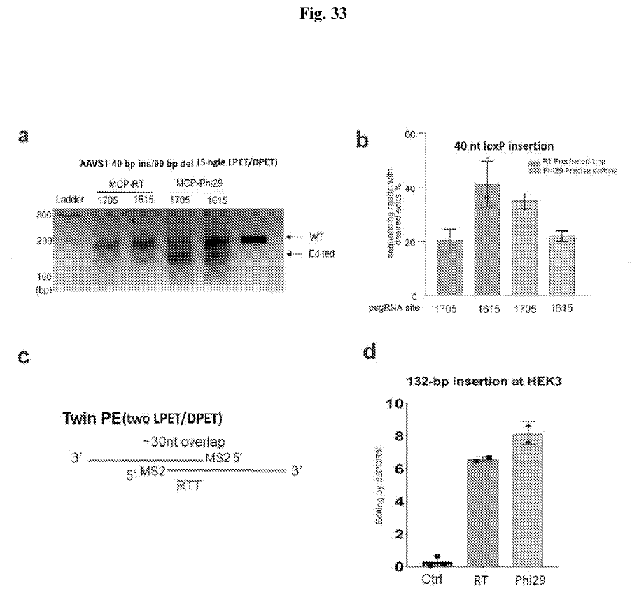

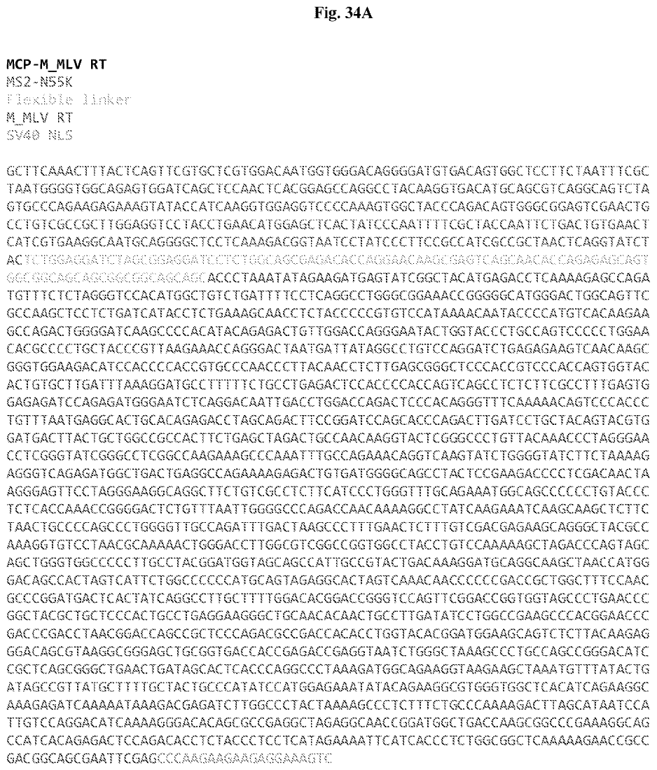

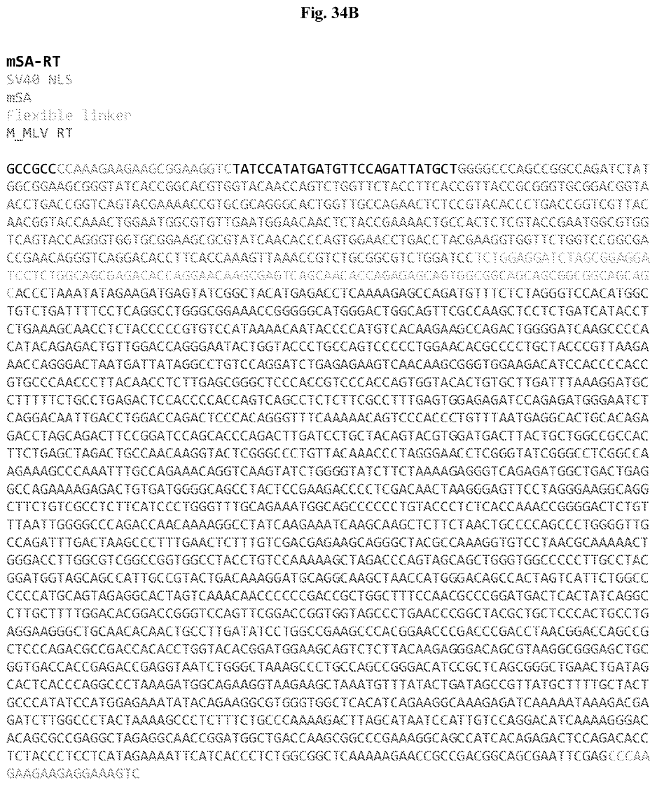

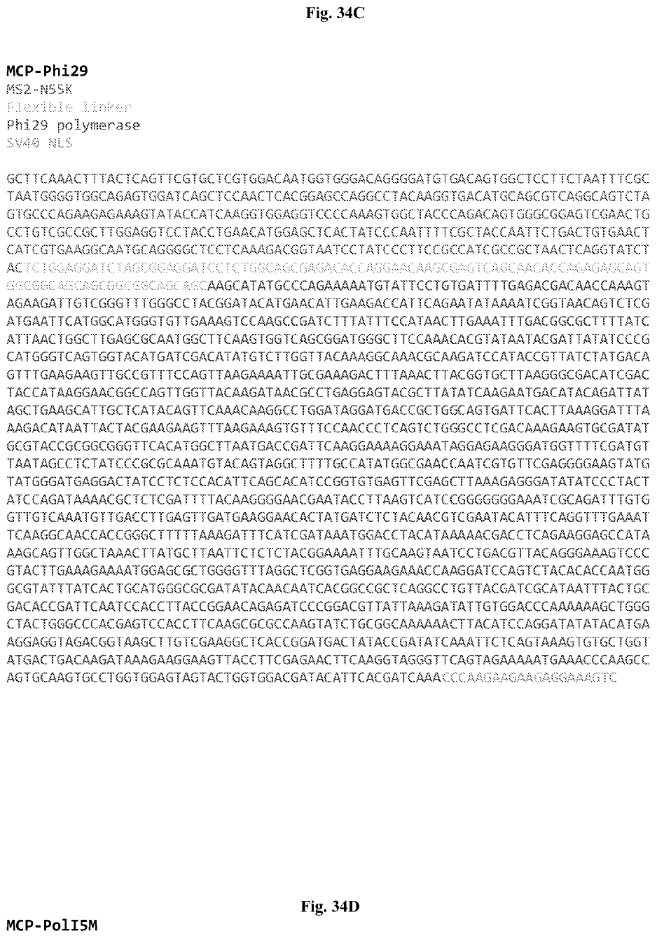

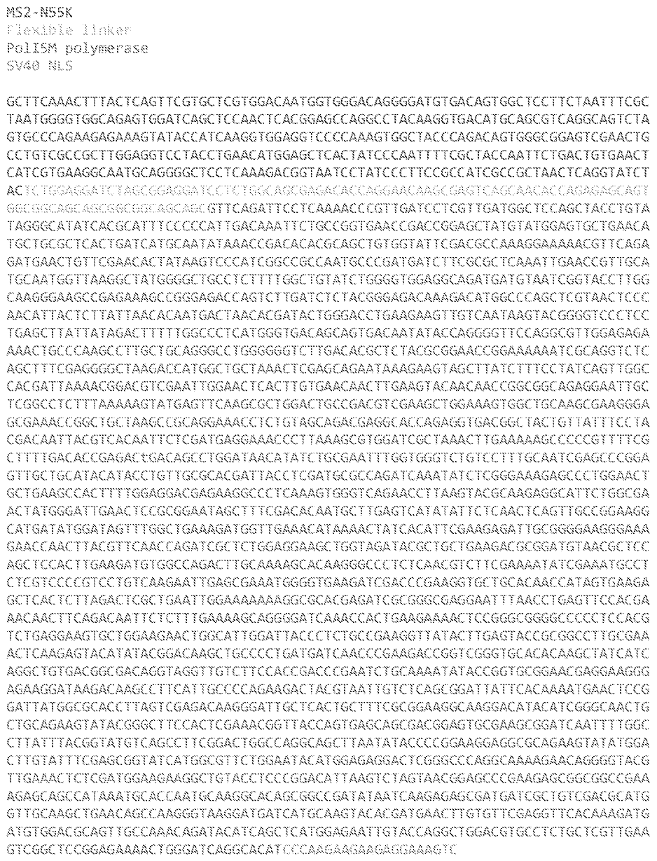

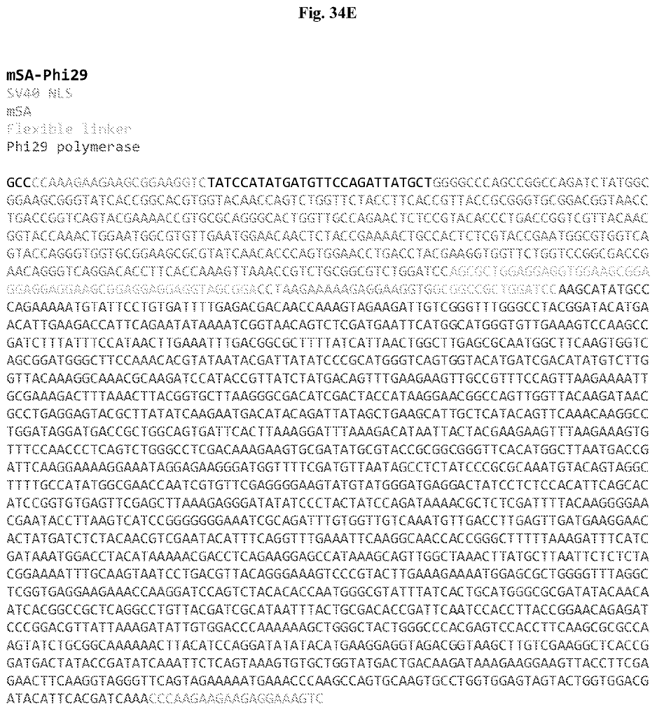

These linpet constructs are also capable of prime editing activity with long oligonucleotide insertion sequences. In particular, two AAVS sites (e.g., 1705 and 1615) were targeted with either a conventional MCP-RT and a chimeric MCP-HFPhi29Pol prime editor. At either AAVS site, the data show superior prime editing of a simultaneous 40 bp insertion/90 nt deletion with a chimeric MCP-HFPhi29Pol construct. See, FIGS. 33A and 33B. To perform long oligonucleotide prime editing, twin LPETs were used simultaneously. See, FIG. 33C. For example, a twin LPET construct resulted in the precise prime editing of a 132 nt insertion that also was superior with a chimeric MCP-HFPhi29Pol construct versus a conventional MCP-RT construct. See, FIG. 33D.

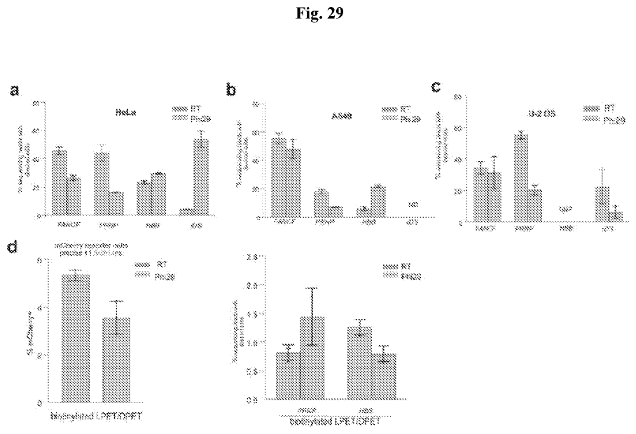

In vivo modular cPE-NTPol prime editing was performed in various cell lines (MDA-MB-231; U20S; HeLa and A549) on a FANCF gene comparing a cPE-RT(+2) construct with a cPE-Phi29(Ome) construct. See, FIGS. 29A-D.

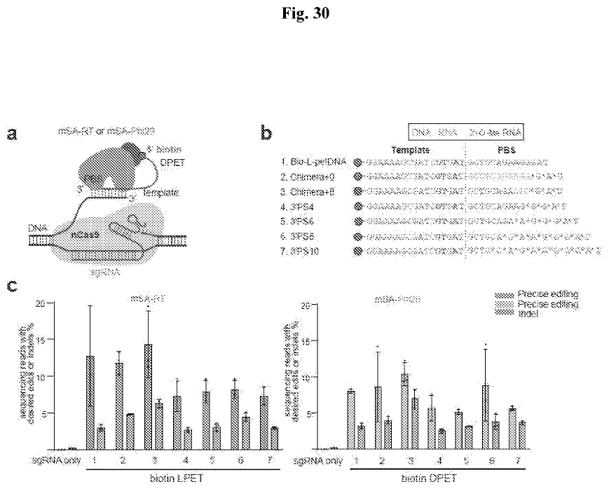

A modular cPE was constructed with a LPET or DPET appended with biotin (e.g., a bio-linpet) and a Cas9 fusion protein comprising either an RT protein or an HFPhi29Pol protein with streptavidin (e.g., mSA-RT or mSA-Phi29). See, FIG. 30A. The bio-LPET/DPET binds to an RT protein or a Phi29 protein via biotin-streptavidin. See, FIG. 30B. Successful prime editing with various bio-LPET/DPET constructs at a FANCF gene was observed when using either an mSA-RT or an mSA-Phi29. See, FIG. 30C.

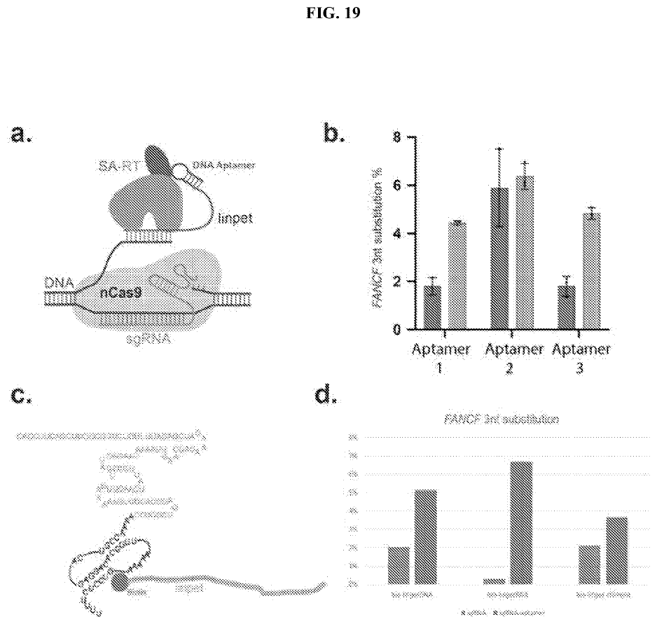

A modular cPE was also constructed with a linpet appended with a DNA aptamer that binds to an RT protein (e.g., SA-RT). See, FIG. 19A. Three different aptamers were successfully demonstrated for prime editing of the FANCF gene 3 nt insertion efficiency. See, FIG. 19B. A bio-linpet construct was used to compare prime editing with either an sgRNA or a sgRNA-aptamer. See FIG. 19C. The data show that the sgRNA-aptamer construct outperformed the conventional sgRNA construct whether the aptamer was DNA, RNA or a DNA/RNA chimera. See, FIG. 19D.

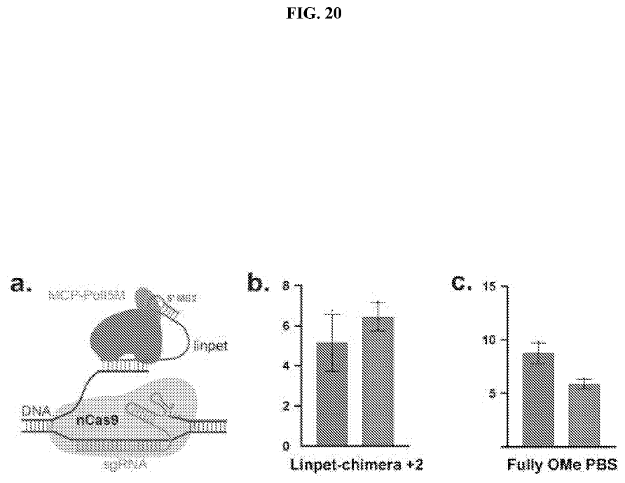

A modular cPE was constructed with a linpet appended with an MS2 aptamer that binds to a DNA polymerase protein (e.g., MCP-Pol15M). See, FIG. 20A. An MCP-Pol15M cPE in the presence of linpet-chimera (+2), either with or without a fully O-methylated primer binding site, demonstrated successful prime editing activity. See, FIGS. 20B and 20C, respectively.

### V. Processive, High Fidelity Polymerases

In one embodiment, the present invention contemplates a composition comprising a processive, high-fidelity, DNA or RNA-dependent DNA polymerase (HFPhi29Pol), untethered from Cas9, for gene editing in conjunction with a synthetic, end-stabilized, DNA or RNA-containing template. In one embodiment, the gene editing comprises a 60% efficiency of precise editing. Compared to conventional prime editing guide RNAs (pegRNAs), systems with LPETs and DPETs that are not fused to sgRNAs avoid autoinhibitory intramolecular base pairing interactions, improve the efficiency of chemical synthesis and modification, and support long templates (e.g., >100 nts) for larger insertions. Although it is not necessary to understand the mechanism of an invention, it is believed that the presently disclosed high fidelity polymerase system with exogenous templates has numerous advantages in the precision and versatility of genome editing, and in the production and delivery of the required components.

In one embodiment, high fidelity DNA polymerase includes, but is not limited to, at least one high fidelity characteristic. For example, such characteristics preferably include, but are not limited to an error rate of approximately 10⁻⁶ nucleotides, the ability to read a template that is greater than one hundred nucleotides in length with no errors and an affinity for deoxynucleotide triphosphates (dNTP) of approximately 1-100 nanomolar.

For example, a high fidelity DNA polymerase (e.g., Phi29) may comprise a dNTP affinity of approximately 10-30 nanomolar. In comparison, a conventional reverse transcriptase may be expected to have a dNTP affinity of approximately 10-100 micromolar. It is noteworthy that in the in vivo environment, most cells have generally low dNTP concentrations because they are either not cycling or not in the S phase if cycling is occurring. It is known that the affinity of MMLV RT for dNTPs is not great—reportedly 18-115 micromolar, depending on the dNTP. Oscorbin et al., Comp. Struct. Biotech J. 19:6315-6327 (2021). By contrast, the affinity of Phi29 DNA polymerase for dNTPs is approximately 1000-fold higher than that of MMLV-RT. See, Truniger et al., J. Mol. Biol. 335:481-449 (2004). Basically, this means that Phi29 would be expected to achieve more efficient flap synthesis than an RT with sub-micromolar intracellular dNTP concentrations. It is predictable that this difference in efficiency would only get bigger as the length of the desired edit increases.

Evidence suggests that the Phi29 DNA polymerase is a high fidelity DNA polymerase Estaban et al., “Fidelity of 029 DNA Polymerase: Comparison Between Protein-Primed Initiation and DNA Polymerization” J. Biol. Chem. 268(4):2719-2726 (1993). Phi29 DNA polymerase is reported to be a high fidelity proofreading enzyme with a very low replication error rate of 1×10⁻⁶-10⁻⁷ nucleotides both in its intrinsic enzymatic activity and during the amplification reaction. Other polymerases have been reported to have error rates similar to Phi29. See, Table I.

TABLE I
Reported Error Rates of High Fidelity Polymerases

| | Published | |
|---|---|---|
| | error rate | |
| | (errors/bp/ | |
| Enzyme | duplication) | References |
| T7 DNA | 15 × 10⁻⁶ | Mattilla et al., NAR.9: 4967-4973 |
| Polymerase | | (1991) |
| Klenow Fragment | 18 × 10⁻⁶ | Bebenek et al., J. Biol. Chem., 265, |
| (large) | | 13878-13887 (1990) |
| Klenow Fragment | 100 × 10⁻⁶ | Bebenek et al., J. Biol. Chem., 265, |
| (3′→5′ exo) | | 13878-13887 (1990) |
| T4 DNA | <10⁻⁶ | Kunkel et al.,, J. Biol. Chem., 259, |
| Polymerase | | 1539-1545 (1984) |
| Phi29 | 10⁻⁶-10⁻⁷ | Estaban et al., (supra) |

Prime editing provides opportunities for precise genome engineering, independent of homology-directed repair (HDR), by adding pegRNA and RT to CRISPR-associated genome editing systems. Anzalone, A. V. et al. Search-and-replace genome editing without double-strand breaks or donor DNA. Nature 576, 149-157 (2019); Lin, Q. et al. High-efficiency prime editing with optimized, paired pegRNAs in plants. Nat. Biotechnol. 39, 923-927 (2021); Zhuang, Y. et al. Increasing the efficiency and precision of prime editing with guide RNA pairs. Nat. Chem. Biol. 18, 29-37 (2022); Wang, J. et al. Efficient targeted insertion of large DNA fragments without DNA donors. Nat. Methods 19, 331-340 (2022); Choi, J. et al. Precise genomic deletions using paired prime editing. Nat. Biotechnol. 40, 218-226 (2022); Jiang, T., Zhang, X.-O., Weng, Z. & Xue, W. Deletion and replacement of long genomic sequences using prime editing. Nat. Biotechnol. 40, 227-234 (2022); and Chen, P. J. & Liu, D. R. Prime editing for precise and highly versatile genome manipulation. Nat. Rev. Genet. 24, 161-177 (2023). However, pegRNA complexity can hinder gene editing capabilities and results in several disadvantages. Liu, B. et al. A split prime editor with untethered reverse transcriptase and circular RNA template. Nat. Biotechnol. 40, 1388-1393 (2022); Nelson, J. W. et al. Engineered pegRNAs improve prime editing efficiency. Nat. Biotechnol. 40, 402-410 (2022).

PegRNAs, which harbor an RT template (RTT) and primer binding sequence (PBS) at the 3′ end of a single-guide RNA (sgRNA), are typically ˜130 nucleotides (nt), or longer, in length in order to generate point mutations or small indels. In vitro production of such long single-stranded pegRNA is expensive, time-consuming, and difficult to accomplish with high yield and purity. Furthermore, unlike the sgRNA (which is protected by close association with the Cas9 protein), nuclease-sensitive 3′ extensions of pegRNA are susceptible to degradation in cells. Liu, Y. et al. Enhancing prime editing by Csy4-mediated processing of pegRNA. Cell Res. 31, 1134-1136 (2021); Zhang, G. et al. Enhancement of prime editing via xrRNA motif-joined pegRNA. Nat. Commun. 13, 1856 (2022); Li, X. et al. Enhancing prime editing efficiency by modified pegRNA with RNA G-quadruplexes. J. Mol. Cell Biol. 14, (2022); and Feng, Y. et al. Enhancing Prime Editing Efficiency and Flexibility with Tethered and Split pegRNAs. Protein Cell (2022) doi:10.1093/procel/pwac014. In addition, the PBS is inevitably complementary to the spacer region of the pegRNA, which may cause intramolecular misfolding and attenuate effector loading and editing efficiency. Liu, Y. et al. Enhancing prime editing by Csy4-mediated processing of pegRNA. Cell Res. 31, 1134-1136 (2021).

Unlike many other DNA polymerases, reverse transcriptase (RT) enzymes are relatively error-prone due in part to their lack of proofreading activity, yielding a misincorporation event in as few as ˜2,000 nt (for MMLV RT, ˜11,000 nt). Although RT is used for prime editing through its RNA-templated DNA polymerase activity, this error frequency becomes an ever-increasing issue as PE insertions get longer, or as the number of edited cells (e.g., in an entire human organ) gets larger. Furthermore, RT processivity is often modest, perhaps contributing to the difficulties of efficiently installing large insertions.

In contrast to these RT enzyme limitations, some DNA-dependent DNA polymerases are exceptionally accurate and processive. Wu et al., How DNA polymerases catalyze replication and repair with contrasting fidelity. Nat. Rev. Chem. 1, 0068 (2017). For example, DNA-dependent DNA polymerases have been tethered to Cas9 and used to diversify local sequences using endogenous DNA as a template in bacteria and yeast. Halperin, S. O. et al. CRISPR-guided DNA polymerases enable diversification of all nucleotides in a tunable window. Nature 560, 248-252 (2018); and Tou et al., Targeted Diversification in the S. cerevisiae Genome with CRISPR-Guided DNA Polymerase I. ACS Synth. Biol. 9, 1911-1916 (2020). However, high-fidelity, processive, DNA-dependent DNA polymerases have not been reported in combination with exogenous templates and nickase Cas9 (nCas9) for precision genome editing in mammalian cells.

### A. DNA-Dependent Reverse Transcriptases

Split prime editing (sPE) systems have been previously reported, in which the nCas9 is untethered from a reverse transcriptase (RT) or the PBS/RTT is untethered from the sgRNA, or both. Liu, B. et al. A split prime editor with untethered reverse transcriptase and circular RNA template. Nat. Biotechnol. 40, 1388-1393 (2022); Feng, Y. et al. Enhancing Prime Editing Efficiency and Flexibility with Tethered and Split pegRNAs. Protein Cell (2022) doi:10.1093/procel/pwac014; and Grünewald, J. et al. Engineered CRISPR prime editors with compact, untethered reverse transcriptases. Nat. Biotechnol. (2022) doi:10.1038/s41587-022-01473-1. MMLV-RT is known to accept DNA as a template, in addition to its canonical RNA-templated DNA synthesis activity. Ohtsubo, Y., Sasaki, H., Nagata, Y. & Tsuda, M. Optimization of single strand DNA incorporation reaction by Moloney murine leukaemia virus reverse transcriptase. DNA Res. 25, 477-487 (2018).

When the PBS/RTT is separate from the sgRNA, it can be tethered to the RT. For example, such tethering can be accomplished by a fusion with an MS2 coat protein (MCP)] via an MS2 stem-loop. Furthermore, the PBS/RTT can be generated as a circular prime editing template RNA (petRNA), stabilizing it against exonuclease degradation and preventing intramolecular spacer/PBS pairing. In contrast, linear petRNAs produced by intracellular transcription are less efficient at supporting sPE, probably due to instability against exonucleases. However, functional, linear prime editing template (LPET) molecules can also be produced by chemical synthesis, providing a stable incorporation of internal 2′-deoxynucleotides, as well as more extensive modifications at the termini to protect against exonuclease digestion. See, FIG. 21A.

A series of LPETs harboring 2′-deoxynucleotides and an MS2 stem-loop at the 5′ end for tethering to MCP-RT and templating DNA synthesis for prime editing were engineered and synthesized. The LPETs also had three 2′-O-methyl/phosphorothioate linkages at each end to protect against exonuclease digestion. LPETs were tested via co-electroporation with nCas9 mRNA, RT-MCP mRNA, the sPE sgRNA, and the nicking sgRNA (PE3 format). The components were designed to yield a 3 nt substitution in the FANCF locus.

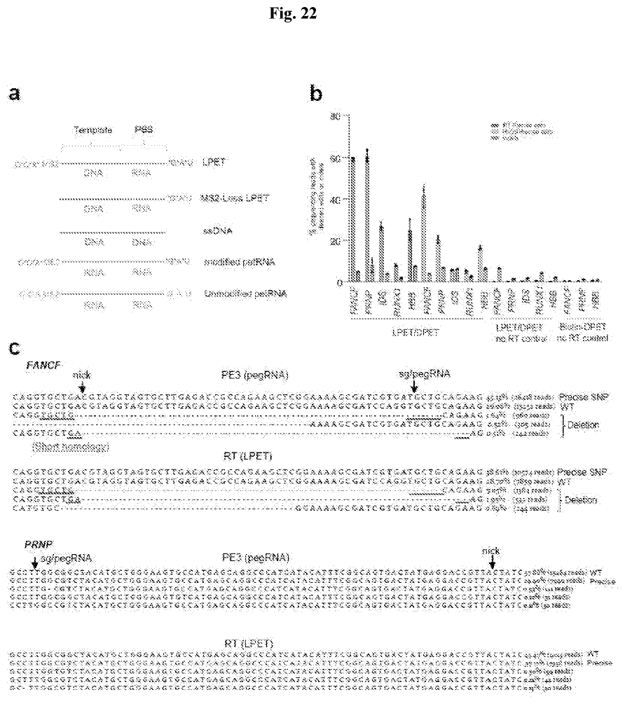

All RNA residues in the RTT were replaced with DNA and where efficient, dose-dependent prime editing from 10% to 50% was observed. See, FIG. 21B. This efficiency exceeded that observed previously (˜10%) with a linear petRNA expressed by intracellular transcription from a transfected plasmid8 as well as from the synthetic LPETs with or without chemical modifications (˜15%, ˜1.35%, respectively). See, FIG. 21C. No editing was detected when the MS2 stem-loop was removed as shown by data using MS2-less LPET and MS2-less ssDNA. See, FIG. 21C and FIG. 22A. These data suggest that the MS2-MCP tethering plays a role in gene editing. Furthermore, gene editing was absent in the “no RT” control groups at all tested sites, confirming that RT also plays a role in sPE. See, FIG. 22B. These results demonstrate that synthetic, partially DNA-containing LPETs support PE.

RT tolerance was tested using varying 2′-deoxyribose substitutions within the RTT and PBS regions. A fully 2′-deoxyribose-substituted RTT was synthesized (Chimera+0) as well as a full range of DNA/RN chimeric permutations including, but not limited to, the −17, −11, −6 and +11 positions. See, FIG. 21D. For example, the Chimera-17 template is an LPET lacking any 2′-deoxynucleotides, which is identical to the LPET with end modifications that was used in FIG. 21C. In contrast, the Chimera+11 LPET is fully DNA-substituted through the entire RTT and PBS. The LPETs with DNA replacements extending from −6 to +2 exhibit similarly efficient editing capabilities ranging from 45% to 60%. Further DNA replacements from +3 to +11 into the PBS region attenuated the editing efficiency. See, FIG. 21C. Although extensive PBS DNA substitution was detrimental, similarly extensive 2′-O-methylribose substitution in the PBS was well tolerated suggesting protection from nuclease activity. See, FIG. 21D.

LPET efficacy was also confirmed at two pathogenic sites, IDS (20%) and PRNP (50%) both of which were dependent on RT inclusion. See, FIG. 21E&F; and FIG. 22B. Small deletions were detected in both PE3 and LPET samples at FANCF, but not at PRNP. See, FIG. 22C. Consistent with previous PE studies, the deletions are likely caused by short homology at the FANCF nicking sites. Liu, P. et al. Improved prime editors enable pathogenic allele correction and cancer modelling in adult mice. Nat. Commun. 12, 2121 (2021).

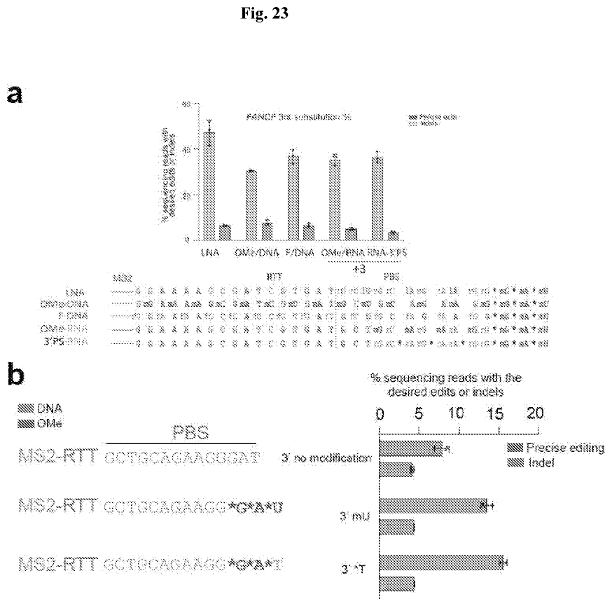

Other chemical modifications were also tested at various positions within the LPET, including, but not limited to, locked nucleic acid (LNA), 2′-O-methyl, phosphorothioate, 2′-fluoro, and/or combinations thereof. These configurations did not further improve gene editing efficiency, although the LPETs retained gene editing capabilities with these modifications. See, FIG. 23A. In addition, different end-protecting modifications were tested for an otherwise all-DNA RTT and PBS. Complete DNA replacement of the RTT and PBS RNA regions, with no 3′ end modification, achieved a 7% gene editing efficiency but this was increased ˜2-fold with a 3′ end modification. See, FIG. 23B. Together, these data suggest that DNA replacement in the RTT region is compatible with efficient prime editing.

### B. High Fidelity DNA/RNA-Dependent DNA Polymerase

As discussed above, DNA-containing templates can undergo gene editing with conventional efficiency with RT. In one embodiment, the present invention contemplates a cPE system comprising a DNA-dependent DNA polymerase configured to perform precision genome editing. This system would be an improved version of the EvolvR system. Halperin, S. O. et al. CRISPR-guided DNA polymerases enable diversification of all nucleotides in a tunable window. Nature 560, 248-252 (2018); and Tou et al. Targeted Diversification in the S. cerevisiae Genome with CRISPR-Guided DNA Polymerase I. ACS Synth. Biol. 9, 1911-1916 (2020). Such improvements include, but are not limited to, an nCas9 and DNA-dependent DNA polymerase untethered from each other, and/or with an exogenous template to support precision editing rather than sequence diversification. For example, a replicative DNA polymerase from Bacillus subtilis phage Phi29, which exhibits characteristics including, but not limited to, strand-displacement capability, robust processivity, high dNTP affinity, and high fidelity that are contained within a single ˜67 kDa polypeptide. See, FIG. 24A; Esteban et al., Fidelity of phi 29 DNA polymerase. Comparison between protein-primed initiation and DNA polymerization. J. Biol. Chem. 268, 2719-2726 (1993); and Lieberman, K. R. et al. Processive Replication of Single DNA Molecules in a Nanopore Catalyzed by phi29 DNA Polymerase. J. Am. Chem. Soc. 132, 17961-17972 (2010).

Chimeric LPETs as disclosed above were co-delivered with nCas9 and Phi29 as mRNAs. See, FIG. 21C. It was observed that HFPhi29Pol results in efficient precision editing of up to 50% with the LPET (chimera+2). An increased RNA content between the 0 to −6 position of the LPET design attenuates HFPhi29Pol editing activity. A fully DNA-containing template (chimera+0) results in a 30% gene editing efficiency, which can be further enhanced to 50% by increasing the DNA replacement to +2 bp within the PBS region. Additional DNA replacement within positions +5 to +11 of the PBS region reduces editing efficiency. See, FIG. 24B.

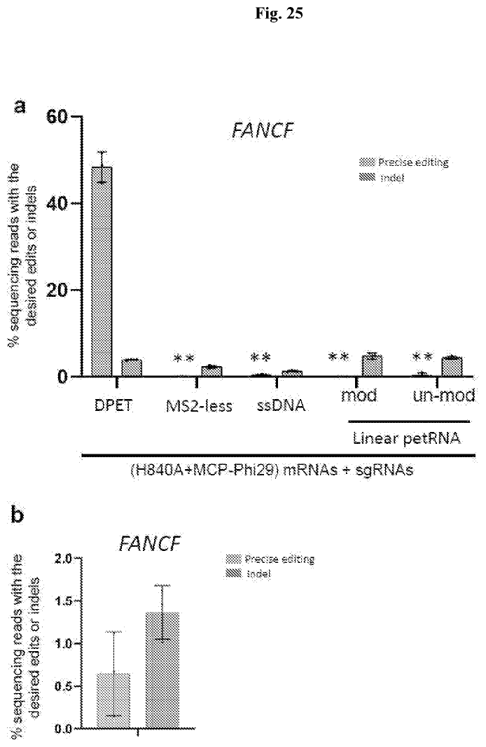

Similar to RT-based prime editing, Phi29-based editing was undetectable in the absence of the MS2 sequence on the LPET. See, FIG. 25A. Gene editing was undetectable with either an unmodified LPET RNA or a full-length pegRNA. See, FIGS. 25A and 25B, respectively.

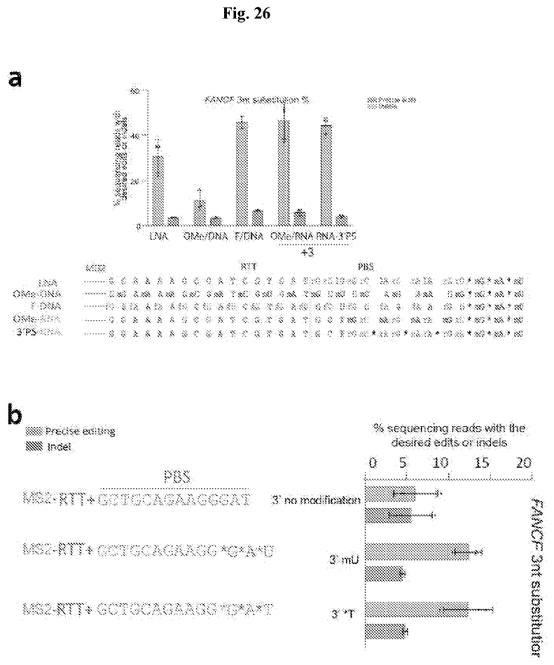

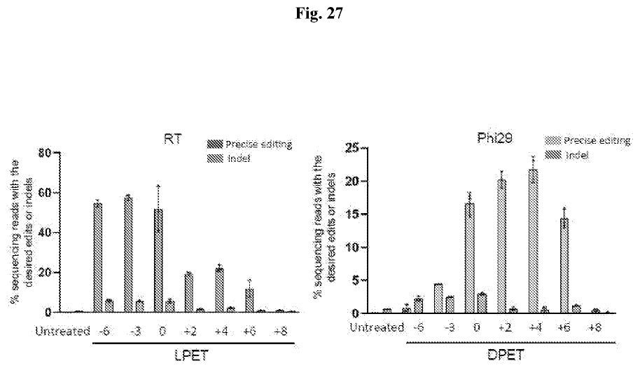

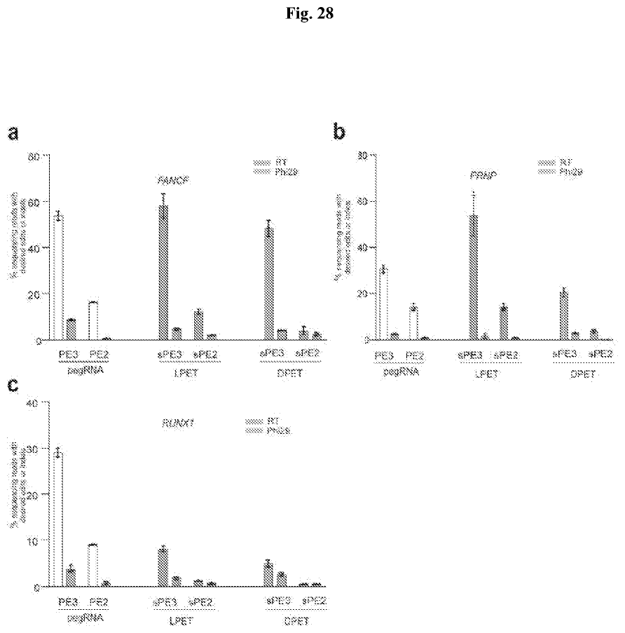

Full modification of the PBS with 2′-O-methyl residues further increased editing efficiency by 1.5 to 2-fold. See, FIGS. 24B and 24C. In contrast, DPET chemical modifications did not improve HFPhi29Pol gene editing efficiency. See, FIG. 26B. As the DNA substitution in the PBS/template regions of the PRNP DPET was increased and the editing pattern was similar to that of FANCF. See, FIG. 27; cf. FIG. 21D and FIG. 24B. In addition, canonical full-length pegRNAs and DPETs were tested at five different endogenous sites with or without nicking sgRNA using mRNA electroporation. This template also supported sPE editing (e.g., sPE2) while adding nicking sgRNA (e.g., sPE3) further increased editing. See, FIGS. 28A-C.

LPETs and DPETs were tested using sPE and DPE (respectively) in four commonly used cell lines (HeLa, A549, U20S, and MDA-MB-231). The synthetic templates resulted in efficient genome editing ranging between 20%-60% with RT or HFPhi29Pol in all four lines. See FIG. 24D; and FIGS. 29A-C.

LPETs and DPETs also resulted in precise genome editing in IMR90 primary human lung fibroblasts in two disease-related genes (PRNP, prion disease; IDS, Hunter Syndrome). To demonstrate protein-level readout of genome editing, a template was designed which repaired a silent mCherry reporter with a 4 nt insertion. Liu, B. et al., A split prime editor with untethered reverse transcriptase and circular RNA template. Nat. Biotechnol. 40, 1388-1393 (2022). Electroporation of nCas9, RT or Phi29, sgRNAs and this template restored mCherry expression in reporter cells. See, FIG. 24E. Together, these data indicate that the HFPhi29Pol DNA-dependent DNA polymerase supports efficient genome editing using DPETs in multiple cell lines, including primary human cells.

Because solid-phase synthesis of long RNA nucleotide templates have challenges including, but not limited to, deprotection, purification, cost, and stability it was advantageous to replace the MS2 stem-loop RNA as an affinity module to non-covalently tether the PBS and template to the polymerase with a molecule that would obviate the need for RNA residues in the LPET or DPET. To this end, a monomeric streptavidin (mSA) was fused to an N-terminus of either RT or HFPhi29Pol. See, FIG. 30. To tether the templates to the respective polymerases, a series of 5′-biotinylated templates were synthesized with different additional modification patterns.

The data showed that an mSA/biotin tethering system results in sPE (e.g., RT) or DPE (e.g., HFPhi29Pol) at multiple endogenous sites and with the mCherry reporter. See, FIG. 30; and FIG. 29. Gene editing efficiencies ranged between 1-20% for mSA-RT and 1-14% for mSA-HFPhi29Pol which are reduced relative to those observed with MS2/MCP tethering.

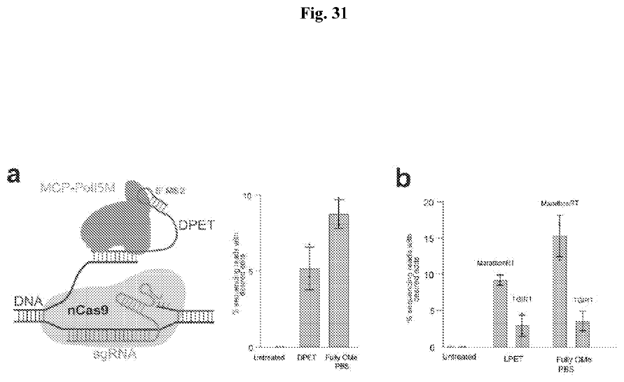

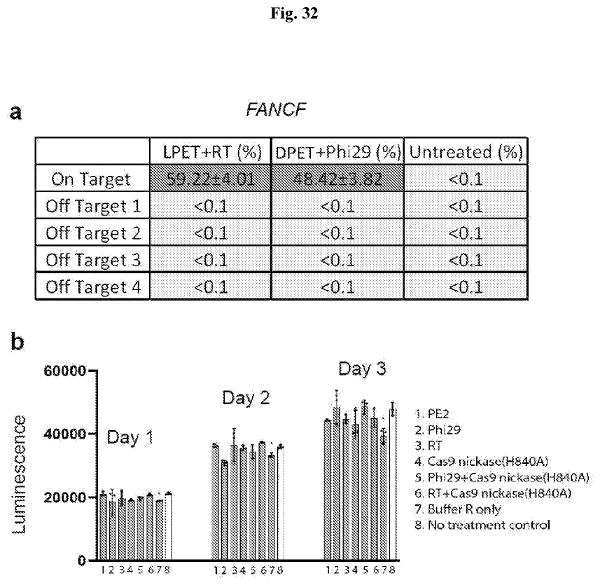

A high fidelity DNA polymerases PoI15M, a variant of PolI3M23 was then tested in the DPET system. In particular, DPETs with a +2 modification pattern, or with a fully 2′-O-methyl-substituted PBS were used. PolI5M resulted in a gene editing efficiency of between 5%-10%. See, FIG. 31. An MMLV-RT protein was also replaced with two bacterial RTs, MarathonRT and TGIRT. MarathonRT resulted between ˜10%-15% editing when using either a (+2) or O-me LPET, whereas TGIRT efficiency was <5%. See, FIG. 31. Together, these data suggested LPETs and DPETs have considerable flexibility for performing either sPE or DPE with multiple polymerase modules. In addition, both the LPET/RT and DPET/HFPhi29Pol configurations exhibited only background levels of off-target editing at known FANCF SpyCas9 off-target sites Furthermore, neither RT nor HFPhi29Pol induced detectable toxicity relative to that of Cas9 nickase alone. See, FIG. 32.

Another disadvantage of solid-phase full-length pegRNA synthesis that require extended template sequences is the generation of insertions including, but not limited to recombinase site insertions (e.g., AttB/AttP or loxP), epitope tags, and other desirable edits. The separate and independent nature of the presently disclosed LPET and DPET systems solve this problem by: i) physically separating the sgRNA and PBS/template into two independent molecules; and ii) providing 2′-deoxy, 2′-O-methyl nucleotide modifications and other synthetically favorable residues.

To test whether an sPE or DPE synthetic template system is compatible with recombination site insertions, a single template was used to insert a 40 nt loxP site into an AAVS locus. A 40% and 35% insertion efficiency was observed with either sPE or DPE, respectively. See, FIGS. 33A and 33B. (Extended Data FIG. 10a, b).

The TwinPE system, and its DPE analog, were tested with the presently disclosed synthetic templates to support even longer insertions. To this end, paired, overlapping, templates were tested for insertion of a 132 nt sequence into the HEK3 site. Droplet digital PCR (ddPCR) measurements revealed insertion efficiencies of 6% and 8% by TwinPE/RT and TwinDPE/HFPhi29Pol, respectively. See, FIGS. 33C and 33D. Together, these data suggest that synthetic modular templates support insertion of extended templates.

### C. Practical Applications

In one embodiment, the present invention contemplates a DNA-dependent DNA polymerase, untethered from nCas9, to perform DNA polymerase editing (DPE) with synthetic, modified, linear DPE templates (DPETs) for precise genome editing in mammalian cells. The use of DNA templates with modified nucleotides instead of conventional RNA templates provides numerous advantages regarding synthesis length, efficiency, throughput, cost, scale, nuclease resistance, and versatility. As with previously reported circular petRNAs and sPE, synthetic DPETs (and LPETs) avoid misfolding errors due to the autoinhibitory-intramolecular PBS/spacer complementarity that is intrinsic to pegRNAs. Furthermore, DPET and LPET chemical modifications can prevent polymerase read-through beyond the intended template region, which is another disadvantage when using conventional pegRNAs. Finally, the use of HFPhi29Pol, a DNA-dependent DNA polymerase with exceptional fidelity and processivity, allows for precise genome editing by improved editing accuracy and efficiency with large insertion sequence lengths (e.g., >100 nts). As PE and DPE strategies advance toward longer insertions, or toward larger numbers of edited cells (such as an entire human organ in vivo), high fidelity polymerase systems for precision editing that are highly accurate and processive (e.g., HFPhi29Pol) can successfully meet the need.

In one embodiment, the present invention contemplates a nanoparticle delivery system comprising a nicking Cas9 protein, an mRNA encoding either an RT or an HFPhi29Pol and an sgRNA encoding an LPET or a DPET. In one embodiment, the delivery system is non-viral. In one embodiment, the non-viral delivery system is a liposomal nucleoprotein (LNP) delivery system. Although it is not necessary to understand the mechanism of an invention, it is believed that an LNP delivery system overcomes disadvantages of conventional PE effector sizes (e.g., PE2 >6.3 kb).

It has been reported that the synthesis of pegRNAs with long RTT sequences, such as twin-pegRNA and GRAND have several challenges and are therefore disadvantageous. Wang, J. et al. Efficient targeted insertion of large DNA fragments without DNA donors. Nat. Methods 19, 331-340 (2022); Nelson, J. W. et al. Engineered pegRNAs improve prime editing efficiency. Nat. Biotechnol. 40, 402-410 (2022); and Anzalone, A. V. et al. Programmable deletion, replacement, integration and inversion of large DNA sequences with twin prime editing. Nat. Biotechnol. (2021) doi:10.1038/s41587-021-01133-w. The presently disclosed DNA-containing LPETs and DPETs solve these problems with MS2 stem-loops are ˜60 nts as compared to pegRNAs, which are minimally ˜125-130 nts. The use of other tethering modules such as biotin also reduces LPET/DPET length requirements as well as reducing the number of ribonucleotides needed for tethering. Generating templates separately from the cognate sgRNAs ameliorates the synthetic length and yield limitations of pegRNAs and offers production advantages. As editing efficiencies increase via optimization of tethering systems and effector/template configurations, the high fidelity and processivity inherent to Phi29-based DPE are advantageous for genome editing in vivo via non-viral delivery. Gillmore, J. D. et al. CRISPR-Cas9 In Vivo Gene Editing for Transthyretin Amyloidosis. N. Engl. J. Med. 385, 493-502 (2021); and Lee, R. G. et al. Efficacy and Safety of an Investigational Single-Course CRISPR Base-Editing Therapy Targeting PCSK9 in Nonhuman Primate and Mouse Models. Circulation 147, 242-253 (2023).

Although RT-based prime editing supports efficient editing in human cells, the art lacks a comprehensive understanding of potential byproducts that might be unique to RT activity templated by abundant cellular RNAs. This could include self-primed cDNAs that may be recombinogenic. For example, residual or transiently cytoplasmic RT could produce cytoplasmic cDNA with the potential to prompt innate immune responses by the cGAS/STING pathway. Hopfner et al., Molecular mechanisms and cellular functions of cGAS-STING signaling. Nat. Rev. Mol. Cell Biol. 21, 501-521 (2020). In contrast, DNA-dependent DNA polymerases support housekeeping functions such as DNA repair and (in cycling cells) genome replication. It is therefore likely that even unanticipated activities of exogenous, high-fidelity, DNA-dependent DNA polymerases would be well tolerated. HFPhi29Pol is exemplified herein because of its advantageous properties as a single polypeptide, but it is expected that screening for the high fidelity characteristics disclosed herein in other polymerases will have similar advantages for genome editing. Overall, the flexibility of PE polymerase modules and template chemistries as disclosed herein result many new and distinct applications.

### V. Adeno-Associated Viruses

Adeno-associated viruses (AAV) are small viruses that infect humans and some other primate species. AAVs are small (20 nm) replication-defective, nonenveloped viruses and have linear single-stranded DNA (ssDNA) genome of approximately 4.8 kilobases (kb). Naso et al., “Adeno-Associated Virus (AAV) as a Vector for Gene Therapy” BioDrugs 31(4):317-334 (2017); and Wu et al., “Effect of Genome Size on AAV Vector Packaging” Molecular Therapy 18 (1): 80-86 (2010). AAVs are not currently known to cause disease. The viruses cause a very mild immune response. Several additional features make AAV an attractive candidate for creating viral vectors for gene therapy, and for the creation of isogenic human disease models. Grieger et al., “Adeno-associated Virus as a Gene Therapy Vector: Vector Development, Production and Clinical Applications”; Adeno-associated virus as a gene therapy vector: vector development, production and clinical applications. In: Advances in Biochemical Engineering/Biotechnology. 99. pp. 119-145 (2005). Gene therapy vectors using AAV can infect both dividing and quiescent cells and persist in an extrachromosomal state without integrating into the genome of the host cell, although in the native virus integration of virally carried genes into the host genome does occur. Deyle et al., “Adeno-associated virus vector integration”. Current Opinion in Molecular Therapeutics. 11(4):442-447 (2009).

Development of AAVs as gene therapy vectors eliminated the genomic integration capacity by removal of the rep and cap genes. The modified vector has a promoter to drive transcription of the carried gene which is inserted between inverted terminal repeats (ITRs). AAV-based gene therapy vectors consequently form episomal concatemers in the host cell nucleus. In non-dividing cells, these concatemers remain intact for the life of the host cell. In dividing cells, AAV DNA is lost through cell division, since the episomal DNA is not replicated along with the host cell DNA. Surosky et al., “Adeno-associated virus Rep proteins target DNA sequences to a unique locus in the human genome” Journal of Virology 71(10):7951-7959 (1997).

The AAV genome is built of single-stranded deoxyribonucleic acid (ssDNA), either positive- or negative-sensed, which is about 4.7 kilobase long. The genome comprises ITRs at both ends of the DNA strand, and two open reading frames (ORFs) encoding the rep and cap proteins. The rep ORF is composed of four overlapping genes encoding Rep proteins required for the AAV life cycle. The cap ORF is composed of overlapping nucleotide sequences of capsid proteins (e.g., VP1, VP2 and VP3) which interact to form a capsid with icosahedral symmetry. Carter B J, “Adeno-associated virus and adeno-associated virus vectors for gene delivery”. In: Lassic D D, Templeton N S (eds.). Gene Therapy: Therapeutic Mechanisms and Strategies. New York City: Marcel Dekker, Inc. pp. 41-59 (2000).

AAV inverted terminal repeat (ITR) sequences usually comprise about 145 bases each and are believed required for efficient multiplication of the AAV genome. Bohenzky et al., “Sequence and symmetry requirements within the internal palindromic sequences of the adeno-associated virus terminal repeat” Virology 166(2):316-327 (1988). ITRs also have a hairpin structure which contributes to self-priming that allows a primase-independent synthesis of the second DNA strand. The ITRs were also shown to be required for host cell DNA integration/removal, efficient encapsidation and deoxyribonuclease resistance. Wang et al., “Rescue and replication signals of the adeno-associated virus 2 genome” Journal of Molecular Biology 250(5):573-580 (1995); Weitzman et al., “Adeno-associated virus (AAV) Rep proteins mediate complex formation between AAV DNA and its integration site in human DNA” PNAS USA 91(13): 5808-5812 (1994); and Zhou et al, “In vitro packaging of adeno-associated virus DNA”. Journal of Virology 72(4):3241-3247 (1998). With regard to gene therapy, ITRs are configured in cis next to the therapeutic gene, in contrast the structural (cap) and packaging (rep) proteins which can be delivered in trans. Nony et al., “Novel cis-acting replication element in the adeno-associated virus type 2 genome is involved in amplification of integrated rep-cap sequences” Journal of Virology 75(20):9991-9994 (2001); Nony et al., “Evidence for packaging of rep-cap sequences into adeno-associated virus (AAV) type 2 capsids in the absence of inverted terminal repeats: a model for generation of rep-positive AAV particles” Journal of Virology 77(1):776-781 (2003); Philpott et al., “Efficient integration of recombinant adeno-associated virus DNA vectors requires a p5-rep sequence in cis” Journal of Virology 76(11):5411-5421 (June 2002); and Tullis et al., “Efficient replication of adeno-associated virus type 2 vectors: a cis-acting element outside of the terminal repeats and a minimal size”. Journal of Virology 74(24):11511-11521 (2000).

### VI. cPE Treatment Of Genetic Disease

In one embodiment, the present invention contemplates a method for treating a genetic disease with a composition comprising a cPE. In one embodiment the cPE is a modular cPE. For example, administration of a modular cPE to a patient comprising a genetic mutation loci, reverts the mutated loci into a wild type loci through a nucleotide transversion (e.g., 1 nt, 2 nt, 3 nt etc., transversion). Such mutated loci may be located on genes including, but not limited to, an HBB gene, a HEXA gene, a PSEN1 gene, a PRNP gene, an IDS gene and/or an IDUA gene.

### A. Hemaglobin Subunit B (HBB) Genetic Diseases

The HBB gene encodes a protein called beta-globin. Beta-globin is a component (subunit) of a larger protein called hemoglobin, which is located inside red blood cells. In adults, hemoglobin normally consists of four protein subunits: two subunits of beta-globin and two subunits of a protein called alpha-globin, which is produced from another gene called HBA. Each of these protein subunits is attached (bound) to an iron-containing molecule called heme; each heme contains an iron molecule in its center that can bind to one oxygen molecule. Hemoglobin within red blood cells binds to oxygen molecules in the lungs. These cells then travel through the bloodstream and deliver oxygen to tissues throughout the body.

### 1. Beta Thalassemia

Nearly 400 mutations in the HBB gene have been found to cause beta thalassemia. Most of the mutations involve a change in a single DNA nucleotide within or near the HBB gene. Other mutations comprise an insertion or deletion of nucleotides in the HBB gene.

HBB gene mutations that decrease beta-globin production result in a condition called beta-plus (β+) thalassemia. Mutations that prevent cells from producing any beta-globin result in beta-zero (β0) thalassemia.

Problems with the subunits that make up hemoglobin, including low levels of beta-globin, reduce or eliminate the production of this molecule. A lack of hemoglobin disrupts the normal development of red blood cells. A shortage of mature red blood cells can reduce the amount of oxygen that is delivered to tissues to below what is needed to satisfy the body's energy needs. A lack of oxygen in the body's tissues can lead to poor growth, organ damage, and other health problems associated with beta thalassemia.

### 2. Methemoglobinemia, Beta-Globin Type

More than 10 mutations in the HBB gene have been found to cause methemoglobinemia, beta-globin type, which is a condition that alters the hemoglobin within red blood cells. These mutations often affect the region of the protein that binds to heme. For hemoglobin to bind to oxygen, the iron within the heme molecule needs to be in a form called ferrous iron (Fe²⁺). The iron within the heme can change to another form of iron called ferric iron (Fe³⁺), which cannot bind to oxygen. Hemoglobin that contains ferric iron is known as methemoglobin and is unable to efficiently deliver oxygen to the body's tissues.

In methemoglobinemia, beta-globin type, mutations in the HBB gene alter the beta-globin protein and promote the heme iron to change from ferrous to ferric. This altered hemoglobin gives the blood a brown color and causes a bluish appearance of the skin, lips, and nails (cyanosis). The signs and symptoms of methemoglobinemia, beta-globin type are generally limited to cyanosis, which does not cause any health problems. However, in rare cases, severe methemoglobinemia, beta-globin type can cause headaches, weakness, and fatigue.

### 3. Sickle Cell Disease

Sickle cell anemia (also called homozygous sickle cell disease or HbSS disease) is the most common form of sickle cell disease. This form is caused by a particular mutation in the HBB gene that results in the production of an abnormal version of beta-globin called hemoglobin S or HbS. In this condition, hemoglobin S replaces both beta-globin subunits in hemoglobin. The mutation that causes hemoglobin S changes a single protein building block (amino acid) in beta-globin. Specifically, the amino acid glutamic acid is replaced with the amino acid valine at position 6 in beta-globin (e.g., Glu6Val or E6V). Replacing glutamic acid with valine causes the abnormal hemoglobin S subunits to stick together and form long, rigid molecules that bend red blood cells into a sickle (crescent) shape. The sickle-shaped cells die prematurely, which can lead to a shortage of red blood cells (anemia). The sickle-shaped cells are rigid and can block small blood vessels, causing severe pain and organ damage.

Mutations in the HBB gene can also cause other abnormalities in beta-globin, leading to other types of sickle cell disease. These abnormal forms of beta-globin are often designated by letters of the alphabet or sometimes by a name. In these other types of sickle cell disease, just one beta-globin subunit is replaced with hemoglobin S. The other beta-globin subunit is replaced with a different abnormal variant, such as hemoglobin C or hemoglobin E.

In hemoglobin SC (HbSC) disease, the beta-globin subunits are replaced by hemoglobin S and hemoglobin C. Hemoglobin C results when the amino acid lysine replaces the amino acid glutamic acid at position 6 in beta-globin (e.g., Glu6Lys or E6K). The severity of hemoglobin SC disease is variable, but it can be as severe as sickle cell anemia. Hemoglobin E (HbE) is caused when the amino acid glutamic acid is replaced with the amino acid lysine at position 26 in beta-globin (written Glu26Lys or E26K). In some cases, the hemoglobin E mutation is present with hemoglobin S. In these cases, a person may have more severe signs and symptoms associated with sickle cell anemia, such as episodes of pain, anemia, and abnormal spleen function.

Other conditions, known as hemoglobin sickle-beta thalassemias (HbSBetaThal), are caused when mutations that produce hemoglobin S and beta thalassemia occur together. Mutations that combine sickle cell disease with beta-zero (β0) thalassemia lead to severe disease, while sickle cell disease combined with beta-plus (β+) thalassemia is generally milder.

### 4. Other HBB Diseases

Hundreds of variations have been identified in the HBB gene. These changes result in the production of different versions of beta-globin. Some of these variations cause no noticeable signs or symptoms and are found when blood work is done for other reasons, while other HBB gene variations may affect a person's health. Two of the most common variants are hemoglobin C and hemoglobin E.

Hemoglobin C (HbC), caused by the Glu6Lys mutation in beta-globin, is more common in people of West African descent than in other populations. People who have two hemoglobin C subunits in their hemoglobin, instead of normal beta-globin, have a mild condition called hemoglobin C disease. This condition often causes chronic anemia, in which the red blood cells are broken down prematurely.

Hemoglobin E (HbE), caused by the Glu26Lys mutation in beta-globin, is a variant of hemoglobin most commonly found in the Southeast Asian population. When a person has two hemoglobin E subunits in their hemoglobin in place of beta-globin, a mild anemia called hemoglobin E disease can occur. In some cases, the mutations that produce hemoglobin E and beta thalassemia are found together. People with this hemoglobin combination can have signs and symptoms ranging from mild anemia to severe thalassemia major.

### B. Hexosaminidase, Subunit Alpha (HEXA) Genetic Diseases

The HEXA gene encodes a subunit of an enzyme called beta-hexosaminidase A. Specifically, a protein produced from a HEXA gene forms the alpha subunit of this enzyme. One alpha subunit joins with one beta subunit (produced from the HEXB gene) to form a functioning beta-hexosaminidase A enzyme.

Beta-hexosaminidase A is believed to play a role in brain and spinal cord function. Beta-hexosaminidase A is generally found in lysosomes, which are structures in cells that break down toxic substances and act as recycling centers. Within lysosomes, beta-hexosaminidase A forms part of a complex that breaks down a fatty substance called GM2 ganglioside found in cell membranes.

### 1. Tay-Sachs Disease

More than 210 mutations that cause Tay-Sachs disease have been identified in the HEXA gene. Tay-Sachs disease is a condition characterized by movement disorders, intellectual and developmental disability, and other neurological problems caused by the death of nerve cells in the central nervous system.

The HEXA gene variants that cause Tay-Sachs disease eliminate or severely reduce the activity of the enzyme beta-hexosaminidase A. This lack of enzyme activity prevents the enzyme from breaking down GM2 ganglioside. As a result, this substance builds up to toxic levels, particularly in neurons in the central nervous system. Progressive damage caused by the buildup of GM2 ganglioside leads to the destruction of these cells, which causes the signs and symptoms of Tay-Sachs disease.

Most of the known HEXA gene variants result in a completely nonfunctional version of beta-hexosaminidase A. These variants cause a severe form of Tay-Sachs disease, known as infantile Tay-Sachs disease, which appears in infancy. Other variants severely reduce but do not eliminate the activity of beta-hexosaminidase A. These genetic changes are responsible for less severe forms of Tay-Sachs disease, known as the juvenile and late-onset forms, which appear later in life.

### C. Presenilin 1 (PSEN1) Genetic Diseases

The PSEN1 gene encodes a protein called presenilin 1. This protein is a subunit of a gamma- (γ-) secretase complex. Presenilin 1 has proteolytic activity and cleaves other proteins into peptides.

The γ-secretase complex is located in the cell membrane where it cleaves many different proteins types of transmembrane proteins. This cleavage process participates in transmembrane signaling pathways that transmit biochemical messages from outside the cell into the nucleus. One of these pathways, known as Notch signaling, plays a role in the normal maturation and division of hair follicle cells and other types of skin cells. Notch signaling is also involved in normal immune system function.

The γ-secretase complex may be best known for its role in processing amyloid precursor protein (APP), which is made in the brain and other tissues. γ-secretase cuts APP into smaller peptides, including soluble amyloid precursor protein (sAPP) and several versions of amyloid-beta (β) peptide. Evidence suggests that sAPP has growth-promoting properties and may play a role in the formation of nerve cells (neurons) in the brain both before and after birth. Other functions of sAPP and amyloid-β peptide are under investigation.

### 1. Alzheimer Disease

More than 150 PSEN1 gene mutations have been identified in patients with early-onset Alzheimer disease, a degenerative brain condition that begins before age 65. Mutations in the PSEN1 gene are the most common cause of early-onset Alzheimer disease, accounting for up to 70 percent of cases.

Almost all PSEN1 gene mutations change single DNA nucleotides in a particular segment of the PSEN1 gene. These mutations result in the production of an abnormal presenilin 1 protein. Defective presenilin 1 interferes with the function of the γ-secretase complex, which alters the processing of APP and leads to the overproduction of a longer, toxic version of amyloid-β peptide. Copies of this protein fragment stick together and build up in the brain, forming clumps called amyloid plaques that are a characteristic feature of Alzheimer disease. A buildup of toxic amyloid-β peptide and the formation of amyloid plaques likely lead to the death of neurons and the progressive signs and symptoms of this disorder.

### 2. Hidradenitis Suppurativa

At least one mutation in the PSEN1 gene has been found to cause hidradenitis suppurativa, a chronic skin disease characterized by recurrent boil-like lumps (nodules) under the skin that develop in hair follicles. The nodules tend to become inflamed and painful, and they produce significant scarring as they heal.

The identified mutation deletes a single DNA nucleotide from the PSEN1 gene (e.g., 725delC). This genetic change reduces the amount of functional presenilin 1 produced in cells, so less of this protein is available to act as part of the γ-secretase complex. The resulting shortage of normal γ-secretase impairs cell signaling pathways, including Notch signaling. Although little is known about the mechanism, studies suggest that abnormal Notch signaling may promote the development of recurrent nodules in hair follicles and trigger inflammation in the skin.

Studies suggest that the PSEN1 gene mutation that causes hidradenitis suppurativa has a different effect on γ-secretase function than the mutations that cause early-onset Alzheimer disease. These differences may explain why no single PSEN1 gene mutation has been reported to cause the signs and symptoms of both diseases.

### D. Prion Protein (PRNP) Genetic Diseases

The PRNP gene encodes a protein called prion protein (PrP), which is active in the brain and several other tissues. Although the precise function of this protein is unknown, researchers have proposed roles in several processes. These include, but are not limited to, the transport of copper into cells and/or protection of brain cells (neurons) from injury (neuroprotection). Studies have also suggested a role for PrP in the formation of synapses, which are the junctions between nerve cells (neurons) where cell-to-cell communication occurs.

Different forms of PrP have been identified. The normal version is often designated PrP^{C} to distinguish it from abnormal forms of the protein, which are generally designated PrP^{Sc}.

### 1. Huntington Disease-Like Syndrome

A particular type of mutation in the PRNP gene has been found to cause signs and symptoms that resemble those of Huntington disease, including uncontrolled movements, emotional problems, and loss of thinking ability. Researchers have proposed that this condition be called Huntington disease-like 1 (HDL 1).

The PRNP mutations associated with HDL1 are believed located in an octapeptide repeat. This repeat encodes eight (8) amino acids and is normally repeated five times in the PRNP gene. In people with HDL1, this segment is repeated eleven or thirteen times. An increase in the size of the octapeptide repeat leads to the production of an abnormally long version of PrP. It is unclear how the abnormal protein damages and ultimately destroys neurons, leading to the characteristic features of HDL 1.

### 2. Prion Disease

More than 30 mutations in the PRNP gene have been identified in people with familial forms of prion disease including, but not limited to, Creutzfeldt-Jakob disease (CJD), Gerstmann-Straussler-Scheinker syndrome (GSS), and/or fatal familial insomnia (FFI). The major features of these diseases include, but are not limited to, changes in memory, personality, and behavior; a decline in intellectual function (dementia); and abnormal movements, particularly difficulty with coordinating movements (ataxia). The signs and symptoms generally worsen over time, and can be fatal.

Some of the PRNP gene mutations that cause familial prion disease change single amino acids in PrP. Other mutations insert additional amino acids into the protein or result in an unusually short version of the protein. These changes alter the structure of PrP, leading to the production of an abnormally shaped protein, known as PrP^{Sc}, from one copy of the PRNP gene. In a process that is not fully understood, PrP^{Sc} can attach (bind) to PrP^{C} and promote its transformation into PrP^{Sc}. The abnormal protein builds up in the brain, forming clumps that damage or destroy neurons. The loss of these cells creates microscopic sponge-like holes (vacuoles) in the brain, which leads to the signs and symptoms of prion disease.

Several common variations (polymorphisms) in the PRNP gene have been identified that affect single amino acids in PrP. These polymorphisms do not cause prion disease, but they may affect a person's risk of developing these disorders. Studies have focused on the effects of a polymorphism at position 129 of PrP. At this position, people can have either the amino acid methionine (Met) or the amino acid valine (Val) (e.g., Met129Val or M129V). Because people inherit one copy of the PRNP gene from each parent, at position 129 an individual can receive methionine from both parents (Met/Met), valine from both parents (Val/Val), or methionine from one parent and valine from the other (Met/Val).

The Met129Val polymorphism appears to influence the risk of developing prion disease. Most affected individuals have the same amino acid at position 129 (Met/Met or Val/Val) instead of different amino acids (Met/Val). Having Met/Met at position 129 is also associated with an earlier age of onset and a more rapid worsening of the disease's signs and symptoms.

### 3. Wilson Disease

The PRNP Met129Val polymorphism has been reported to influence the onset of Wilson disease, an inherited disorder in which excessive amounts of copper accumulate in the body. While the primary cause of Wilson disease is an ATP7B gene mutation, symptoms of Wilson disease begin several years later in people who have Met/Met at position 129 in PrP as compared with those who have Met/Val or Val/Val. Other research findings indicate that this polymorphism may also affect the type of symptoms that develop in people with Wilson disease. Having Met/Met at position 129 appears to be associated with an increased occurrence of symptoms that affect the nervous system, particularly tremors.

### E. Iduronate 2-Sulfatase (IDS) Gene Diseases

The IDS gene encodes an enzyme called iduronate 2-sulfatase (I2S), which degrades large sugar molecules called glycosaminoglycans (GAGs). Specifically, I2S removes a sulfate from sulfated alpha-L-iduronic acid, which is present in GAGs such as heparan sulfate and dermatan sulfate. I2S is located in lysosomes, compartments within cells that digest and recycle different types of molecules.

### 1. Mucopolysaccharidosis Type II

More than 300 mutations in the IDS gene have been found to cause mucopolysaccharidosis type II (MPS II). Mutations that change one nucleotide are the most common. All presently known mutations that cause MPS II reduce or completely eliminate the function of I2S. It usually cannot be determined whether a certain mutation will cause severe or mild MPS II; however, mutations that result in the complete absence of I2S cause the more severe form of the disorder.

Lack of I2S enzyme activity leads to the accumulation of heparan sulfate and dermatan sulfate within cells, specifically inside the lysosomes. The buildup of these GAGs increases the size of the lysosomes, which is why many tissues and organs are enlarged in MPS II. It is believed that the accumulated GAGs may also interfere with the functions of other proteins inside the lysosomes and disrupt the movement of molecules inside the cell.

### F. Iduronidase, Alpha-L- (IDUA) Gene Diseases

The IDUA gene encodes an enzyme called alpha-L-iduronidase, which breaks down large sugar molecules called glycosaminoglycans (GAGs). Through a process called hydrolysis, alpha-L-iduronidase uses water molecules to break down unsulfated alpha-L-iduronic acid, which is present GAGs such as heparan sulfate and dermatan sulfate. Alpha-L-iduronidase is located in lysosomes, compartments within cells that digest and recycle different types of molecules.

### 1. Mucopolysaccharidosis Type I

More than 100 mutations in the IDUA gene have been found to cause mucopolysaccharidosis type I (MPS I). Mutations that change one DNA nucleotide are the most common. All presently known mutations that cause MPS I reduce or completely eliminate the function of alpha-L-iduronidase. It usually cannot be determined whether a certain mutation will cause severe or attenuated MPS I; however, people who do not produce any alpha-L-iduronidase have the severe form of this disorder.

The lack of alpha-L-iduronidase enzyme activity leads to the accumulation of heparan sulfate and dermatan sulfate within the lysosomes. The buildup of these GAGs increases the size of the lysosomes, which is why many tissues and organs are enlarged in MPS I. Researchers believe that the accumulated GAGs may also interfere with the functions of other proteins inside the lysosomes and disrupt the movement of molecules inside the cell.

### VII. cPE System Pharmaceutical Delivery Systems

The present invention contemplates several delivery systems for cPE systems that provide for roughly uniform distribution, have controllable rates of release. A variety of different media are described below that are useful in creating such delivery systems. It is not intended that any one medium or carrier is limiting to the present invention. Note that any medium or carrier may be combined with another medium or carrier.

Carriers or mediums contemplated by this invention comprise a material selected from the group comprising gelatin, collagen, cellulose esters, dextran sulfate, pentosan polysulfate, chitin, saccharides, albumin, fibrin sealants, synthetic polyvinyl pyrrolidone, polyethylene oxide, polypropylene oxide, block polymers of polyethylene oxide and polypropylene oxide, polyethylene glycol, acrylates, acrylamides, methacrylates including, but not limited to, 2-hydroxyethyl methacrylate, poly(ortho esters), cyanoacrylates, gelatin-resorcin-aldehyde type bioadhesives, polyacrylic acid and copolymers and block copolymers thereof.

One embodiment of the present invention contemplates an sPE delivery system further comprising a supplemental therapeutic agent

### A. Ribonucleoprotein (RNP) Nucleotransfection

In one embodiment, the present invention contemplates cPE system mRNA delivery. Although it is not necessary to understand the mechanism of an invention, it is believed that delivery of two smaller modular PE mRNAs (e.g., a Cas9/RT mRNA and a pegRNA or petRNA) would improve overall stability and large scale manufacturing efficiency as opposed to full length split PE fusion constructs that are approximately 6-7 kb length. Commercial translation of a full length split PE fusion construct is also problematic due to its small size. Consequently, RNP compositions comprising sPE RNA systems (e.g., nSpy Cas9 RNA+MCP-fused reverse transcriptase) provides both manufacturing and clinical advantages. In one embodiment, an RNP composition comprising sPE RNA systems are administered using ribonucleotransfection.

To efficiently transport CRISPR-Cas into target tissues/cells require overcoming several extra- and intra-cellular barriers, therefore largely limiting the applications of CRISPR-based therapeutics in vivo. Suggested delivery platforms include, but are not limited to, plasmids, RNAs and ribonucleoproteins (RNPs).

RNPs are composed of a large Cas protein and a short gRNA. gRNA can bind to DNA via Watson-Crick base pairing or the Cas protein can be conjugated to polypeptides, proteins, and PEI. These features can also be used for loading RNP. In addition, RNP can be loaded via electrostatic interactions with positively charged materials due to its negative net charge. These positively charged materials can be cationic lipids, PEI, polypeptides, and metal-organic frameworks (MOFs). Vesicles from cells can also be used to deliver RNP. It has been reported that PEI can coat a complex of Cas9 RNP and DNA nanoclews for enhanced endosomal escape. PEI-coated DNA nanoclews were shown to efficiently transfect a Cas9 RNP targeting EGFP into U2OS cells for EGFP knockout in vitro. Furthermore, the PEI-coated DNA nanoclews could also disrupt EGFP in U2OS.EGFP xenograft tumors in vivo after intratumoral injection. Recently, a nanocapsule was developed for Cas9 RNP delivery. Due to the heterogeneous surface charges of RNP, the RNP was first coated with both cationic and anionic monomers via electrostatic interactions. An imidazole-containing monomer (e.g., glutathione (GSH)-degradable crosslinker) and, PEG can be absorbed to the surface of the RNP via hydrogen bonding and van der Waals interactions. Then, GSH-cleavable nanocapsules were formed around the RNP via in situ free-radical polymerization. In addition, targeting ligands, for example CPPs, can be added into the nanocapsule by conjugation to PEG. It was demonstrated that the GSH cleavable nanocapsule could protect Cas9 RNP in the endosome after cellular uptake and could be quickly cleaved by GSH after escape into the cytoplasm for subsequent genome editing. After local injection of Cas9 RNP nanocapsules, robust gene editing was observed in retinal pigment epithelium (RPE) and muscle. Because the net charge of RNP is negative, cationic liposomes or LNPs can be directly used for RNP transfection. It was demonstrated that the Cas9 protein (+22 net charges) can be rendered highly anionic by fusion to a negatively charged GFP (−30 net charges) or complexation with a gRNA. Alternatively, the positively charged PEI has also been developed for RNP delivery. For example, Cas9 RNP was loaded onto GO-PEG-PEI via physisorption and n-stacking interactions. Xu et al., “Rational designs of in vivo CRISPR-Cas delivery systems” Adv Drug Deliv Rev (2021).

RNP delivery for genome editing in live cells may be performed with Lipofectamine® RNAiMAX lipid transfection reagent and elements of an sPE system. For example, pegRNAs/petRNAs are mixed with purified Cas9/RT proteins at an equimolar ratio in Opti-MEM™ to from an RNP complex (e.g, ˜10 min at room temperature). These RNPs can then be transfected into live cells using, for example, DMEM with 10% FBS. RNP nucleotransfection may be performed by electroporation using, for example, a Lonza 96-well Shuttle™ System (Lonza, Basel, Switzerland) optionally in the presence of Alt-R® Cas9 Electroporation Enhancer (Integrated DNA Technologies, Inc). Vakulskas et al., “A high-fidelity Cas9 mutant delivered as a ribonucleoprotein complex enables efficient gene editing in human haematopoietic stem and progenitor cells” Nat Med. 24(8): 1216-1224 (2018).

The prime editing efficiency of a number of genes was compared between PE3 and sPE in HEK293T cells using either conventional mRNA delivery or RNP-mediated nucleofection. For example, the genes included FANCF, VEGFA and HEK3.

### B. Microparticles

One embodiment of the present invention contemplates a medium comprising a microparticle. Preferably, microparticles comprise liposomes, nanoparticles, microspheres, nanospheres, microcapsules, and nanocapsules. Preferably, some microparticles contemplated by the present invention comprise poly(lactide-co-glycolide), aliphatic polyesters including, but not limited to, poly-glycolic acid and poly-lactic acid, hyaluronic acid, modified polysaccharides, chitosan, cellulose, dextran, polyurethanes, polyacrylic acids, pseudo-poly(amino acids), polyhydroxybutrate-related copolymers, polyanhydrides, polymethylmethacrylate, poly(ethylene oxide), lecithin and phospholipids.

### C. Liposomes

One embodiment of the present invention contemplates liposomes capable of attaching and releasing therapeutic agents described herein. Liposomes are microscopic spherical lipid bilayers surrounding an aqueous core that are made from amphiphilic molecules such as phospholipids. For example, a liposome may trap a therapeutic agent between the hydrophobic tails of the phospholipid micelle. Water soluble agents can be entrapped in the core and lipid-soluble agents can be dissolved in the shell-like bilayer. Liposomes have a special characteristic in that they enable water soluble and water insoluble chemicals to be used together in a medium without the use of surfactants or other emulsifiers. Liposomes can form spontaneously by forcefully mixing phospholipids in aqueous media. Water soluble compounds are dissolved in an aqueous solution capable of hydrating phospholipids. Upon formation of the liposomes, therefore, these compounds are trapped within the aqueous liposomal center. The liposome wall, being a phospholipid membrane, holds fat soluble materials such as oils. Liposomes provide controlled release of incorporated compounds. In addition, liposomes can be coated with water soluble polymers, such as polyethylene glycol to increase the pharmacokinetic half-life. One embodiment of the present invention contemplates an ultra high-shear technology to refine liposome production, resulting in stable, unilamellar (single layer) liposomes having specifically designed structural characteristics. These unique properties of liposomes, allow the simultaneous storage of normally immiscible compounds and the capability of their controlled release.

In some embodiments, the present invention contemplates cationic and anionic liposomes, as well as liposomes having neutral lipids. Preferably, cationic liposomes comprise negatively-charged materials by mixing the materials and fatty acid liposomal elements and allowing them to charge-associate. Clearly, the choice of a cationic or anionic liposome depends upon the desired pH of the final liposome mixture. Examples of cationic liposomes include lipofectin, lipofectamine, and lipofectace.

One embodiment of the present invention contemplates a medium comprising liposomes that provide controlled release of at least one therapeutic agent. Preferably, liposomes that are capable of controlled release: i) are biodegradable and non-toxic; ii) carry both water and oil soluble compounds; iii) solubilize recalcitrant compounds; iv) prevent compound oxidation; v) promote protein stabilization; vi) control hydration; vii) control compound release by variations in bilayer composition such as, but not limited to, fatty acid chain length, fatty acid lipid composition, relative amounts of saturated and unsaturated fatty acids, and physical configuration; viii) have solvent dependency; iv) have pH-dependency and v) have temperature dependency.

The compositions of liposomes are broadly categorized into two classifications. Conventional liposomes are generally mixtures of stabilized natural lecithin (PC) that may comprise synthetic identical-chain phospholipids that may or may not contain glycolipids. Special liposomes may comprise: i) bipolar fatty acids; ii) the ability to attach antibodies for tissue-targeted therapies; iii) coated with materials such as, but not limited to lipoprotein and carbohydrate; iv) multiple encapsulation and v) emulsion compatibility.

Liposomes may be easily made in the laboratory by methods such as, but not limited to, sonication and vibration. Alternatively, compound-delivery liposomes are commercially available. For example, Collaborative Laboratories, Inc. are known to manufacture custom designed liposomes for specific delivery requirements.

### D. Microspheres, Microparticles And Microcapsules

Microspheres and microcapsules are useful due to their ability to maintain a generally uniform distribution, provide stable controlled compound release and are economical to produce and dispense. Preferably, an associated delivery gel or the compound-impregnated gel is clear or, alternatively, said gel is colored for easy visualization by medical personnel.

Microspheres are obtainable commercially (Prolease®, Alkerme's: Cambridge, Mass.). For example, a freeze dried medium comprising at least one therapeutic agent is homogenized in a suitable solvent and sprayed to manufacture microspheres in the range of 20 to 90 μm. Techniques are then followed that maintain sustained release integrity during phases of purification, encapsulation and storage. Scott et al., Improving Protein Therapeutics With Sustained Release Formulations, Nature Biotechnology, Volume 16:153-157 (1998).

Modification of the microsphere composition by the use of biodegradable polymers can provide an ability to control the rate of therapeutic agent release. Miller et al., Degradation Rates of Oral Resorbable Implants {Polylactates and Polyglycolates: Rate Modification and Changes in PLA/PGA Copolymer Ratios, J. Biomed. Mater. Res., Vol. I1:711-719 (1977).

Alternatively, a sustained or controlled release microsphere preparation is prepared using an in-water drying method, where an organic solvent solution of a biodegradable polymer metal salt is first prepared. Subsequently, a dissolved or dispersed medium of a therapeutic agent is added to the biodegradable polymer metal salt solution. The weight ratio of a therapeutic agent to the biodegradable polymer metal salt may for example be about 1:100000 to about 1:1, preferably about 1:20000 to about 1:500 and more preferably about 1:10000 to about 1:500. Next, the organic solvent solution containing the biodegradable polymer metal salt and therapeutic agent is poured into an aqueous phase to prepare an oil/water emulsion. The solvent in the oil phase is then evaporated off to provide microspheres. Finally, these microspheres are then recovered, washed and lyophilized. Thereafter, the microspheres may be heated under reduced pressure to remove the residual water and organic solvent.

Other methods useful in producing microspheres that are compatible with a biodegradable polymer metal salt and therapeutic agent mixture are: i) phase separation during a gradual addition of a coacervating agent; ii) an in-water drying method or phase separation method, where an antiflocculant is added to prevent particle agglomeration and iii) by a spray-drying method.

In one embodiment, the present invention contemplates a medium comprising a microsphere or microcapsule capable of delivering a controlled release of a therapeutic agent for a duration of approximately between 1 day and 6 months. In one embodiment, the microsphere or microparticle may be colored to allow the medical practitioner the ability to see the medium clearly as it is dispensed. In another embodiment, the microsphere or microcapsule may be clear. In another embodiment, the microsphere or microparticle is impregnated with a radio-opaque fluoroscopic dye.

Controlled release microcapsules may be produced by using known encapsulation techniques such as centrifugal extrusion, pan coating and air suspension. Such microspheres and/or microcapsules can be engineered to achieve desired release rates. For example, Oliosphere® (Macromed) is a controlled release microsphere system. These particular microsphere's are available in uniform sizes ranging between 5-500 μm and composed of biocompatible and biodegradable polymers. Specific polymer compositions of a microsphere can control the therapeutic agent release rate such that custom-designed microspheres are possible, including effective management of the burst effect. ProMaxx® (Epic Therapeutics, Inc.) is a protein-matrix delivery system. The system is aqueous in nature and is adaptable to standard pharmaceutical delivery models. In particular, ProMaxx® are bioerodible protein microspheres that deliver both small and macromolecular drugs, and may be customized regarding both microsphere size and desired release characteristics.

In one embodiment, a microsphere or microparticle comprises a pH sensitive encapsulation material that is stable at a pH less than the pH of the internal mesentery. The typical range in the internal mesentery is pH 7.6 to pH 7.2. Consequently, the microcapsules should be maintained at a pH of less than 7. However, if pH variability is expected, the pH sensitive material can be selected based on the different pH criteria needed for the dissolution of the microcapsules. The encapsulated compound, therefore, will be selected for the pH environment in which dissolution is desired and stored in a pH preselected to maintain stability. Examples of pH sensitive material useful as encapsulants are Eudragit® L-100 or S-100 (Rohm GMBH), hydroxypropyl methylcellulose phthalate, hydroxypropyl methylcellulose acetate succinate, polyvinyl acetate phthalate, cellulose acetate phthalate, and cellulose acetate trimellitate. In one embodiment, lipids comprise the inner coating of the microcapsules. In these compositions, these lipids may be, but are not limited to, partial esters of fatty acids and hexitiol anhydrides, and edible fats such as triglycerides. Lew C. W., Controlled-Release pH Sensitive Capsule And Adhesive System And Method. U.S. Pat. No. 5,364,634 (herein incorporated by reference).

In one embodiment, the present invention contemplates a microparticle comprising a gelatin, or other polymeric cation having a similar charge density to gelatin (i.e., poly-L-lysine) and is used as a complex to form a primary microparticle. A primary microparticle is produced as a mixture of the following composition: i) Gelatin (60 bloom, type A from porcine skin), ii) chondroitin 4-sulfate (0.005%-0.1%), iii) glutaraldehyde (25%, grade 1), and iv) 1-ethyl-3-(3-dimethylaminopropyl)-carbodiimide hydrochloride (EDC hydrochloride), and ultra-pure sucrose (Sigma Chemical Co., St. Louis, Mo.). The source of gelatin is not thought to be critical; it can be from bovine, porcine, human, or other animal source. Typically, the polymeric cation is between 19,000-30,000 daltons. Chondroitin sulfate is then added to the complex with sodium sulfate, or ethanol as a coacervation agent.

Following the formation of a microparticle, a therapeutic agent is directly bound to the surface of the microparticle or is indirectly attached using a “bridge” or “spacer”. The amino groups of the gelatin lysine groups are easily derivatized to provide sites for direct coupling of a compound. Alternatively, spacers (i.e., linking molecules and derivatizing moieties on targeting ligands) such as avidin-biotin are also useful to indirectly couple targeting ligands to the microparticles. Stability of the microparticle is controlled by the amount of glutaraldehyde-spacer crosslinking induced by the EDC hydrochloride. A controlled release medium is also empirically determined by the final density of glutaraldehyde-spacer crosslinks.

In one embodiment, the present invention contemplates microparticles formed by spray-drying a composition comprising fibrinogen or thrombin with a therapeutic agent. Preferably, these microparticles are soluble and the selected protein (i.e., fibrinogen or thrombin) creates the walls of the microparticles. Consequently, the therapeutic agents are incorporated within, and between, the protein walls of the microparticle. Heath et al., Microparticles And Their Use In Wound Therapy. U.S. Pat. No. 6,113,948 (herein incorporated by reference). Following the application of the microparticles to living tissue, the subsequent reaction between the fibrinogen and thrombin creates a tissue sealant thereby releasing the incorporated compound into the immediate surrounding area.

One having skill in the art will understand that the shape of the microspheres need not be exactly spherical; only as very small particles capable of being sprayed or spread into or onto a surgical site (i.e., either open or closed). In one embodiment, microparticles are comprised of a biocompatible and/or biodegradable material selected from the group consisting of polylactide, polyglycolide and copolymers of lactide/glycolide (PLGA), hyaluronic acid, modified polysaccharides and any other well known material.

### VIII. cPE Administration Pharmaceutical Compositions

The present invention further provides pharmaceutical compositions comprising modular cPE systems as described herein. The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer, intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of elements that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the elements and the consistency of the final product.

Agents that enhance uptake of oligonucleotides at the cellular level may also be added to the pharmaceutical and other compositions of the present invention. For example, cationic lipids, such as lipofectin (U.S. Pat. No. 5,705,188), cationic glycerol derivatives, and polycationic molecules, such as polylysine (WO 97/30731), also enhance the cellular uptake of oligonucleotides.

The compositions of the present invention may additionally contain other adjunct elements conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the elements of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models or based on the examples described herein. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the compound is administered in maintenance doses, ranging from 0.01 μg to 100 g per kg of body weight, once or more daily, to once every 20 years.

### EXPERIMENTAL

### Example I

### Plasmids

PegRNA expression plasmids were constructed by using a custom vector (BfuAI- and EcoRI173 digested). Liu, P. et al., “Improved prime editors enable pathogenic allele correction and cancer modelling in adult mice” Nat Commun 12 (2021). The gBlock fragments with or without MS2 sequences were synthesized by Integrated DNA Technologies (IDT), followed by Gibson assembly using Gibson Assembly® Master Mix (New England Biolabs, NEB). Colonies were selected and confirmed by Sanger sequencing using a commercial human U6 primer. Nicking sgRNA plasmids were generated by annealing oligos and inserting them into the pMD217 vector digested by BfuAI.

The MS2 bacteriophage coat protein (MCP) and the Moloney Murine Leukemia Virus Reverse Transcriptase (M-MLV RT) has been described elsewhere. Konermann et al., “Genome-scale transcriptional activation by an engineered CRISPR-Cas9 complex” Nature 517:583-588 (2015); and Peabody, D. S., “The RNA binding site of bacteriophage MS2 coat protein” EMBO 324 J 12:595-600 (1993). Briefly, the sequence of M-MLV Reverse transcriptase (RT) was derived from a PE2 construct via PCR. The vector was prepared by excising nCas9 through dual digestion of PE2 plasmids with NotI and KpnI. Two gBlock gene fragments were synthesized for fusing MCP and M-MLV RT partially together with either a 32-amino-acid linker or an NLS linker, followed by Gibson assembly. The ligation mixtures were transformed into competent HB101 cells. The colonies were selected and confirmed by Sanger sequencing.

The scFv-RT plasmids were constructed by replacing nCas9 with the scFv fragment in PE2. The scFv sequence was derived from Addgene #60904 via PCR21, followed by Gibson assembly. The 3×Flag-RT plasmid was constructed by excising nCas9. The 3×Flag sequence was derived from Addgene #80456 via PCR. The DNA fragments were assembled by Gibson assembly.

Gene fragments of alternative RTs along with their bridging fragments were synthesized by Genewiz. Prime editors with alternative RTs were constructed by Gibson Assembly with PE2 digested by EcoRI and BsmI. Split alternative RT plasmids were modified from the 3×Flag-RT plasmid by digesting with NotI and EcoRI followed by Gibson Assembly with appropriate gene fragments.

The ribozyme-flanked RNA expression plasmids were constructed by replacing the pegRNA sequence from the U6-driven plasmid using Gibson Assembly with synthesized ribozyme fragments and a fragment containing the transcript to be circularized flanked by the hairpin sequences for circularization.

Plasmids were purified by Miniprep Kit (QIAGEN) or ZymoPURE™ II Plasmids Miniprep Kit for in vitro experiments.

### Example II

### Cell Culture Conditions

HEK293T, HeLa, A549, MDA-MB-231, U2OS, and IMR90 cells were acquired from ATCC and cultured in Dulbecco's modified Eagle medium (DMEM) supplemented with 10% (v/v) FBS (Gibco) and 1× penicillin-streptomycin (Corning). All cell lines were cultured and incubated at 37° C. and 5% CO2. All cell lines were authenticated by their suppliers and tested negative for Mycoplasma.

The mCherry reporter line and GFP reporter lines have been described elsewhere. Wang et al., “sgBE: a structure-guided design of sgRNA architecture specifies base editing window and enables simultaneous conversion of cytosine and adenosine” Genome Biol 21:222 (2020). Cells were cultured at 37° C. and 5% CO₂.

### Example III

mRNA Production

To construct the IVT templates, a CleanCap Reagent AG-compatible T7 promoter

(TAATACGACTCACTATAAG)

and a 5′ UTR were inserted at the 5′ of the Kozak sequence of the coding sequence in the mammalian expression vectors for nCas9, MMLV-RT and PE2. Additionally, a 3′ UTR, a 110-nt poly(A) tract and a restriction site (BsmBI) were inserted after the stop codon. Plasmids were completely linearized using BsmBI (New England Biolabs) before being used in the IVT, which was carried out at 37° C. using the HiScrib T7 High Yield RNA Synthesis Kit (New England Biolabs) with the addition of CleanCap Reagent AG (Trilink Biotechnologies) and with a 100% replacement of UTP by N1-Methylpseudo-UTP (Trilink Biotechnologies). The reaction was terminated after 4 h by a 15-min incubation with DNase I (New England Biolabs) added. The RNA was then purified using Monarch RNA Cleanup Kit (New England Biolabs).

### Example IV

### Reverse Transcriptase Production

For bacterial expression of MMLV-RT protein, the coding sequence was cloned into a pET28a vector and transformed into BL21 (DE3) Rosetta competent cells (Novagen) and selected on Luria broth (LB) agar plates containing kanamycin (Kan) and chloramphenicol (Cam). One liter of LB+Kan+Cam media was inoculated at 37° C. by 4 ml of overnight culture from a single colony. The culture was induced with 1 mM IPTG at OD₆₀₀ 0.8 for 3 h at 37° C. The pellet was washed with washed with 1×PBS and snap-frozen using liquid nitrogen.

For purification of MMLV-RT protein, the cell pellet was resuspended in Lysis Buffer (50 mM Tris-HCl, pH 8.0, 100 mM NaCl, 10 mM imidazole, 1 mM DTT and 0.1% Triton X-100) and incubated on ice for 30 min with lysozyme added to a final concentration of 1 mg/ml. The cells were lysed by using an EpiShea Probe Sonicator (Active Motif) and cleared by centrifuge at 20,000 g for 30 min at 4° C., before being loaded on a column with Ni-NTA resin pre-equilibrated with Wash Buffer I (50 mM Tris-HCl, pH 8.0, 500 mM NaCl, 10 mM imidazole, 1 mM DTT and 0.1% Triton X-100). The resin was washed by 10 column volume (CV) Wash Buffer I and 10 CV Wash Buffer II (50 mM Tris-HCl, pH 8.0, 100 mM NaCl, 10 mM imidazole and 1 mM DTT), before the protein was eluted using elution buffer (50 mM Tris-HCl, pH 8.0, 100 mM NaCl, 250 mM imidazole and 1 mM DTT). The eluate was dialyzed against Buffer A (50 mM Tris-HCl, pH 8.0, 100 mM NaCl, 0.1 mM EDTA and 5 mM DTT) overnight before being loaded onto a HiTrap SP HP cation exchange chromatography column (Cytiva). The column was washed with 10 CV Buffer A before the protein was eluted with a 0-100% gradient of Buffer B (50 mM Tris-HCl, pH 8.0, 1 M NaCl, 0.1 mM EDTA and 5 mM DTT). The peak fractions were pooled and buffer exchanged with Storage Buffer (50 mM Tris-HCl, pH 8.0, 100 mM NaCl, 0.1 mM EDTA and 5 mM DTT and 30% glycerol).

### Example V

RNP and mRNA Nucleofection

For nucleofections of mRNA and RNP, the Neon electroporation system was used. For mRNA nucleofection, 1 μg of each mRNA, 120 picomoles of pegRNA (Integrated DNA Technologies), 40 picomoles of nicking guide (Integrated DNA Technologies) and 50,000 HEK293T cells were mixed in buffer R and electroporated using 10 μL Neon tips, with electroporation parameters as follows: 1150 V, 20 ms, 2 pulses. After electroporation, cells were plated in pre-warmed 48-well plates with DMEM media containing 10% FBS and incubated for 72 h before analysis. For RNP nucleofection, same conditions were used except that the mRNAs were replaced by 61 picomoles of nCas9 protein (Alt-R S.p. Cas9 H840A Nickase V3, purchased from Integrated DNA Technologies) and 150 picomoles of NLS-containing MMLV-RT protein produced in-house.

The nucleofection protocol has been described elsewhere. 8. Briefly, all nucleofection was performed using the Neon electroporation system. For nucleofection, 1 μg of each mRNA, 100 pmol of pegRNA or LPET or DPET, 100 pmol of sgRNA, 100 pmol of nicking sgRNA, and 50,000 cells were mixed in Buffer R and electroporated with 10-μl Neon tips. The following electroporation parameters were used:

HEK293T, 1,150 V, 20 ms, two pulses.

A549, 1,200 V, 30 ms, two pulses.

HeLa, 1,005 V, 35 ms, two pulses.

MDA-MB-231, 1,400 V, 10 ms, four pulses.

U2OS, 1,400 V, 15 ms, four pulses.

IMR90, 1,600 V, 30 ms, 1 pulse.

After electroporation, cells were seeded in prewarmed 48-well plates with DMEM containing 10% FBS and incubated for 72 h before genomic DNA isolation. For genomic DNA isolation, cells were collected and lysed by 30 μl of Quick extraction buffer (Lucigen). Subsequently, the lysates were incubated in a thermocycler at 65° C. for 15 min, and 98° C. for 5 min.

### Example VI

### Plasmid Lipofection

Transfection of the plasmids in HEK293T cells were carried out according to the manufacturer's instructions for the Lipofectamine 3000 reagent (Invitrogen, #L3000015). Briefly, 1×10⁵ cells were seeded per well in a 12-well plate overnight. Cells were transfected using 3 μl Lipofectamine 3000 and P3000 (2 μl/μg DNA). For each well, 330 ng pegRNA, 110 ng nicking sgRNA, and 1 μg PE2 plasmids were used. The same amount of plasmids were used for modular sPE groups. After 72 hours post-transfection, cells were harvested and lysed using 100 μl Quick extraction buffer (Lucigene). Subsequently, the lysis was incubated on a PCR machine with 65° C. incubation for 15 min, 98° C. for 5 min.

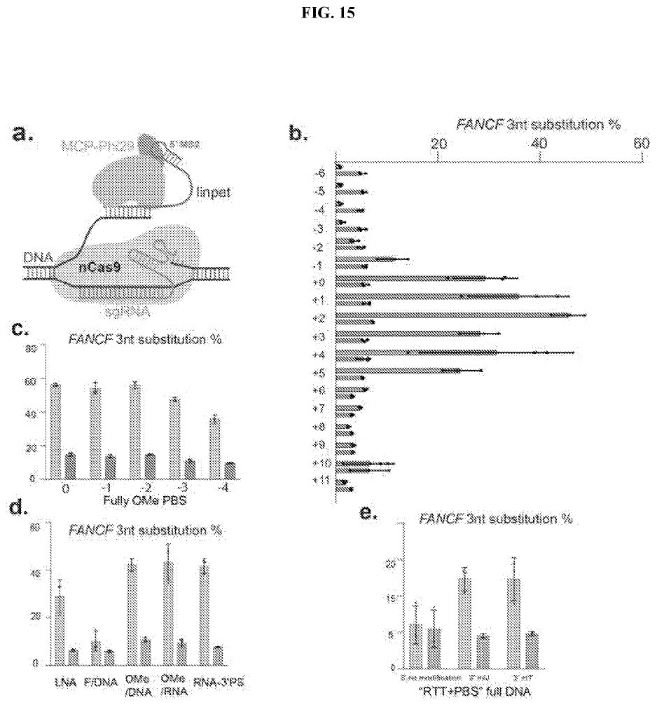

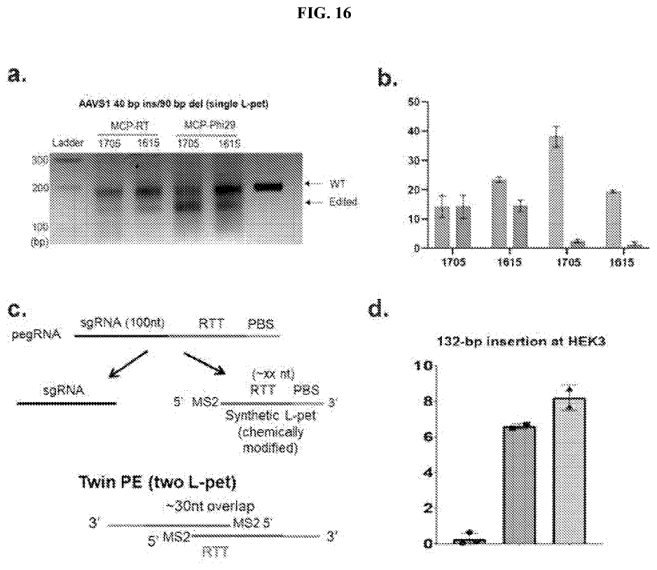

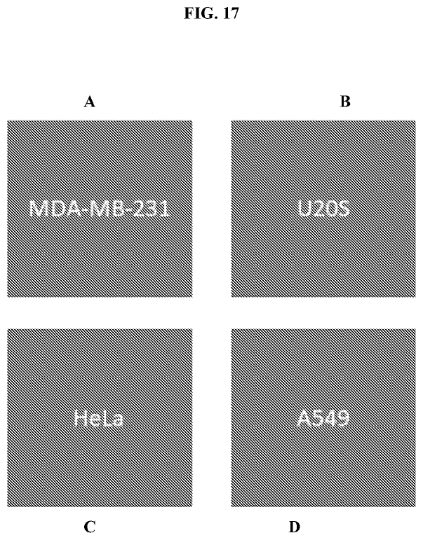

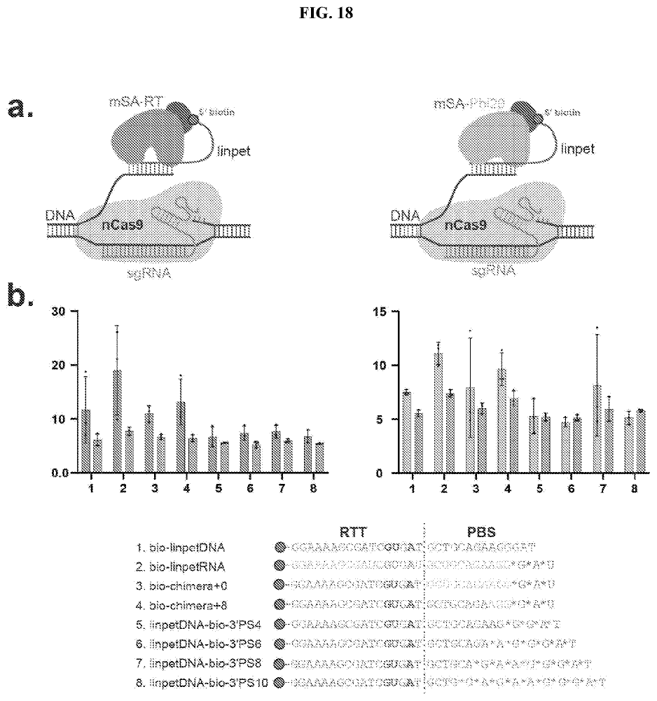

The integrity and abundance of the transiently expressed pegRNAs and petRNAs were assessed in Northern blots with multiple probes 72 h post-transfection in HEK293T cells. Probes were labeled using 32P-ATP and T4 PNK. Total RNAs containing the petRNA were loaded 10 times less than those without to allow better comparison between levels of pegRNA and petRNA when using the RTT-PBS probe. See, FIG. 15. Putative cleavage products are labeled by asterisks. For the last sample, RNase H and RNase R were used to demonstrate a circular nature of a petRNA.

### Example VII

### Mouse DNA Isolation Technique

Mouse genomic DNA was isolated using PureLink Genomic DNA Mini Kit (Thermo Fisher) according to the manufacturer's protocol.

### Example VIII

### Sanger Sequencing and Analysis

PCR amplification was performed around the target locus using Phusion Flash PCR Master Mix (Thermo Fisher) and specific primers. Sanger sequencing was performed by GENEWIZ (South Plainfield, NJ). The results were quantified using EditR22.

### Example XI

### Deep Sequencing and Data Analysis

Sequencing library preparation was done as described previously. Anzalone et al., “Search-and-replace genome editing without double-strand breaks or donor DNA”. Nature 576:149-157 (2019). Briefly, for the first round of PCR, specific primers carrying Illumina forward and reverse adapters were used for amplifying the genomic sites of interest with Phusion Hot Start II PCR Master Mix.

For the second-round PCR, primers containing unique Illumina barcodes were used. PCR reactions were performed as follows: 98° C. for 10 s, then (98° C. for 1 s, 55° C. for 5 s, and 72° C. for 6 s) for 20 cycles, followed by 72° C. extensions for 2 min as a final extension. The DNA products of second-round PCR were collected and purified by gel purification using the QIAquick Gel Extraction Kit (Qiagen). DNA concentration was determined by Qubit dsDNA HS Assay. Subsequently, the library was sequenced on an Illumina MiniSeq following the manufacturer's protocols.

MiniSeq sequencing reads were demultiplexed with bcl2fastq (Illumina). Editing efficiency was determined via aligning amplicon reads to a reference sequence through CRISPResso2 using HDR mode with the parameters ‘default_min_aln_score’=90, ‘min_average_read_quality’=30 The quantification window is ±20 bp around the sgRNA/pegRNA cut site8. The background substitution frequencies were ignored as putative sequencing errors or subtracted from the triplicate negative control group (when available)20. The percentage of ‘HDR’ in the CRISPResso report represented the percentage of the desired edits. The percentage of indels was determined as the sum of the percentages of those ‘Imperfect HDR’, ‘NHEJ’ and ‘Ambiguous’, groups according to the report.

### Example X

### Flow Cytometry Analysis

Flow cytometry analysis was performed on day 3 after transfection. mCherry or GFP reporter lines were harvested after PBS washing and 0.25% trypsin digestion, followed by re-centrifuging at 300×g for 5 min followed by resuspension in PBS with 2% FBS. The proportions of GFP and/or mCherry-positive cells were quantified using flow cytometry (MACSQuant VYB). Data were analysed by FlowJo v10 software.

### Example XI

### Animals

All mice studies were approved by the Institutional Animal Care and Use Committee (IACUC) at UMass medical school. All plasmids were prepared using an Endo-Free-Maxi kit (Qiagen) and delivered by hydrodynamic tail-vein injection. For cancer model generation, eight-week-old 246 FVB/NJ mice (Strain #001800) were injected with 30 μg PE2 or split Cas9 nickase and RT, 15 μg pegRNA, 15 μg sgRNA nicking, 5 μg pT3 EF1a-MYC (Addgene #92046) and 1 μg CMV SB10 (Addgene #24551) via the tail vein.

### Example XII

### Histology and Immunohistochemistry

The procedure of IHC staining has been described²⁴. Briefly, livers were fixed with 4% formalin overnight, embedded with paraffin, and sectioned at 5 μM, followed by hematoxylin and eosin (H&E) staining for pathology. Liver sections were de-waxed, rehydrated, and stained according to previous immunohistochemistry protocols²⁵. The following antibody was used: β-CATENIN (BD, 610154, 1:200). The images were captured by Leica DMi8 microscopy.

### Example XIII

### Gene Editing Protocol of Disease Modifying Loci

Cell transfection is according to the manufacturer's instructions for the Lipofectamine 3000 reagent (Invitrogen, #L3000015). Briefly, 1×105 cells were seeded per well in a 12-well plate overnight. Cells were transfected using 3 μl Lipofectamine 3000 and P3000 (2 μl/μg DNA). For each well, 330 ng pegRNA, 110 ng nicking sgRNA, and 1 μg PE2 plasmids were used. The same amount of plasmids were used for sPE groups. After 72 hours post-transfection, cells were harvested and lysed using 100 μl Quick extraction buffer (Lucigene). Subsequently, the lysis was incubated on a PCR machine with 65° C. incubation for 15 min, 98° C. for 5 min.

### Example IVX

In Vivo Gene Editing with Modular sPE

AAV vectors (AAV8 capsids) were packaged and produced at the Viral Vector Core of the Horae Gene Therapy Center, University of Massachusetts Medical School. Virus titers were measured by gel electrophoresis followed by silver staining and ddPCR.

For AAV injection, 1×10¹² GC AAV-Cas9^{H840A} and 1×10¹² GC AAV-U6-pegRNA-U6-sgRNA-M-MLV were resuspended in 200 μL 0.9% sodium chloride and administered via tail vein injection

### Example XV

### Oligonucleotide Synthesis

Synthetic pegRNAs, sgRNAs, nicking gRNAs, and all-RNA LPETs were obtained from Integrated DNA Technologies (Supplementary Table 1 and Supplementary Table 2) as Alt-R RNAs, consistent with previous studies8.

LPET and DPET (Supplementary Table 1 and Supplementary Table 2) oligonucleotides were synthesized at 1 μmole scale on a Biolytic Dr. Oligo 48 Medium Throughput Oligo Synthesizer. Similar methods have been described elsewhere 30. BMT (0.25 M in acetonitrile, TEDIA) was used as activator, and 0.05 M iodine in pyridine:water (9:1) (TEDIA) was used as oxidizer. DTTT (0.1 M, ChemGenes) was used as sulfurizing agent. A total of 3% TCA in DCM (TEDIA) was used as deblock solution. Oligonucleotides were synthesized on 1000 Å CPG functionalized with the first nucleobase amidite in the sequence (˜42 μmol/g). RNA, DNA, and 2′-OMe phosphoramidites (ChemGenes) were dissolved in acetonitrile to 0.15 M; the coupling time was 10 min for each base. The nucleobase protecting groups were removed with a 1:1 NH4OH: 40% aqueous methylamine solution for 15 min at 65° C. Deprotection of the TBDMS group was achieved with DMSO:NEt3·3HF (4:1) solution (500 μL) at 65° C. for 2 h. Oligonucleotides were then recovered by precipitation in cold isopropanol. The pellet was then washed with 1M NaOAc in 70% EtOH and resuspended in 1 mL RNase-free water.

Purification of oligonucleotides was carried out by high performance liquid chromatography using a 1260 infinity system with an Agilent PL-SAX 1000 Å column (150×7.5 mm, 8 μm). Buffer A: 30% acetonitrile in water; Buffer B: 30% acetonitrile in 1 M NaClO4 (aq). Excess salt was removed with a Sephadex Nap-10 column.

Oligonucleotides were analyzed on an Agilent 6530 Q-TOF LC/MS system with electrospray ionization and time-of-flight ion separation in negative ionization mode. Liquid chromatography was performed using a 2.1×50-mm AdvanceBio oligonucleotide column (Agilent Technologies, Santa Clara, CA). The data were analyzed using Agilent Mass Hunter software. Buffer A: 100 mM hexafluoroisopropanol with 9 mM triethylamine in water; Buffer B: 100 mM hexafluoroisopropanol with 9 mM trimethylamine in methanol.

### Example XVI

### Cell Viability

The CellTiter-Glo luminescent cell viability kit (Promega Corporation, Madison, WI, USA) was used to examine cell viability and cytotoxicity. After electroporation with indicated mRNAs following the nucleofection protocol, HEK293T cells were seeded in prewarmed 48-well plates with DMEM containing 10% FBS and incubated with indicated time. Afterwards, the cells were trypsinized, transferred to a 96-well plate, and incubated following the CellTiter-Glo luminescent cell viability protocol. The intensity of luminescence was detected by plate reader.

### Example XVII

### Sanger Sequencing

PCR amplification was performed with specific primers around the target locus using Phusion Flash PCR Master Mix (Thermo Fisher). Subsequently, DNA products were purified by QIAquick PCR Purification Kit or QIAquick Gel Extraction Kit. Sanger sequencing was performed by Genewiz.

### Example XVIII

Droplet Digital Polymerase Chain Reaction (ddPCR)

A single-amplicon, dual-probe Taqman ddPCR approach was used to quantitate the efficiency of precision editing outcomes. Briefly, a locus-specific probe (HEX) and an editing-specific probe (FAM) were designed within the same amplicon encompassing the genomic site of interest (Supplemental Table 4). gDNA was mixed with the ddPCR Supermix for Probes (no dUTP) (Bio-Rad), along with primers (900 nM) and probes (250 nM), in a final volume of 20 μL. Droplet generation was done by using a QX200 Manual Droplet Generator (Bio-Rad). The PCR program used is as follows: 95° C. for 10 min, 35 cycles of 94° C. for 30 s and 58° C. for 1 min, 98° C. for 10 min and 4° C. hold. Data acquisition was performed on a QX200 Droplet Reader (Bio-Rad). Data analysis was done using QuantaSoft (Bio-Rad). The editing efficiency was calculated as follows: editing %=100%*FAM+/HEX+.

TABLE II
Synthetic sgRNAs, nicking sgRNAs, and pegRNAs.

| Syntenic | |
|---|---|
| sgRNA | |
| and | |
| pegRNA | sequence (5′-3′) |
| FANCF | mG*mG*mA*rArUrCrCrCrUrUrCrUrGrCrArGrCrArCrCrGrUrUrUrUrArGrArGrCrUrArGrArArArUrArG |
| sgRNA | rCrArArGrUrUrArArArArUrArArGrGrCrUrArGrUrCrCrGrUrUrArUrCrArArCrUrUrGrArArArArArGr |
| | UrGrGrGrArCrCrGrArGrUrCrGrGrUrCrCmU*mU*mU*rU |
| FANCF | mG*mG*mG*rGrUrCrCrCrArGrGrUrGrCrUrGrArCrGrUrGrUrUrUrUrArGrArGrCrUrArGrArArArUrArG |
| nicking | rCrArArGrUrUrArArArArUrArArGrGrCrUrArGrUrCrCrGrUrUrArUrCrArArCrUrUrGrArArArArArGr |
| sgRNA | UrGrGrCrArCrCrGrArGrUrCrGrGrUrGrCmU*mU*mU*rU |
| IDS | mG*mC*mA*rUrUrUrUrCrGrArUrUrCrCrGrUrGrArCrUrGrUrUrUrUrArGrArGrCrUrArGrArArArUrArG |
| sgRNA | rCrArArGrUrUrArArArArUrArArGrGrCrUrArGrUrCrCrGrUrUrArUrCrArArCrUrUrGrArArArArArGr |
| | UrGrGrGrArCrCrGrArGrUrCrGrGrUrCrCmU*mU*mU*rU |
| IDS | mA*mC*mU*rGrArGrGrGrArUrGrUrCrUrGrArArGrGrCrGrUrUrUrUrArGrArGrCrUrArGrArArArUrArG |
| nicking | rCrArArGrUrUrArArArArUrArArGrGrCrUrArGrUrCrCrGrUrUrArUrCrArArCrUrUrGrArArArArArGr |
| sgRNA | UrGrGrGrArCrCrGrArGrUrCrGrGrUrCrOmU*mU*mU*rU |
| PRNP | mG*mC*mA*rGrUrGrGrUrGrGrGrGrGrGrCrCrUrUrGrGrGrUrUrUrUrArGrArGrCrUrArGrArArArUrArG |
| sgRNA | rCrArArGrUrUrArArArArUrArArGrGrCrUrArGrUrCrCrGrUrUrArUrCrArArCrUrUrGrArArArArArGr |
| | UrGrGrCrArCrCrGrArGrUrCrGrGrUrGrCmU*mU*mU*rU |
| PRNP | mG*mC*mA*rUrGrUrUrUrUrCrArCrGrArUrArGrUrArArGrUrUrUrUrArGrArGrCrUrArGrArArArUrArG |
| nicking | rCrArArGrUrUrArArArArUrArArGrGrCrUrArGrUrCrCrGrUrUrArUrCrArArCrUrUrGrArArArArArGr |
| sgRNA | UrGrGrCrArCrCrGrArGrUrCrGrGrUrGrCmU*mU*mU*rU |
| HEK3 | mG*mG*mC*rCrCrArGrArCrUrGrArGrCrArCrGrUrGrArGrUrUrUrUrArGrArGrCrUrArGrArArArUrArG |
| sgRNA | rCrArArGrUrUrArArArArUrArArGrGrCrUrArGrUrCrCrGrUrUrArUrCrArArCrUrUrGrArArArArArGr |
| | UrGrGrCrArCrCrGrArGrUrCrGrGrUrGrCmU*mU*mU*rU |
| HEK3 | mG*mU*mC*rArArCrCrArGrUrArUrCrCrCrGrGrUrGrCrGrUrUrUrUrArGrArGrCrUrArGrArArArUrArG |
| nicking | rCrArArGrUrUrArArArArUrArArGrGrCrUrArGrUrCrCrGrUrUrArUrCrArArCrUrUrGrArArArArArGr |
| sgRNA | UrGrGrCrArCrCrGrArGrUrCrGrGrUrGrCmU*mU*mU*rU |
| AAVS1 | mG*mC*mA*rGrCrUrCrArGrGrUrUrCrUrGrGrGrArGrArGrUrUrUrUrArGrArGrCrUrArGrArArArUrArG |
| sgRNA | rCrArArGrUrUrArArArArUrArArGrGrCrUrArGrUrCrCrGrUrUrArUrCrArArCrUrUrGrArArArArArGr |
| | UrGrGrGrArCrCrGrArGrUrCrGrGrUrCrC*mU*mU*mUrU |
| AAVS1 | mG*mA*mU*rGrGrArGrCrCrArGrArGrArGrGrArUrCrCrGrUrUrUrUrArGrArGrCrUrArGrArArArUrArG |
| nicking | rCrArArGrUrUrArArArArUrArArGrGrCrUrArGrUrCrCrGrUrUrArUrCrArArCrUrUrGrArArArArArGr |
| sgRNA | UrGrGrGrArCrCrGrArGrUrCrGrGrUrCrC*mU*mU*mUrU |
| HBB | mC*mA*mU*rGrGrUrGrCrArCrCrUrGrArCrUrCrCrUrGrGrUrUrUrUrArGrArGrCrUrArGrArArArUrArG |
| sgRNA | rCrArArGrUrUrArArArArUrArArGrGrCrUrArGrUrCrCrGrUrUrArUrCrArArCrUrUrGrArArArArArGr |
| | UrGrGrCrArCrCrGrArGrUrCrGrG*mU*mG*mC |
| HBB | mC*mc*mU*rUrGrArUrArCrCrArArCrCrUrGrCrCrCrArGrUrUrUrUrArGrArGrCrUrArGrArArArUrArG |
| nicking | rCrArArGrUrUrArArArArUrArArGrGrCrUrArGrUrCrCrGrUrUrArUrCrArArCrUrUrGrArArArArArGr |
| sgRNA | UrGrGrCrArCrCrGrArGrUrCrGrG*mU*mG*mC |
| RUNX1 | mG*mC*mA*rUrUrUrUrCrArGrGrArGrGrArArGrCrGrArGrUrUrUrUrArGrArGrCrUrArGrArArArUrArG |
| sgRNA | rCrArArGrUrUrArArArArUrArArGrGrCrUrArGrUrCrCrGrUrUrArUrCrArArCrUrUrGrArArArArArGr |
| | UrGrGrCrArCrCrGrArGrUrCrGrG*mU*mG*mC |
| RUNX1 | mA*mU*mG*rArArGrCrArCrUrGrUrGrGrGrUrArCrGrArGrUrUrUrUrArGrArGrCrUrArGrArArArUrArG |
| nicking | rCrArArGrUrUrArArArArUrArArGrGrCrUrArGrUrCrCrGrUrUrArUrCrArArCrUrUrGrArArArArArGr |
| sgRNA | UrGrGrCrArCrCrGrArGrUrCrGrGrUrGrCmU*mU*mU*rU |
| FANCF | mG*mG*mA*rArUrCrCrCrUrUrCrUrGrCrArGrCrArCrCrGrUrUrUrUrArGrArGrCrUrArGrArArArUrArG |
| pegRNA | rCrArArGrUrUrArArArArUrArArGrGrCrUrArGrUrCrCrGrUrUrArUrCrArArCrUrUrGrArArArArArGr |
| | UrGrGrGrArCrCrGrArGrUrCrGrGrUrCrCrGrGrArArArArGrCrGrArUrCrGrUrGrArUrGrCrUrGrCrArG |
| | rArArGrG*mG*mA*mU |
| PRNP | mG*mC*mA*rGrUrGrGrUrGrGrGrGrGrGrCrCrUrUrGrGrGrUrUrUrUrArGrArGrCrUrArGrArArArUrArG |
| pegRNA | rCrArArGrUrUrArArArArUrArArGrGrCrUrArGrUrCrCrGrUrUrArUrCrArArCrUrUrGrArArArArArGr |
| | UrGrGrGrArCrCrGrArGrUrCrGrGrUrCrCrArUrGrUrArGrArCrGrCrCrArArGrGrCrCrCrCrCrCrArCrC |
| | mU*mU*mU*rU |
| RUNX1 | mG*mC*mA*rUrUrUrUrCrArGrGrArGrGrArArGrCrGrArGrUrUrUrUrArGrArGrCrUrArGrArArArUrArG |
| pegRNA | rCrArArGrUrUrArArArArUrArArGrGrCrUrArGrUrCrCrGrUrUrArUrCrArArCrUrUrGrArArArArArGr |
| | UrGrGrGrArCrCrGrArGrUrCrGrGrUrCrCrUrGrUrCrUrGrArArGrCrCrArUrCrCrArUrGrCrUrUrCrCrU |
| | rCrCrUrGrArArArArUmU*mU*mU*rU |
| mCherry | mA*mA*mG*rUrUrCrArGrCrGrUrGrUrCrCrGrGrCrUrUrGrUrUrUrUrArGrArGrCrUrArGrArArArUrArG |
| sgRNA | rCrArArGrUrUrArArArArUrArArGrGrCrUrArGrUrCrCrGrUrUrArUrCrArArCrUrUrGrArArArArArGr |
| | UrGrGrGrArCrCrGrArGrUrCrGrG*mU*mC*mC |
| mCherry | mG*mU*mA*rGrGrUrCrArGrGrGrUrGrGrUrCrArCrGrArGrUrUrUrUrArGrArGrCrUrArGrArArArUrArG |
| nicking | rCrArArGrUrUrArArArArUrArArGrGrCrUrArGrUrCrCrGrUrUrArUrCrArArCrUrUrGrArArArArArGr |
| sgRNA | UrGrGrGrArCrCrGrArGrUrCrGrG*mU*mC*mc |

TABLE III
Synthetic LPETs and DPETs.

| | sequence (5′-3′) |
|---|---|
| L-pet | (DNA/RNA) |
| FANCF L-petRNA | [symbol] |
| | [symbol] |
| FANCF chimera −6 | [symbol] |
| | [symbol] |
| FANCF chimera −5 | [symbol] |
| | [symbol] |
| FANCF chimera −4 | [symbol] |
| | [symbol] |
| FANCF chimera −3 | [symbol] |
| | [symbol] |
| FANCF chimera −2 | [symbol] |
| FANCF chimera −1 | [symbol] |
| FANCF chimera +0 | [symbol] |
| FANCF chimera +1 | [symbol] |
| FANCF chimera +2 | [symbol] |
| FANCF chimera +3 | [symbol] |
| FANCF chimera +4 | [symbol] |
| FANCF chimera +5 | [symbol] |
| FANCF chimera +6 | [symbol] |
| FANCF chimera +7 | [symbol] |
| FANCF chimera +8 | [symbol] |
| FANCF chimera +9 | [symbol] |
| FANCF MS2-less | [symbol] |
| LPET(+2) | |
| FANCF MS2-less | [symbol] |
| ssDNA | |
| FANCF unmod | [symbol] |
| petRNA | [symbol] |
| FANCF chimera +10 | [symbol] |
| FANCF chimera +11 | [symbol] |
| FANCF fully OMe | [symbol] |
| PBS + 0 | |
| FANCF fully OMe | [symbol] |
| PBS +1 | |
| FANCF fully OMe | [symbol] |
| PBS +2 | |
| FANCF fully OMe | [symbol] |
| PBS +3 | |
| FANCF fully OMe | [symbol] |
| PBS +4 | |
| FANCF OMe/DNA alt | [symbol] |
| 1 | [symbol] |
| FANCF F/DNA alt 1 | [symbol] |
| | [symbol] |
| FANCF chimera +3 | [symbol] |
| OMe/RNA alt 1 | |
| FANCF chimera +3 | [symbol] |
| RNA 3′PS alt 1 | |
| FANCF chimera with | [symbol] |
| LNA | |
| FANCF 3′ no | [symbol] |
| modification | |
| FANCF 3′ mU | [symbol] |
| FANCF 3′ *T | [symbol] |
| FANCF bio-L- | [symbol] |
| petDNA | |
| FANCF bio-L- | [symbol] |
| petDNA | |
| +0 | |
| FANOF bic-L- | [symbol] |
| petDNA | |
| +8 | |
| FANCF L-petDNA- | [symbol] |
| bio-3′PS4 | |
| FANCF L-petDNA- | [symbol] |
| bio-3′PS6 | |
| FANOF L-petDNA- | [symbol] |
| bio-3′PS8 | |
| FANCF L-petDNA- | [symbol] |
| bio-3′PS10 | |
| PRNP L-petRNA | [symbol] |
| | C |
| PRNP L-pet-chimera | [symbol] |
| −6 | |
| PRNP L-pet-chimera | [symbol] |
| −3 | |
| PRNP L-pet- | [symbol] |
| chimera +0 | |
| PRNP L-pet-chimera | [symbol] |
| +2 | |
| PRNP L-pet-chimera | [symbol] |
| +4 | |
| PRNP L-pet-chimera | [symbol] |
| +6 | |
| PRNP L-pet-chimera | [symbol] |
| +8 | |
| IDS L-petRNA | [symbol] |
| | [symbol] |
| IDS L-pet-Chimera | [symbol] |
| +0 | |
| HBB L-pet +2 | [symbol] |
| RUNX1 L-pet +2 | [symbol] |
| PRNP L-pet | [symbol] |
| HBB bio-L-pet | [symbol] |
| AAVS1-loxP-1615 | [symbol] |
| | [symbol] |
| AAVS1-loxP-1705 | [symbol] |
| | [symbol] |
| HEK3-twin-LPET-1 | [symbol] |
| | [symbol] |
| HEK3-twin-LPET-2 | [symbol] |
| | [symbol] |
| mCherry L-pet | [symbol] |
| mCherry bio-L-pet | /5BiosG/ [symbol] |

[symbol]

indicates data missing or illegible when filed

TABLE 4
Primers used for high throughput sequencing.

| Gene | | |
|---|---|---|
| locus | F (5′-3′) | R (5′-3′) |
| FANCF- | CTACACGACGCTCTTCCGATCTGATGGATGTGGCGCAGG | AGACGTGTGCTCTTCCGATCTAGGCGTATCATT |
| 1 | TAG | TCGCGGAT |
| FANCF- | CTACACGACGCTCTTCCGATCTGACCAAAGCGCCGATGG | AGACGTGTGCTCTTCCGATCTGGTGAAAGCGG |
| 2 | AT | AAGTAGGGC |
| DS | CTACACGACGCTCTTCCGATCTGTGCGTATGGAATAGCCC | AGACGTGTGCTCTTCCGATCTACGTTGAGCTGT |
| | ATGATC | GCAGAGAAG |
| PRNP | CTACACGACGCTCTTCCGATCTAGTAAGCCAAAAACCAAC | AGACGTGTGCTCTTCCGATCTCTGTACTCATCC |
| | ATGAAGC | ATGGGCCTG |
| RUNX1 | ACATGAAGCCTACACGACGCTCTTCCGATCTTCGCTCCGA | AGACGTGTGCTCTTCCGATCTCAGGCAAAGCT |
| | AGGTAAAAGAAATC | GAGCAAAAGTAG |
| HBB | CTACACGACGCTCTTCCGATCTTTGCTTACATTTGCTTCTG | AGACGTGTGCTCTTCCGATCTTCTGTCTCCACA |
| | ACACAAC | TGCCCAG |
| AAVS1 | CTACACGACGCTCTTCCGATCTCCAGGATCAGTGAAACGC | AGACGTGTGCTCTTCCGATCTCTTGCCAGAACC |
| | AC | TCTAAGGT |

TABLE 5
Primers used for droplet digital PCR (ddPCR).

| Gene | |
|---|---|
| locus | F (5′-3′) |
| HEK3 | Wt probe cctggcctgggtcaatccttgg |
| HEK3 | ins probe cggcgacgtaaacggccac |
| HEK3 | caccaccccggtgaacagctc |
| HEK3 | f gcatgcatttgtaggcttgatg |
| HEK3 | r cagccaaacttgtcaaccag |

1. A high fidelity chimeric prime editing system, comprising:
i) a Cas9 nickase;
ii) a high fidelity Phi29 nucleotide polymerase protein (HFPhi29NTPol);
iii) a single guide RNA, and
iv) a chimeric prime editor template oligonucleotide comprising a ribonucleic acid region that at least partially encodes a primer binding site (PBS) and a deoxyribonucleic acid region that at least partially encodes a nucleotide polymerase template (NPT).

2. The system of claim 1, wherein said deoxyribonucleic acid region further at least partially encodes said PBS.

3. The system of claim 1, wherein said ribonucleic acid region further at least partially encodes said NPT.

4. The system of claim 1, wherein said ribonucleic acid region comprises a 3′-terminus with at least one chemical modification.

5. The system of claim 1, wherein said deoxyribonucleic acid region comprises a 5′-terminus appended to a ribonucleic acid stem loop.

6. The system of claim 5, wherein said ribonucleic acid stem loop is an MS2 stem loop.

7-31. (canceled)

32. A composition, comprising:
a) a ribonucleic acid (RNA) delivery system; and
b) a high fidelity chimeric prime editing system comprising:
i) a first ribonucleic acid (RNA) encoding a Cas9 nickase protein and a high fidelity Phi29 nucleotide polymerase protein (HFPhi29NTPol); and
ii) a second RNA encoding a single guide RNA and a chimeric prime editor template oligonucleotide comprising a ribonucleic acid region that at least partially encodes a primer binding site (PBS) and a deoxyribonucleic acid region that at least partially encodes a nucleotide polymerase template (NPT).

33. The composition of claim 32, wherein said deoxyribonucleic acid region further at least partially encodes said PBS.

34. The composition of claim 32, wherein said ribonucleic acid region further at least partially encodes said NPT.

35. The composition of claim 32, wherein said deoxyribonucleic acid region comprises a 5′-terminus appended to a ribonucleic acid stem loop.

36. The composition of claim 35, wherein said ribonucleic acid stem loop is an MS2 stem loop.

37. The composition of claim 32, wherein said ribonucleic acid region comprises a 3′-terminus with at least one chemical modification.

38. The composition of claim 32, wherein said HFPhi29NTPol is a high fidelity deoxyribonucleotide polymerase.

39-51. (canceled)

52. A method, comprising;
a) providing;
i) a patient expressing at least one symptom of a genetic disease or disorder; and
ii) a pharmaceutically acceptable composition comprising;
A) a ribonucleic acid (RNA) delivery system; and
B) a high fidelity chimeric prime editing system comprising a first ribonucleic acid (RNA) encoding a Cas9 nickase protein and a high fidelity Phi29 nucleotide polymerase protein (HFPhi29NTPol), and a second RNA encoding a single guide RNA and a chimeric prime editor template oligonucleotide comprising a ribonucleic acid region that at least partially encodes a primer binding site (PBS) and a deoxyribonucleic acid region that at least partially encodes a nucleotide polymerase template (NPT); and
b) administering said pharmaceutically acceptable composition to said patient, wherein said at least one symptom of said genetic disease or disorder is reduced.

53. The method of claim 52, wherein said deoxyribonucleic acid region further at least partially encodes said PBS.

54. The method of claim 52, wherein said ribonucleic acid region further at least partially encodes said NPT.

55. The method of claim 52, wherein said deoxyribonucleic acid region comprises a 5′-terminus appended to a ribonucleic acid stem loop.

56. The method of claim 55, wherein said ribonucleic acid stem loop is an MS2 stem loop.

57. The method of claim 52, wherein said ribonucleic acid region comprises a 3′-terminus with at least one chemical modification.

58. The method of claim 52, wherein said HFPhi29NTPol is a high fidelity deoxyribonucleotide polymerase.

59-162. (canceled)


SEQUENCE LISTING (219 sequences)

SEQ ID NO: 1 - DNA, 30 bp, synthetic construct
  source 1..30: mol_type = other DNA
gcagtggtggggggccttggcggctacatg

SEQ ID NO: 2 - DNA, 30 bp, synthetic construct
  source 1..30: mol_type = other DNA
gcagtggtggggggccttggcgtctacatg

SEQ ID NO: 3 - DNA, 30 bp, synthetic construct
  source 1..30: mol_type = other DNA
actgagggatgtctgaaggccggggatact

SEQ ID NO: 4 - DNA, 30 bp, synthetic construct
  source 1..30: mol_type = other DNA
actgagggatgtctgaaggccagggatact

SEQ ID NO: 5 - DNA, 19 bp, synthetic construct
  source 1..19: mol_type = other DNA
taatacgactcactataag

SEQ ID NO: 6 - RNA, 100 nt, synthetic construct
  source 1..100: mol_type = other RNA
  modified_base 1..3: mod_base = OTHER; note = The residue in this position is a 2-O-methylated ribonucleotide.
  modified_base 97..99: mod_base = OTHER; note = The residue in this position is a 2-O-methylated ribonucleotide.
  modified_base 1..3: mod_base = OTHER; note = The residue in this position has a 3-phosphorothioate.
  modified_base 97..99: mod_base = OTHER; note = The residue in this position has a 3-phosphorothioate.
ggaatcccttctgcagcaccgttttagagctagaaatagcaagttaaaataaggctagtccgttatcaacttgaaaaagtgggaccgagtcggtcctttt

SEQ ID NO: 7 - RNA, 100 nt, synthetic construct
  source 1..100: mol_type = other RNA
  modified_base 1..3: mod_base = OTHER; note = The residue in this position is a 2-O-methylated ribonucleotide
  modified_base 97..99: mod_base = OTHER; note = The residue in this position is a 2-O-methylated ribonucleotide
  modified_base 1..3: mod_base = OTHER; note = The residue in this position has a 3-phosphorothioate.
  modified_base 97..99: mod_base = OTHER; note = The residue in this position has a 3-phosphorothioate.
ggggtcccaggtgctgacgtgttttagagctagaaatagcaagttaaaataaggctagtccgttatcaacttgaaaaagtggcaccgagtcggtgctttt

SEQ ID NO: 8 - RNA, 100 nt, synthetic construct
  source 1..100: mol_type = other RNA
  modified_base 1..3: mod_base = OTHER; note = The residue in this position is a 2-O-methylated ribonucleotide.
  modified_base 97..99: mod_base = OTHER; note = The residue in this position is a 2-O-methylated ribonucleotide.
  modified_base 1..3: mod_base = OTHER; note = The residue in this position has a 3-phosphorothioate.
  modified_base 97..99: mod_base = OTHER; note = The residue in this position has a 3-phosphorothioate.
gcattttcgattccgtgactgttttagagctagaaatagcaagttaaaataaggctagtccgttatcaacttgaaaaagtgggaccgagtcggtcctttt

SEQ ID NO: 9 - RNA, 100 nt, synthetic construct
  source 1..100: mol_type = other RNA
  modified_base 1..3: mod_base = OTHER; note = The residue in this position is a 2-O-methylated ribonucleotide.
  modified_base 97..99: mod_base = OTHER; note = The residue in this position is a 2-O-methylated ribonucleotide.
  modified_base 1..3: mod_base = OTHER; note = The residue in this position has a 3-phosphorothioate.
  modified_base 97..99: mod_base = OTHER; note = The residue in this position has a 3-phosphorothioate.
actgagggatgtctgaaggcgttttagagctagaaatagcaagttaaaataaggctagtccgttatcaacttgaaaaagtgggaccgagtcggtcctttt

SEQ ID NO: 10 - RNA, 100 nt, synthetic construct
  source 1..100: mol_type = other RNA
  modified_base 1..3: mod_base = OTHER; note = The residue in this position is a 2-O-methylated ribonucleotide.
  modified_base 97..99: mod_base = OTHER; note = The residue in this position is a 2-O-methylated ribonucleotide.
  modified_base 1..3: mod_base = OTHER; note = The residue in this position has a 3-phosphorothioate.
  modified_base 97..99: mod_base = OTHER; note = The residue in this position has a 3-phosphorothioate.
gcagtggtggggggccttgggttttagagctagaaatagcaagttaaaataaggctagtccgttatcaacttgaaaaagtggcaccgagtcggtgctttt

SEQ ID NO: 11 - RNA, 100 nt, synthetic construct
  source 1..100: mol_type = other RNA
  modified_base 1..3: mod_base = OTHER; note = The residue in this position is a 2-O-methylated ribonucleotide.
  modified_base 97..99: mod_base = OTHER; note = The residue in this position is a 2-O-methylated ribonucleotide.
  modified_base 1..3: mod_base = OTHER; note = The residue in this position has a 3-phosphorothioate.
  modified_base 97..99: mod_base = OTHER; note = The residue in this position has a 3-phosphorothioate.
gcatgttttcacgatagtaagttttagagctagaaatagcaagttaaaataaggctagtccgttatcaacttgaaaaagtggcaccgagtcggtgctttt

SEQ ID NO: 12 - RNA, 100 nt, synthetic construct
  source 1..100: mol_type = other RNA
  modified_base 1..3: mod_base = OTHER; note = The residue in this position is a 2-O-methylated ribonucleotide.
  modified_base 97..99: mod_base = OTHER; note = The residue in this position is a 2-O-methylated ribonucleotide.
  modified_base 1..3: mod_base = OTHER; note = The residue in this position has a 3-phosphorothioate.
  modified_base 97..99: mod_base = OTHER; note = The residue in this position has a 3-phosphorothioate.
ggcccagactgagcacgtgagttttagagctagaaatagcaagttaaaataaggctagtccgttatcaacttgaaaaagtggcaccgagtcggtgctttt

SEQ ID NO: 13 - RNA, 100 nt, synthetic construct
  source 1..100: mol_type = other RNA
  modified_base 1..3: mod_base = OTHER; note = The residue in this position is a 2-O-methylated ribonucleotide.
  modified_base 97..99: mod_base = OTHER; note = The residue in this position is a 2-O-methylated ribonucleotide.
  modified_base 1..3: mod_base = OTHER; note = The residue in this position has a 3-phosphorothioate.
  modified_base 97..99: mod_base = OTHER; note = The residue in this position has a 3-phosphorothioate.
gtcaaccagtatcccggtgcgttttagagctagaaatagcaagttaaaataaggctagtccgttatcaacttgaaaaagtggcaccgagtcggtgctttt

SEQ ID NO: 14 - RNA, 100 nt, synthetic construct
  source 1..100: mol_type = other RNA
  modified_base 1..3: mod_base = OTHER; note = The residue in this position is a 2-O-methylated ribonucleotide.
  modified_base 97..99: mod_base = OTHER; note = The residue in this position is a 2-O-methylated ribonucleotide.
  modified_base 1..3: mod_base = OTHER; note = The residue in this position has a 3-phosphorothioate.
  modified_base 97..99: mod_base = OTHER; note = The residue in this position has a 3-phosphorothioate.
gcagctcaggttctgggagagttttagagctagaaatagcaagttaaaataaggctagtccgttatcaacttgaaaaagtgggaccgagtcggtcctttt

SEQ ID NO: 15 - RNA, 100 nt, synthetic construct
  source 1..100: mol_type = other RNA
  modified_base 1..3: mod_base = OTHER; note = The residue in this position is a 2-O-methylated ribonucleotide.
  modified_base 97..99: mod_base = OTHER; note = The residue in this position is a 2-O-methylated ribonucleotide.
  modified_base 1..3: mod_base = OTHER; note = The residue in this position has a 3-phosphorothioate.
  modified_base 97..99: mod_base = OTHER; note = The residue in this position has a 3-phosphorothioate.
gatggagccagagaggatccgttttagagctagaaatagcaagttaaaataaggctagtccgttatcaacttgaaaaagtgggaccgagtcggtcctttt

SEQ ID NO: 16 - RNA, 96 nt, synthetic construct
  source 1..96: mol_type = other RNA
  modified_base 1..3: mod_base = OTHER; note = The residue in this position is a 2-O-methylated ribonucleotide.
  modified_base 94..96: mod_base = OTHER; note = The residue in this position is a 2-O-methylated ribonucleotide.
  modified_base 1..3: mod_base = OTHER; note = The residue in this position has a 3-phosphorothioate.
  modified_base 94..96: mod_base = OTHER; note = The residue in this position has a 3-phosphorothioate.
catggtgcacctgactcctggttttagagctagaaatagcaagttaaaataaggctagtccgttatcaacttgaaaaagtggcaccgagtcggtgc

SEQ ID NO: 17 - RNA, 96 nt, synthetic construct
  source 1..96: mol_type = other RNA
  modified_base 1..3: mod_base = OTHER; note = The residue in this position is a 2-O-methylated ribonucleotide.
  modified_base 94..96: mod_base = OTHER; note = The residue in this position is a 2-O-methylated ribonucleotide.
  modified_base 1..3: mod_base = OTHER; note = The residue in this position has a 3-phosphorothioate.
  modified_base 94..96: mod_base = OTHER; note = The residue in this position has a 3-phosphorothioate.
ccttgataccaacctgcccagttttagagctagaaatagcaagttaaaataaggctagtccgttatcaacttgaaaaagtggcaccgagtcggtgc

SEQ ID NO: 18 - RNA, 96 nt, synthetic construct
  source 1..96: mol_type = other RNA
  modified_base 1..3: mod_base = OTHER; note = The residue in this position is a 2-O-methylated ribonucleotide.
  modified_base 94..96: mod_base = OTHER; note = The residue in this position is a 2-O-methylated ribonucleotide.
  modified_base 1..3: mod_base = OTHER; note = The residue in this position has a 3-phosphorothioate.
  modified_base 94..96: mod_base = OTHER; note = The residue in this position has a 3-phosphorothioate.
gcattttcaggaggaagcgagttttagagctagaaatagcaagttaaaataaggctagtccgttatcaacttgaaaaagtggcaccgagtcggtgc

SEQ ID NO: 19 - RNA, 100 nt, synthetic construct
  source 1..100: mol_type = other RNA
  modified_base 1..3: mod_base = OTHER; note = The residue in this position is a 2-O-methylated ribonucleotide.
  modified_base 97..99: mod_base = OTHER; note = The residue in this position is a 2-O-methylated ribonucleotide.
  modified_base 1..3: mod_base = OTHER; note = The residue in this position has a 3-phosphorothioate.
  modified_base 97..99: mod_base = OTHER; note = The residue in this position has a 3-phosphorothioate.
atgaagcactgtgggtacgagttttagagctagaaatagcaagttaaaataaggctagtccgttatcaacttgaaaaagtggcaccgagtcggtgctttt

SEQ ID NO: 20 - RNA, 127 nt, synthetic construct
  source 1..127: mol_type = other RNA
  modified_base 1..3: mod_base = OTHER; note = The residue in this position is a 2-O-methylated ribonucleotide.
  modified_base 125..127: mod_base = OTHER; note = The residue in this position is a 2-O-methylated ribonucleotide.
  modified_base 1..3: mod_base = OTHER; note = The residue in this position has a 3-phosphorothioate.
  modified_base 124..126: mod_base = OTHER; note = The residue in this position has a 3-phosphorothioate.
ggaatcccttctgcagcaccgttttagagctagaaatagcaagttaaaataaggctagtccgttatcaacttgaaaaagtgggaccgagtcggtccggaaaagcgatcgtgatgctgcagaagggat

SEQ ID NO: 21 - RNA, 124 nt, synthetic construct
  source 1..124: mol_type = other RNA
  modified_base 1..3: mod_base = OTHER; note = The residue in this position is a 2-O-methylated ribonucleotide.
  modified_base 121..123: mod_base = OTHER; note = The residue in this position is a 2-O-methylated ribonucleotide.
  modified_base 1..3: mod_base = OTHER; note = The residue in this position has a 3-phosphorothioate.
  modified_base 121..123: mod_base = OTHER; note = The residue in this position has a 3-phosphorothioate.
gcagtggtggggggccttgggttttagagctagaaatagcaagttaaaataaggctagtccgttatcaacttgaaaaagtgggaccgagtcggtccatgtagacgccaaggccccccacctttt

SEQ ID NO: 22 - RNA, 133 nt, synthetic construct
  source 1..133: mol_type = other RNA
  modified_base 1..3: mod_base = OTHER; note = The residue in this position is a 2-O-methylated ribonucleotide.
  modified_base 130..132: mod_base = OTHER; note = The residue in this position is a 2-O-methylated ribonucleotide.
  modified_base 1..3: mod_base = OTHER; note = The residue in this position has a 3-phosphorothioate.
  modified_base 130..132: mod_base = OTHER; note = The residue in this position has a 3-phosphorothioate.
gcattttcaggaggaagcgagttttagagctagaaatagcaagttaaaataaggctagtccgttatcaacttgaaaaagtgggaccgagtcggtcctgtctgaagccatccatgcttcctcctgaaaattttt

SEQ ID NO: 23 - RNA, 96 nt, synthetic construct
  source 1..96: mol_type = other RNA
  modified_base 1..3: mod_base = OTHER; note = The residue in this position is a 2-O-methylated ribonucleotide.
  modified_base 94..96: mod_base = OTHER; note = The residue in this position is a 2-O-methylated ribonucleotide.
  modified_base 1..3: mod_base = OTHER; note = The residue in this position has a 3-phosphorothioate.
  modified_base 93..95: mod_base = OTHER; note = The residue in this position has a 3-phosphorothioate.
aagttcagcgtgtccggcttgttttagagctagaaatagcaagttaaaataaggctagtccgttatcaacttgaaaaagtgggaccgagtcggtcc

SEQ ID NO: 24 - RNA, 96 nt, synthetic construct
  source 1..96: mol_type = other RNA
  modified_base 1..3: mod_base = OTHER; note = The residue in this position is a 2-O-methylated ribonucleotide.
  modified_base 94..96: mod_base = OTHER; note = The residue in this position is a 2-O-methylated ribonucleotide.
  modified_base 1..3: mod_base = OTHER; note = The residue in this position has a 3-phosphorothioate.
  modified_base 93..95: mod_base = OTHER; note = The residue in this position has a 3-phosphorothioate.
gtaggtcagggtggtcacgagttttagagctagaaatagcaagttaaaataaggctagtccgttatcaacttgaaaaagtgggaccgagtcggtcc

SEQ ID NO: 25 - RNA, 54 nt, synthetic construct
  source 1..54: mol_type = other RNA
  modified_base 1..3: mod_base = OTHER; note = The residue in this position is a 2-O-methylated ribonucleotide.
  modified_base 52..54: mod_base = OTHER; note = The residue in this position is a 2-O-methylated ribonucleotide.
  modified_base 1..3: mod_base = OTHER; note = The residue in this position has a 3-phosphorothioate.
  modified_base 51..53: mod_base = OTHER; note = The residue in this position has a 3-phosphorothioate.
gcacatgaggatcacccatgtgcggaaaagcgatcgtgatgctgcagaagggat

SEQ ID NO: 26 - RNA, 54 nt, synthetic construct; identical to SEQ ID NO: 25
  source 1..54: mol_type = other RNA
  modified_base 1..3: mod_base = OTHER; note = The residue in this position is a 2-O-methylated ribonucleotide.
  modified_base 24..34: mod_base = OTHER; note = The residues in these positions are DNA
  modified_base 52..54: mod_base = OTHER; note = The residue in this position is a 2-O-methylated ribonucleotide.
  modified_base 1..3: mod_base = OTHER; note = The residue in this position has a 3-phosphorothioate.
  modified_base 51..53: mod_base = OTHER; note = The residue in this position has a 3-phosphorothioate.

SEQ ID NO: 27 - RNA, 54 nt, synthetic construct; identical to SEQ ID NO: 25
  source 1..54: mol_type = other RNA
  modified_base 1..3: mod_base = OTHER; note = The residue in this position is a 2-O-methylated ribonucleotide.
  modified_base 52..54: mod_base = OTHER; note = The residue in this position is a 2-O-methylated ribonucleotide.
  modified_base 24..34: mod_base = OTHER; note = The residues in these positions are DNA.
  modified_base 1..3: mod_base = OTHER; note = The residue in this position has a 3-phosphorothioate.
  modified_base 51..53: mod_base = OTHER; note = The residue in this position has a 3-phosphorothioate.

SEQ ID NO: 28 - RNA, 54 nt, synthetic construct; identical to SEQ ID NO: 25
  source 1..54: mol_type = other RNA
  modified_base 1..3: mod_base = OTHER; note = The residue in this position is a 2-O-methylated ribonucleotide.
  modified_base 24..36: mod_base = OTHER; note = The residues in these positions are DNA
  modified_base 52..54: mod_base = OTHER; note = The residue in this position is a 2-O-methylated ribonucleotide.
  modified_base 1..3: mod_base = OTHER; note = The residue in this position has a 3-phosphorothioate.
  modified_base 51..53: mod_base = OTHER; note = The residue in this position has a 3-phosphorothioate.

SEQ ID NO: 29 - RNA, 54 nt, synthetic construct; identical to SEQ ID NO: 25
  source 1..54: mol_type = other RNA
  modified_base 1..3: mod_base = OTHER; note = The residue in this position is a 2-O-methylated ribonucleotide
  modified_base 24..36: mod_base = OTHER; note = The residue in this position is DNA
  modified_base 52..54: mod_base = OTHER; note = The residue in this position is a 2-O-methylated ribonucleotide
  modified_base 1..3: mod_base = OTHER; note = The residue in this position has a 3-phosphorothioate.
  modified_base 51..53: mod_base = OTHER; note = The residue in this position has a 3-phosphorothioate.

SEQ ID NO: 30 - RNA, 54 nt, synthetic construct; identical to SEQ ID NO: 25
  source 1..54: mol_type = other RNA
  modified_base 24..38: mod_base = OTHER; note = The residue in this position is DNA
  modified_base 1..3: mod_base = OTHER; note = The residue in this position is a 2-O-methylated ribonucleotide.
  modified_base 52..54: mod_base = OTHER; note = The residue in this position is a 2-O-methylated ribonucleotide.
  modified_base 1..3: mod_base = OTHER; note = The residue in this position has a 3-phosphorothioate.
  modified_base 51..53: mod_base = OTHER; note = The residue in this position has a 3-phosphorothioate.

SEQ ID NO: 31 - RNA, 54 nt, synthetic construct; identical to SEQ ID NO: 25
  source 1..54: mol_type = other RNA
  modified_base 1..3: mod_base = OTHER; note = The residue in this position is a 2-O-methylated ribonucleotide.
  modified_base 52..54: mod_base = OTHER; note = The residue in this position is a 2-O-methylated ribonucleotide.
  modified_base 24..39: mod_base = OTHER; note = The residue in this position is DNA
  modified_base 1..3: mod_base = OTHER; note = The residue in this position has a 3-phosphorothioate.
  modified_base 51..53: mod_base = OTHER; note = The residue in this position has a 3-phosphorothioate.

SEQ ID NO: 32 - RNA, 54 nt, synthetic construct; identical to SEQ ID NO: 25
  source 1..54: mol_type = other RNA
  modified_base 1..3: mod_base = OTHER; note = The residue in this position is a 2-O-methylated ribonucleotide.
  modified_base 52..54: mod_base = OTHER; note = The residue in this position is a 2-O-methylated ribonucleotide.
  modified_base 24..39: mod_base = OTHER; note = The residue in this position is DNA
  modified_base 1..3: mod_base = OTHER; note = The residue in this position has a 3-phosphorothioate.
  modified_base 51..53: mod_base = OTHER; note = The residue in this position has a 3-phosphorothioate.

SEQ ID NO: 33 - RNA, 54 nt, synthetic construct; identical to SEQ ID NO: 25
  source 1..54: mol_type = other RNA
  modified_base 1..3: mod_base = OTHER; note = The residue in this position is a 2-O-methylated ribonucleotide.
  modified_base 52..54: mod_base = OTHER; note = The residue in this position is a 2-O-methylated ribonucleotide.
  modified_base 24..41: mod_base = OTHER; note = The residue at this position is DNA
  modified_base 1..3: mod_base = OTHER; note = The residue in this position has a 3-phosphorothioate.
  modified_base 51..53: mod_base = OTHER; note = The residue in this position has a 3-phosphorothioate.

SEQ ID NO: 34 - RNA, 54 nt, synthetic construct; identical to SEQ ID NO: 25
  source 1..54: mol_type = other RNA
  modified_base 1..3: mod_base = OTHER; note = The residue in this position is a 2-O-methylated ribonucleotide.
  modified_base 52..54: mod_base = OTHER; note = The residue in this position is a 2-O-methylated ribonucleotide.
  modified_base 24..42: mod_base = OTHER; note = The residue in this position is DNA
  modified_base 1..3: mod_base = OTHER; note = The residue in this position has a 3-phosphorothioate.
  modified_base 51..53: mod_base = OTHER; note = The residue in this position has a 3-phosphorothioate.

SEQ ID NO: 35 - RNA, 54 nt, synthetic construct; identical to SEQ ID NO: 25
  source 1..54: mol_type = other RNA
  modified_base 1..3: mod_base = OTHER; note = The residue in this position is a 2-O-methylated ribonucleotide.
  modified_base 52..54: mod_base = OTHER; note = The residue in this position is a 2-O-methylated ribonucleotide.
  modified_base 24..43: mod_base = OTHER; note = The residue in this position is DNA
  modified_base 1..3: mod_base = OTHER; note = The residue in this position has a 3-phosphorothioate.
  modified_base 51..53: mod_base = OTHER; note = The residue in this position has a 3-phosphorothioate.

SEQ ID NO: 36 - RNA, 54 nt, synthetic construct; identical to SEQ ID NO: 25
  source 1..54: mol_type = other RNA
  modified_base 1..3: mod_base = OTHER; note = The residue in this position is a 2-O-methylated ribonucleotide.
  modified_base 52..54: mod_base = OTHER; note = The residue in this position is a 2-O-methylated ribonucleotide.
  modified_base 24..44: mod_base = OTHER; note = The residue in this position is DNA
  modified_base 1..3: mod_base = OTHER; note = The residue in this position has a 3-phosphorothioate.
  modified_base 51..53: mod_base = OTHER; note = The residue in this position has a 3-phosphorothioate.

SEQ ID NO: 37 - RNA, 54 nt, synthetic construct; identical to SEQ ID NO: 25
  source 1..54: mol_type = other RNA
  modified_base 1..3: mod_base = OTHER; note = The residue in this position is a 2-O-methylated ribonucleotide.
  modified_base 52..54: mod_base = OTHER; note = The residue in this position is a 2-O-methylated ribonucleotide.
  modified_base 24..45: mod_base = OTHER; note = The residue in this position is DNA
  modified_base 1..3: mod_base = OTHER; note = The residue in this position has a 3-phosphorothioate.
  modified_base 51..53: mod_base = OTHER; note = The residue in this position has a 3-phosphorothioate.

SEQ ID NO: 38 - RNA, 54 nt, synthetic construct; identical to SEQ ID NO: 25
  source 1..54: mol_type = other RNA
  modified_base 1..3: mod_base = OTHER; note = The residue in this position is a 2-O-methylated ribonucleotide.
  modified_base 52..54: mod_base = OTHER; note = The residue in this position is a 2-O-methylated ribonucleotide.
  modified_base 24..46: mod_base = OTHER; note = The residue in this position is DNA
  modified_base 1..3: mod_base = OTHER; note = The residue in this position has a 3-phosphorothioate.
  modified_base 51..53: mod_base = OTHER; note = The residue in this position has a 3-phosphorothioate.

SEQ ID NO: 39 - RNA, 54 nt, synthetic construct; identical to SEQ ID NO: 25
  source 1..54: mol_type = other RNA
  modified_base 1..3: mod_base = OTHER; note = The residue in this position is a 2-O-methylated ribonucleotide.
  modified_base 52..54: mod_base = OTHER; note = The residue in this position is a 2-O-methylated ribonucleotide.
  modified_base 24..47: mod_base = OTHER; note = The residue in this position is DNA
  modified_base 1..3: mod_base = OTHER; note = The residue in this position has a 3-phosphorothioate.
  modified_base 51..53: mod_base = OTHER; note = The residue in this position has a 3-phosphorothioate.

SEQ ID NO: 40 - RNA, 54 nt, synthetic construct; identical to SEQ ID NO: 25
  source 1..54: mol_type = other RNA
  modified_base 1..3: mod_base = OTHER; note = The residue in this position is a 2-O-methylated ribonucleotide.
  modified_base 52..54: mod_base = OTHER; note = The residue in this position is a 2-O-methylated ribonucleotide.
  modified_base 24..48: mod_base = OTHER; note = The residue at this position is DNA
  modified_base 1..3: mod_base = OTHER; note = The residue in this position has a 3-phosphorothioate.
  modified_base 51..53: mod_base = OTHER; note = The residue in this position has a 3-phosphorothioate.

SEQ ID NO: 41 - RNA, 54 nt, synthetic construct; identical to SEQ ID NO: 25
  source 1..54: mol_type = other RNA
  modified_base 1..3: mod_base = OTHER; note = The residue in this position is a 2-O-methylated ribonucleotide.
  modified_base 52..54: mod_base = OTHER; note = The residue in this position is a 2-O-methylated ribonucleotide.
  modified_base 24..49: mod_base = OTHER; note = The residue in this position is DNA
  modified_base 1..3: mod_base = OTHER; note = The residue in this position has a 3-phosphorothioate.
  modified_base 51..53: mod_base = OTHER; note = The residue in this position has a 3-phosphorothioate.

SEQ ID NO: 42 - RNA, 31 nt, synthetic construct
  source 1..31: mol_type = other RNA
  modified_base 1..19: mod_base = OTHER; note = The residue in this position is DNA
  modified_base 29..31: mod_base = OTHER; note = The residue in this position is a 2-O-methylated ribonucleotide.
  modified_base 28..30: mod_base = OTHER; note = The residue in this position has a 3-phosphorothioate.
ggaaaagcgatcgtgatgctgcagaagggat

SEQ ID NO: 43 - DNA, 31 bp, synthetic construct
  source 1..31: mol_type = other DNA
ggaaaagcgatcgtgatgctgcagaagggat

SEQ ID NO: 44 - RNA, 54 nt, synthetic construct; identical to SEQ ID NO: 25
  source 1..54: mol_type = other RNA

SEQ ID NO: 45 - RNA, 54 nt, synthetic construct; identical to SEQ ID NO: 25
  source 1..54: mol_type = other RNA
  modified_base 1..3: mod_base = OTHER; note = The residue in this position is a 2-O-methylated ribonucleotide.
  modified_base 52..54: mod_base = OTHER; note = The residue in this position is a 2-O-methylated ribonucleotide.
  modified_base 24..50: mod_base = OTHER; note = The residue in this position is DNA
  modified_base 1..3: mod_base = OTHER; note = The residue in this position has a 3-phosphorothioate.
  modified_base 51..53: mod_base = OTHER; note = The residue in this position has a 3-phosphorothioate.

SEQ ID NO: 46 - RNA, 54 nt, synthetic construct; identical to SEQ ID NO: 25
  source 1..54: mol_type = other RNA
  modified_base 1..3: mod_base = OTHER; note = The residue in this position is a 2-O-methylated ribonucleotide.
  modified_base 52..54: mod_base = OTHER; note = The residue in this position is a 2-O-methylated ribonucleotide.
  modified_base 24..51: mod_base = OTHER; note = The residue in this position is DNA
  modified_base 1..3: mod_base = OTHER; note = The residue in this position has a 3-phosphorothioate.
  modified_base 51..53: mod_base = OTHER; note = The residue in this position has a 3-phosphorothioate.

SEQ ID NO: 47 - RNA, 54 nt, synthetic construct; identical to SEQ ID NO: 25
  source 1..54: mol_type = other RNA
  modified_base 1..3: mod_base = OTHER; note = The residue in this position is a 2-O-methylated ribonucleotide.
  modified_base 41..54: mod_base = OTHER; note = The residue in this position is a 2-O-methylated ribonucleotide.
  modified_base 24..40: mod_base = OTHER; note = The residue in this position is DNA
  modified_base 1..3: mod_base = OTHER; note = The residue in this position has a 3-phosphorothioate.
  modified_base 51..53: mod_base = OTHER; note = The residue in this position has a 3-phosphorothioate.

SEQ ID NO: 48 - RNA, 54 nt, synthetic construct; identical to SEQ ID NO: 25
  source 1..54: mol_type = other RNA
  modified_base 1..3: mod_base = OTHER; note = The residue in this position is a 2-O-methylated ribonucleotide.
  modified_base 41..54: mod_base = OTHER; note = The residue in this position is a 2-O-methylated ribonucleotide.
  modified_base 24..41: mod_base = OTHER; note = The residue in this position is DNA
  modified_base 1..3: mod_base = OTHER; note = The residue in this position has a 3-phosphorothioate.
  modified_base 51..53: mod_base = OTHER; note = The residue in this position has a 3-phosphorothioate.

SEQ ID NO: 49 - RNA, 54 nt, synthetic construct; identical to SEQ ID NO: 25
  source 1..54: mol_type = other RNA
  modified_base 1..3: mod_base = OTHER; note = The residue in this position is a 2-O-methylated ribonucleotide.
  modified_base 42..54: mod_base = OTHER; note = The residue in this position is a 2-O-methylated ribonucleotide.
  modified_base 24..41: mod_base = OTHER; note = The residue in this position is DNA
  modified_base 1..3: mod_base = OTHER; note = The residue in this position has a 3-phosphorothioate.
  modified_base 51..53: mod_base = OTHER; note = The residue in this position has a 3-phosphorothioate.

SEQ ID NO: 50 - RNA, 54 nt, synthetic construct; identical to SEQ ID NO: 25
  source 1..54: mol_type = other RNA
  modified_base 1..3: mod_base = OTHER; note = The residue in this position is a 2-O-methylated ribonucleotide.
  modified_base 44..54: mod_base = OTHER; note = The residue in this position is a 2-O-methylated ribonucleotide.
  modified_base 24..43: mod_base = OTHER; note = The residue in this position is DNA
  modified_base 1..3: mod_base = OTHER; note = The residue in this position has a 3-phosphorothioate.
  modified_base 51..53: mod_base = OTHER; note = The residue in this position has a 3-phosphorothioate.

SEQ ID NO: 51 - RNA, 54 nt, synthetic construct; identical to SEQ ID NO: 25
  source 1..54: mol_type = other RNA
  modified_base 1..3: mod_base = OTHER; note = The residue in this position is a 2-O-methylated ribonucleotide.
  modified_base 45..54: mod_base = OTHER; note = The residue in this position is a 2-O-methylated ribonucleotide.
  modified_base 24..44: mod_base = OTHER; note = The residue in this position is DNA
  modified_base 1..3: mod_base = OTHER; note = The residue in this position has a 3-phosphorothioate.
  modified_base 51..53: mod_base = OTHER; note = The residue in this position has a 3-phosphorothioate.

SEQ ID NO: 52 - RNA, 54 nt, synthetic construct; identical to SEQ ID NO: 25
  source 1..54: mol_type = other RNA
  modified_base 1..3: mod_base = OTHER; note = The residue in this position is a 2-O-methylated ribonucleotide.
  modified_base 52..54: mod_base = OTHER; note = The residue in this position is a 2-O-methylated ribonucleotide.
  modified_base 24..40: mod_base = OTHER; note = The residue in this position is DNA
  modified_base 1..3: mod_base = OTHER; note = The residue in this position has a 3-phosphorothioate.
  modified_base 41..54: mod_base = OTHER; note = The residue in this position has a 3-phosphorothioate.

SEQ ID NO: 53 - RNA, 54 nt, synthetic construct; identical to SEQ ID NO: 25
  source 1..54: mol_type = other RNA
  modified_base 1..3: mod_base = OTHER; note = The residue in this position is a 2-O-methylated ribonucleotide.
  modified_base 41..54: mod_base = OTHER; note = The residue in this position is a 2-O-methylated ribonucleotide.
  modified_base 1..3: mod_base = OTHER; note = The residue in this position has a 3-phosphorothioate.
  modified_base 51..53: mod_base = OTHER; note = The residue in this position has a 3-phosphorothioate.
  modified_base 24..40: mod_base = OTHER; note = The residue in this position is DNA
  modified_base 24: mod_base = OTHER; note = The residue in this position has a 2-Fluoro modification.
  modified_base 26: mod_base = OTHER; note = The residue in this position has a 2-Fluoro modification.
  modified_base 28: mod_base = OTHER; note = The residue in this position has a 2-Fluoro modification.
  modified_base 30: mod_base = OTHER; note = The residue in this position has a 2-Fluoro modification.
  modified_base 32: mod_base = OTHER; note = The residue in this position has a 2-Fluoro modification.
  modified_base 34: mod_base = OTHER; note = The residue in this position is Uracil
  modified_base 34: mod_base = OTHER; note = The residue in this position has a 2-Fluoro modification.
  modified_base 36: mod_base = OTHER; note = The residue in this position has a 2-Fluoro modification.
  modified_base 38: mod_base = OTHER; note = The residue in this position has a 2-Fluoro modification.
  modified_base 40: mod_base = OTHER; note = The residue in this position is Uracil
  modified_base 40: mod_base = OTHER; note = The residue in this position has a 2-Fluoro modification.
  modified_base 42: mod_base = OTHER; note = The residue in this position has a 2-Fluoro modification.
  modified_base 44: mod_base = OTHER; note = The residue in this position has a 2-Fluoro modification.
  modified_base 46: mod_base = OTHER; note = The residue in this position has a 2-Fluoro modification.
  modified_base 48: mod_base = OTHER; note = The residue in this position has a 2-Fluoro modification.
  modified_base 50: mod_base = OTHER; note = The residue in this position has a 2-Fluoro modification.

SEQ ID NO: 54 - RNA, 54 nt, synthetic construct; identical to SEQ ID NO: 25
  source 1..54: mol_type = other RNA
  modified_base 1..3: mod_base = OTHER; note = The residue in this position is a 2-O-methylated ribonucleotide.
  modified_base 44..54: mod_base = OTHER; note = The residue in this position is a 2-O-methylated ribonucleotide.
  modified_base 24..43: mod_base = OTHER; note = The residue in this position is DNA
  modified_base 1..3: mod_base = OTHER; note = This residue has a 3-phosphorothioate.
  modified_base 51..53: mod_base = OTHER; note = This residue has a 3-phosphorothioate.

SEQ ID NO: 55 - RNA, 54 nt, synthetic construct; identical to SEQ ID NO: 25
  source 1..54: mol_type = other RNA
  modified_base 1..3: mod_base = OTHER; note = The residue in this position is a 2-O-methylated ribonucleotide.
  modified_base 52..54: mod_base = OTHER; note = The residue in this position is a 2-O-methylated ribonucleotide.
  modified_base 24..43: mod_base = OTHER; note = The residue at this position is DNA
  modified_base 44: mod_base = OTHER; note = This residue has a 3-phosphorothioate.
  modified_base 46: mod_base = OTHER; note = This residue has a 3-phosphorothioate.
  modified_base 50..52: mod_base = OTHER; note = This residue has a 3-phosphorothioate.
  modified_base 1..3: mod_base = OTHER; note = This residue has a 3-phosphorothioate.
  modified_base 48: mod_base = OTHER; note = This residue has a 3-phosphorothioate.

SEQ ID NO: 56 - RNA, 54 nt, synthetic construct; identical to SEQ ID NO: 25
  source 1..54: mol_type = other RNA
  modified_base 1..3: mod_base = OTHER; note = The residue in this position is a 2-O-methylated ribonucleotide.
  modified_base 52..54: mod_base = OTHER; note = The residue in this position is a 2-O-methylated ribonucleotide.
  modified_base 24..40: mod_base = OTHER; note = The residue in this position is DNA
  modified_base 1..3: mod_base = OTHER; note = This residue has a 3-phosphorothioate.
  modified_base 43: mod_base = OTHER; note = The residue in this position is LDNA.
  modified_base 46: mod_base = OTHER; note = The residue in this position is LDNA
  modified_base 49: mod_base = OTHER; note = The residue in this position is LDNA.
  modified_base 52..54: mod_base = OTHER; note = This residue has a 3-phosphorothioate.

SEQ ID NO: 57 - RNA, 54 nt, synthetic construct; identical to SEQ ID NO: 25
  source 1..54: mol_type = other RNA
  modified_base 1..3: mod_base = OTHER; note = The residue in this position is a 2-O-methylated ribonucleotide.
  modified_base 24..54: mod_base = OTHER; note = The residue in this position is DNA
  modified_base 1..3: mod_base = OTHER; note = This residue has a 3-phosphorothioate.

SEQ ID NO: 58 - RNA, 54 nt, synthetic construct; identical to SEQ ID NO: 25
  source 1..54: mol_type = other RNA
  modified_base 1..3: mod_base = OTHER; note = The residue in this position is a 2-O-methylated ribonucleotide.
  modified_base 52..54: mod_base = OTHER; note = This residue has a 3-phosphorothioate.
  modified_base 24..51: mod_base = OTHER; note = The residue at this position is DNA
  modified_base 51..53: mod_base = OTHER; note = The residue in this position is a 2-O-methylated ribonucleotide.
  modified_base 1..3: mod_base = OTHER; note = This residue has a 3-phosphorothioate.

SEQ ID NO: 59 - RNA, 54 nt, synthetic construct; identical to SEQ ID NO: 25
  source 1..54: mol_type = other RNA
  modified_base 1..3: mod_base = OTHER; note = The residue in this position is a 2-O-methylated ribonucleotide.
  modified_base 51..52: mod_base = OTHER; note = The residue in this position is a 2-O-methylated ribonucleotide.
  modified_base 24..51: mod_base = OTHER; note = The residue in this position is DNA
  modified_base 51..53: mod_base = OTHER; note = This residue has a 3-phosphorothioate.
  modified_base 54: mod_base = OTHER; note = The residue in this position is DNA

SEQ ID NO: 60 - DNA, 31 bp, synthetic construct; identical to SEQ ID NO: 43
  source 1..31: mol_type = other DNA

SEQ ID NO: 61 - RNA, 31 nt, synthetic construct; identical to SEQ ID NO: 42
  source 1..31: mol_type = other RNA
  modified_base 1..17: mod_base = OTHER; note = The residue in this position is DNA
  modified_base 29..31: mod_base = OTHER; note = The residue in this position is a 2-O-methylated ribonucleotide.
  modified_base 28..30: mod_base = OTHER; note = This residue has a 3-phosphorothioate.

SEQ ID NO: 62 - RNA, 31 nt, synthetic construct; identical to SEQ ID NO: 42
  source 1..31: mol_type = other RNA
  modified_base 1..25: mod_base = OTHER; note = The residue in this position is DNA
  modified_base 29..31: mod_base = OTHER; note = The residue in this position is a 2-O-methylated ribonucleotide.
  modified_base 28..30: mod_base = OTHER; note = This residue has a 3-phosphorothioate.

SEQ ID NO: 63 - DNA, 31 bp, synthetic construct; identical to SEQ ID NO: 43
  source 1..31: mol_type = other DNA
  modified_base 27..30: mod_base = OTHER; note = This residue has a 3-phosphorothioate.

SEQ ID NO: 64 - DNA, 31 bp, synthetic construct; identical to SEQ ID NO: 43
  source 1..31: mol_type = other DNA
  modified_base 25..30: mod_base = OTHER; note = This residue has a 3-phosphorothioate.

SEQ ID NO: 65 - DNA, 31 bp, synthetic construct; identical to SEQ ID NO: 43
  source 1..31: mol_type = other DNA
  modified_base 23..30: mod_base = OTHER; note = This residue has a 3-phosphorothioate.

SEQ ID NO: 66 - DNA, 31 bp, synthetic construct; identical to SEQ ID NO: 43
  source 1..31: mol_type = other DNA
  modified_base 21..30: mod_base = OTHER; note = This residue has a 3-phosphorothioate.

SEQ ID NO: 67 - RNA, 47 nt, synthetic construct
  source 1..47: mol_type = other RNA
  modified_base 1..3: mod_base = OTHER; note = The residue in this position is a 2-O-methylated ribonucleotide.
  modified_base 45..47: mod_base = OTHER; note = The residue in this position is a 2-O-methylated ribonucleotide.
  modified_base 1..3: mod_base = OTHER; note = This residue has a 3-phosphorothioate.
  modified_base 44..46: mod_base = OTHER; note = This residue has a 3-phosphorothioate.
gcacatgaggatcacccatgtgcatgtagacgccaaggccccccacc

SEQ ID NO: 68 - RNA, 47 nt, synthetic construct; identical to SEQ ID NO: 67
  source 1..47: mol_type = other RNA
  modified_base 1..3: mod_base = OTHER; note = The residue in this position is a 2-O-methylated ribonucleotide.
  modified_base 45..47: mod_base = OTHER; note = The residue in this position is a 2-O-methylated ribonucleotide.
  modified_base 24..29: mod_base = OTHER; note = The residue in this position is DNA
  modified_base 1..3: mod_base = OTHER; note = This residue has a 3-phosphorothioate.
  modified_base 44..46: mod_base = OTHER; note = This residue has a 3-phosphorothioate.

SEQ ID NO: 69 - RNA, 47 nt, synthetic construct; identical to SEQ ID NO: 67
  source 1..47: mol_type = other RNA
  modified_base 1..3: mod_base = OTHER; note = The residue in this position is a 2-O-methylated ribonucleotide.
  modified_base 45..47: mod_base = OTHER; note = The residue in this position is a 2-O-methylated ribonucleotide.
  modified_base 24..31: mod_base = OTHER; note = The residue at this position is DNA
  modified_base 1..3: mod_base = OTHER; note = This residue has a 3-phosphorothioate.
  modified_base 44..46: mod_base = OTHER; note = This residue has a 3-phosphorothioate.

SEQ ID NO: 70 - RNA, 47 nt, synthetic construct; identical to SEQ ID NO: 67
  source 1..47: mol_type = other RNA
  modified_base 1..3: mod_base = OTHER; note = The residue in this position is a 2-O-methylated ribonucleotide.
  modified_base 45..47: mod_base = OTHER; note = The residue in this position is a 2-O-methylated ribonucleotide.
  modified_base 24..35: mod_base = OTHER; note = The residue in the position is DNA
  modified_base 1..3: mod_base = OTHER; note = This residue has a 3-phosphorothioate.
  modified_base 44..46: mod_base = OTHER; note = This residue has a 3-phosphorothioate.

SEQ ID NO: 71 - RNA, 47 nt, synthetic construct; identical to SEQ ID NO: 67
  source 1..47: mol_type = other RNA
  modified_base 1..3: mod_base = OTHER; note = The residue in this position is a 2-O-methylated ribonucleotide.
  modified_base 45..47: mod_base = OTHER; note = The residue in this position is a 2-O-methylated ribonucleotide.
  modified_base 24..37: mod_base = OTHER; note = The residue in this position is DNA
  modified_base 1..3: mod_base = OTHER; note = This residue has a 3-phosphorothioate.
  modified_base 44..46: mod_base = OTHER; note = This residue has a 3-phosphorothioate.

SEQ ID NO: 72 - RNA, 47 nt, synthetic construct; identical to SEQ ID NO: 67
  source 1..47: mol_type = other RNA
  modified_base 1..3: mod_base = OTHER; note = The residue in this position is a 2-O-methylated ribonucleotide.
  modified_base 45..47: mod_base = OTHER; note = The residue in this position is a 2-O-methylated ribonucleotide.
  modified_base 24..39: mod_base = OTHER; note = The residue in this position is DNA
  modified_base 1..3: mod_base = OTHER; note = This residue has a 3-phosphorothioate.
  modified_base 44..46: mod_base = OTHER; note = This residue has a 3-phosphorothioate.

SEQ ID NO: 73 - RNA, 47 nt, synthetic construct; identical to SEQ ID NO: 67
  source 1..47: mol_type = other RNA
  modified_base 1..3: mod_base = OTHER; note = The residue in this position is a 2-O-methylated ribonucleotide.
  modified_base 45..47: mod_base = OTHER; note = The residue in this position is a 2-O-methylated ribonucleotide.
  modified_base 24..41: mod_base = OTHER; note = The residue in this position is DNA
  modified_base 1..3: mod_base = OTHER; note = This residue has a 3-phosphorothioate.
  modified_base 44..46: mod_base = OTHER; note = This residue has a 3-phosphorothioate.

SEQ ID NO: 74 - RNA, 47 nt, synthetic construct; identical to SEQ ID NO: 67
  source 1..47: mol_type = other RNA
  modified_base 1..3: mod_base = OTHER; note = The residue in this position is a 2-O-methylated ribonucleotide.
  modified_base 45..47: mod_base = OTHER; note = The residue in this position is a 2-O-methylated ribonucleotide.
  modified_base 24..43: mod_base = OTHER; note = The residue in this position is DNA
  modified_base 1..3: mod_base = OTHER; note = This residue has a 3-phosphorothioate.
  modified_base 44..46: mod_base = OTHER; note = This residue has a 3-phosphorothioate.

SEQ ID NO: 75 - RNA, 52 nt, synthetic construct
  source 1..52: mol_type = other RNA
  modified_base 1..3: mod_base = OTHER; note = The residue in this position is a 2-O-methylated ribonucleotide.
  modified_base 50..52: mod_base = OTHER; note = The residue in this position is a 2-O-methylated ribonucleotide.
  modified_base 1..3: mod_base = OTHER; note = This residue has a 3-phosphorothioate.
  modified_base 49..51: mod_base = OTHER; note = This residue has a 3-phosphorothioate.
gcacatgaggatcacccatgtgcgccagtatccctggccttcagacatccct

SEQ ID NO: 76 - RNA, 52 nt, synthetic construct; identical to SEQ ID NO: 75
  source 1..52: mol_type = other RNA
  modified_base 1..3: mod_base = OTHER; note = The residue in this position is a 2-O-methylated ribonucleotide.
  modified_base 50..52: mod_base = OTHER; note = The residue in this position is a 2-O-methylated ribonucleotide.
  modified_base 24..39: mod_base = OTHER; note = The residue in this position is DNA
  modified_base 1..3: mod_base = OTHER; note = This residue has a 3-phosphorothioate.
  modified_base 49..51: mod_base = OTHER; note = This residue has a 3-phosphorothioate.

SEQ ID NO: 77 - RNA, 50 nt, synthetic construct
  source 1..50: mol_type = other RNA
  modified_base 1..3: mod_base = OTHER; note = The residue in this position is a 2-O-methylated ribonucleotide.
  modified_base 48..50: mod_base = OTHER; note = The residue in this position is a 2-O-methylated ribonucleotide.
  modified_base 24..40: mod_base = OTHER; note = The residue in this position is DNA
  modified_base 1..3: mod_base = OTHER; note = This residue has a 3-phosphorothioate.
  modified_base 47..49: mod_base = OTHER; note = This residue has a 3-phosphorothioate.
gcacatgaggatcacccatgtgcagacttctctacaggagtcaggtgcac

SEQ ID NO: 78 - RNA, 56 nt, synthetic construct
  source 1..56: mol_type = other RNA
  modified_base 1..3: mod_base = OTHER; note = The residue in this position is a 2-O-methylated ribonucleotide.
  modified_base 54..56: mod_base = OTHER; note = The residue in this position is a 2-O-methylated ribonucleotide.
  modified_base 24..43: mod_base = OTHER; note = The residue in this position is DNA
  modified_base 1..3: mod_base = OTHER; note = This residue has a 3-phosphorothioate.
  modified_base 53..56: mod_base = OTHER; note = This residue has a 3-phosphorothioate.
gcacatgaggatcacccatgtgctgtctgaagccatccatgcttcctcctgaaaat

SEQ ID NO: 79 - DNA, 24 bp, synthetic construct
  source 1..24: mol_type = other DNA
atgtagacgccaaggccccccacc

SEQ ID NO: 80 - DNA, 29 bp, synthetic construct
  source 1..29: mol_type = other DNA
gccagtatccctggccttcagacatccct

SEQ ID NO: 81 - RNA, 96 nt, synthetic construct
  source 1..96: mol_type = other RNA
  modified_base 2..3: mod_base = OTHER; note = The residue in this position is a 2-O-methylated ribonucleotide.
  modified_base 94..96: mod_base = OTHER; note = The residue in this position is a 2-O-methylated ribonucleotide.
  modified_base 24..83: mod_base = OTHER; note = The residue in this position is DNA
  modified_base 1..3: mod_base = OTHER; note = This residue has a 3-phosphorothioate.
  modified_base 93..95: mod_base = OTHER; note = This residue has a 3-phosphorothioate.
gcacatgaggatcacccatgtgcgatggagccagagaggaattgttataacttcgtatagcatacattatacgaagttatcccagaacctgagttt

SEQ ID NO: 82 - RNA, 96 nt, synthetic construct
  source 1..96: mol_type = other RNA
  modified_base 2..3: mod_base = OTHER; note = The residue in this position is a 2-O-methylated ribonucleotide.
  modified_base 94..96: mod_base = OTHER; note = The residue in this position is a 2-O-methylated ribonucleotide.
  modified_base 24..83: mod_base = OTHER; note = The residue in this position is DNA
  modified_base 1..3: mod_base = OTHER; note = This residue has a 3-phosphorothioate.
  modified_base 93..95: mod_base = OTHER; note = This residue has a 3-phosphorothioate.
gcacatgaggatcacccatgtgcgcagctcaggttctgggataacttcgtataatgtatgctatacgaagttataacaattcctctctggctcttt

SEQ ID NO: 83 - RNA, 120 nt, synthetic construct
  source 1..120: mol_type = other RNA
  modified_base 1..3: mod_base = OTHER; note = The residue in this position is a 2-O-methylated ribonucleotide.
  modified_base 118..120: mod_base = OTHER; note = The residue in this position is a 2-O-methylated ribonucleotide.
  modified_base 24..107: mod_base = OTHER; note = The residue in this position is DNA
  modified_base 1..3: mod_base = OTHER; note = This residue has a 3-phosphorothioate.
  modified_base 117..119: mod_base = OTHER; note = This residue has a 3-phosphorothioate.
gcacatgaggatcacccatgtgcgctgaacttgtggccgtttacgtcgccgtccagctcgaccaggatgggcaccaccccggtgaacagctcctcgcccttgctcaccgtgctcagtctg

SEQ ID NO: 84 - RNA, 120 nt, synthetic construct
  source 1..120: mol_type = other RNA
  modified_base 1..3: mod_base = OTHER; note = The residue in this position is a 2-O-methylated ribonucleotide.
  modified_base 118..120: mod_base = OTHER; note = The residue in this position is a 2-O-methylated ribonucleotide.
  modified_base 24..106: mod_base = OTHER; note = The residue in this position is DNA
  modified_base 1..3: mod_base = OTHER; note = This residue has a 3-phosphorothioate.
  modified_base 117..119: mod_base = OTHER; note = This residue has a 3-phosphorothioate.
gcacatgaggatcacccatgtgcagctggacggcgacgtaaacggccacaagttcagcgtgtccggcgagggcgagggcgatgccacctacggcaagctgaccctgccgggatactggtt

SEQ ID NO: 85 - RNA, 53 nt, synthetic construct
  source 1..53: mol_type = other RNA
  modified_base 1..3: mod_base = OTHER; note = The residue in this position is a 2-O-methylated ribonucleotide.
  modified_base 51..53: mod_base = OTHER; note = The residue in this position is a 2-O-methylated ribonucleotide.
  modified_base 24..44: mod_base = OTHER; note = The residue in this position is DNA
  modified_base 1..3: mod_base = OTHER; note = This residue has a 3-phosphorothioate.
  modified_base 50..52: mod_base = OTHER; note = This residue has a 3-phosphorothioate.
gcacatgaggatcacccatgtgctttgtctcgccaaaggtctccggacacgct

SEQ ID NO: 86 - DNA, 30 bp, synthetic construct
  source 1..30: mol_type = other DNA
tttgtctcgccaaaggtctccggacacgct

SEQ ID NO: 87 - DNA, 42 bp, synthetic construct
  source 1..42: mol_type = other DNA
ctacacgacgctcttccgatctgatggatgtggcgcaggtag

SEQ ID NO: 88 - DNA, 41 bp, synthetic construct
  source 1..41: mol_type = other DNA
agacgtgtgctcttccgatctaggcgtatcatttcgcggat

SEQ ID NO: 89 - DNA, 41 bp, synthetic construct
  source 1..41: mol_type = other DNA
ctacacgacgctcttccgatctgaccaaagcgccgatggat

SEQ ID NO: 90 - DNA, 41 bp, synthetic construct
  source 1..41: mol_type = other DNA
agacgtgtgctcttccgatctggtgaaagcggaagtagggc

SEQ ID NO: 91 - DNA, 46 bp, synthetic construct
  source 1..46: mol_type = other DNA
ctacacgacgctcttccgatctgtgcgtatggaatagcccatgatc

SEQ ID NO: 92 - DNA, 42 bp, synthetic construct
  source 1..42: mol_type = other DNA
agacgtgtgctcttccgatctacgttgagctgtgcagagaag

SEQ ID NO: 93 - DNA, 47 bp, synthetic construct
  source 1..47: mol_type = other DNA
ctacacgacgctcttccgatctagtaagccaaaaaccaacatgaagc

SEQ ID NO: 94 - DNA, 42 bp, synthetic construct
  source 1..42: mol_type = other DNA
agacgtgtgctcttccgatctctgtactcatccatgggcctg

SEQ ID NO: 95 - DNA, 54 bp, synthetic construct
  source 1..54: mol_type = other DNA
acatgaagcctacacgacgctcttccgatcttcgctccgaaggtaaaagaaatc

SEQ ID NO: 96 - DNA, 44 bp, synthetic construct
  source 1..44: mol_type = other DNA
agacgtgtgctcttccgatctcaggcaaagctgagcaaaagtag

SEQ ID NO: 97 - DNA, 48 bp, synthetic construct
  source 1..48: mol_type = other DNA
ctacacgacgctcttccgatctttgcttacatttgcttctgacacaac

SEQ ID NO: 98 - DNA, 40 bp, synthetic construct
  source 1..40: mol_type = other DNA
agacgtgtgctcttccgatcttctgtctccacatgcccag

SEQ ID NO: 99 - DNA, 42 bp, synthetic construct
  source 1..42: mol_type = other DNA
ctacacgacgctcttccgatctccaggatcagtgaaacgcac

SEQ ID NO: 100 - DNA, 41 bp, synthetic construct
  source 1..41: mol_type = other DNA
agacgtgtgctcttccgatctcttgccagaacctctaaggt

SEQ ID NO: 101 - DNA, 22 bp, synthetic construct
  source 1..22: mol_type = other DNA
cctggcctgggtcaatccttgg

SEQ ID NO: 102 - DNA, 19 bp, synthetic construct
  source 1..19: mol_type = other DNA
cggcgacgtaaacggccac

SEQ ID NO: 103 - DNA, 21 bp, synthetic construct
  source 1..21: mol_type = other DNA
caccaccccggtgaacagctc

SEQ ID NO: 104 - DNA, 22 bp, synthetic construct
  source 1..22: mol_type = other DNA
gcatgcatttgtaggcttgatg

SEQ ID NO: 105 - DNA, 20 bp, synthetic construct
  source 1..20: mol_type = other DNA
cagccaaacttgtcaaccag

SEQ ID NO: 106 - RNA, 31 nt, synthetic construct; identical to SEQ ID NO: 42
  source 1..31: mol_type = other RNA

SEQ ID NO: 107 - RNA, 31 nt, synthetic construct; identical to SEQ ID NO: 42
  source 1..31: mol_type = other RNA

SEQ ID NO: 108 - RNA, 31 nt, synthetic construct; identical to SEQ ID NO: 42
  source 1..31: mol_type = other RNA

SEQ ID NO: 109 - RNA, 31 nt, synthetic construct; identical to SEQ ID NO: 42
  source 1..31: mol_type = other RNA
  modified_base 28..30: mod_base = OTHER; note = This residue has a 3-phosphorothioate.
  modified_base 29..31: mod_base = OTHER; note = The residues at these positions are 2-O-methylated ribonucleotides

SEQ ID NO: 110 - RNA, 31 nt, synthetic construct; identical to SEQ ID NO: 42
  source 1..31: mol_type = other RNA
  modified_base 28..30: mod_base = OTHER; note = This residue has a 3-phosphorothioate.
  modified_base 29..31: mod_base = OTHER; note = The residues at these positions are 2-O-methylated ribonucleotides

SEQ ID NO: 111 - DNA, 30 bp, synthetic construct; identical to SEQ ID NO: 1
  source 1..30: mol_type = other DNA

SEQ ID NO: 112 - DNA, 30 bp, synthetic construct; identical to SEQ ID NO: 3
  source 1..30: mol_type = other DNA

SEQ ID NO: 113 - RNA, 31 nt, synthetic construct; identical to SEQ ID NO: 42
  source 1..31: mol_type = other RNA
  modified_base 1..11: mod_base = OTHER; note = The residue in this position is DNA
  modified_base 28..30: mod_base = OTHER; note = The residue at this position has a 3-phosphorothioate.

SEQ ID NO: 114 - RNA, 31 nt, synthetic construct; identical to SEQ ID NO: 42
  source 1..31: mol_type = other RNA
  modified_base 1..12: mod_base = OTHER; note = The residue in this position is DNA
  modified_base 28..30: mod_base = OTHER; note = The residue at this position has a 3-phosphorothioate.

SEQ ID NO: 115 - RNA, 31 nt, synthetic construct; identical to SEQ ID NO: 42
  source 1..31: mol_type = other RNA
  modified_base 1..13: mod_base = OTHER; note = The residue in this position is DNA
  modified_base 28..30: mod_base = OTHER; note = The residue at this position has a 3-phosphorothioate.

SEQ ID NO: 116 - skipped

SEQ ID NO: 117 - RNA, 31 nt, synthetic construct; identical to SEQ ID NO: 42
  source 1..31: mol_type = other RNA
  modified_base 1..15: mod_base = OTHER; note = The residue in this position is DNA
  modified_base 28..30: mod_base = OTHER; note = The residue at this position has a 3-phosphorothioate.

SEQ ID NO: 118 - RNA, 31 nt, synthetic construct; identical to SEQ ID NO: 42
  source 1..31: mol_type = other RNA
  modified_base 1..16: mod_base = OTHER; note = The residue in this position is DNA
  modified_base 28..30: mod_base = OTHER; note = The residue at this position has a 3-phosphorothioate.

SEQ ID NO: 119 - RNA, 31 nt, synthetic construct; identical to SEQ ID NO: 42
  source 1..31: mol_type = other RNA
  modified_base 1..17: mod_base = OTHER; note = The residue in this position is DNA
  modified_base 28..30: mod_base = OTHER; note = The residue at this position has a 3-phosphorothioate.

SEQ ID NO: 120 - RNA, 31 nt, synthetic construct; identical to SEQ ID NO: 42
  source 1..31: mol_type = other RNA
  modified_base 1..18: mod_base = OTHER; note = The residue in this position is DNA
  modified_base 28..30: mod_base = OTHER; note = The residue at this position has a 3-phosphorothioate.

SEQ ID NO: 121 - RNA, 31 nt, synthetic construct; identical to SEQ ID NO: 42
  source 1..31: mol_type = other RNA
  modified_base 1..19: mod_base = OTHER; note = The residue in this position is DNA
  modified_base 28..30: mod_base = OTHER; note = The residue at this position has a 3-phosphorothioate.

SEQ ID NO: 122 - RNA, 31 nt, synthetic construct; identical to SEQ ID NO: 42
  source 1..31: mol_type = other RNA
  modified_base 1..20: mod_base = OTHER; note = The residue in this position is DNA
  modified_base 28..30: mod_base = OTHER; note = The residue at this position has a 3-phosphorothioate.

SEQ ID NO: 123 - RNA, 31 nt, synthetic construct; identical to SEQ ID NO: 42
  source 1..31: mol_type = other RNA
  modified_base 1..21: mod_base = OTHER; note = The residue in this position is DNA
  modified_base 28..30: mod_base = OTHER; note = The residue at this position has a 3-phosphorothioate.

SEQ ID NO: 124 - RNA, 31 nt, synthetic construct; identical to SEQ ID NO: 42
  source 1..31: mol_type = other RNA
  modified_base 1..22: mod_base = OTHER; note = The residue in this position is DNA
  modified_base 28..30: mod_base = OTHER; note = The residue at this position has a 3-phosphorothioate.

SEQ ID NO: 125 - RNA, 31 nt, synthetic construct; identical to SEQ ID NO: 42
  source 1..31: mol_type = other RNA
  modified_base 1..23: mod_base = OTHER; note = The residue in this position is DNA
  modified_base 28..30: mod_base = OTHER; note = The residue at this position has a 3-phosphorothioate.

SEQ ID NO: 126 - RNA, 31 nt, synthetic construct; identical to SEQ ID NO: 42
  source 1..31: mol_type = other RNA
  modified_base 1..24: mod_base = OTHER; note = The residue in this position is DNA
  modified_base 28..30: mod_base = OTHER; note = The residue at this position has a 3-phosphorothioate.

SEQ ID NO: 127 - RNA, 31 nt, synthetic construct; identical to SEQ ID NO: 42
  source 1..31: mol_type = other RNA
  modified_base 1..25: mod_base = OTHER; note = The residue in this position is DNA
  modified_base 28..30: mod_base = OTHER; note = The residue at this position has a 3-phosphorothioate.

SEQ ID NO: 128 - RNA, 31 nt, synthetic construct; identical to SEQ ID NO: 42
  source 1..31: mol_type = other RNA
  modified_base 1..26: mod_base = OTHER; note = The residue in this position is DNA
  modified_base 28..30: mod_base = OTHER; note = The residue at this position has a 3-phosphorothioate.

SEQ ID NO: 129 - RNA, 31 nt, synthetic construct; identical to SEQ ID NO: 42
  source 1..31: mol_type = other RNA
  modified_base 1..27: mod_base = OTHER; note = The residue in this position is DNA
  modified_base 28..30: mod_base = OTHER; note = The residue at this position has a 3-phosphorothioate.

SEQ ID NO: 130 - RNA, 31 nt, synthetic construct; identical to SEQ ID NO: 42
  source 1..31: mol_type = other RNA
  modified_base 1..28: mod_base = OTHER; note = The residue in this position is DNA
  modified_base 28..30: mod_base = OTHER; note = The residue at this position has a 3-phosphorothioate.

SEQ ID NO: 131 - DNA, 16 bp, synthetic construct
  source 1..16: mol_type = other DNA
gcagcacctggatcgc

SEQ ID NO: 132 - DNA, 16 bp, synthetic construct
  source 1..16: mol_type = other DNA
gcagcatctggatcgc

SEQ ID NO: 133 - RNA, 31 nt, synthetic construct; identical to SEQ ID NO: 42
  source 1..31: mol_type = other RNA
  modified_base 1..17: mod_base = OTHER; note = The residue in this position is DNA
  modified_base 28..30: mod_base = OTHER; note = The residue at this position has a 3-phosphorothioate.

SEQ ID NO: 134 - RNA, 31 nt, synthetic construct; identical to SEQ ID NO: 42
  source 1..31: mol_type = other RNA
  modified_base 1..18: mod_base = OTHER; note = The residue in this position is DNA
  modified_base 28..30: mod_base = OTHER; note = The residue at this position has a 3-phosphorothioate.

SEQ ID NO: 135 - RNA, 31 nt, synthetic construct; identical to SEQ ID NO: 42
  source 1..31: mol_type = other RNA
  modified_base 1..19: mod_base = OTHER; note = The residue in this position is DNA
  modified_base 28..30: mod_base = OTHER; note = The residue at this position has a 3-phosphorothioate.

SEQ ID NO: 136 - RNA, 31 nt, synthetic construct; identical to SEQ ID NO: 42
  source 1..31: mol_type = other RNA
  modified_base 1..20: mod_base = OTHER; note = The residue in this position is DNA
  modified_base 28..30: mod_base = OTHER; note = The residue at this position has a 3-phosphorothioate.

SEQ ID NO: 137 - RNA, 31 nt, synthetic construct; identical to SEQ ID NO: 42
  source 1..31: mol_type = other RNA
  modified_base 1..21: mod_base = OTHER; note = The residue in this position is DNA
  modified_base 28..30: mod_base = OTHER; note = The residue at this position has a 3-phosphorothioate.

SEQ ID NO: 138 - DNA, 31 bp, synthetic construct; identical to SEQ ID NO: 43
  source 1..31: mol_type = other DNA
  modified_base 14: mod_base = OTHER; note = The residue in this position is Uracil

SEQ ID NO: 139 - RNA, 31 nt, synthetic construct; identical to SEQ ID NO: 42
  source 1..31: mol_type = other RNA
  modified_base 28..30: mod_base = OTHER; note = The residue at this position has a 3-phosphorothioate.

SEQ ID NO: 140 - RNA, 31 nt, synthetic construct; identical to SEQ ID NO: 42
  source 1..31: mol_type = other RNA
  modified_base 1..17: mod_base = OTHER; note = The residue in this position is DNA
  modified_base 28..30: mod_base = OTHER; note = The residue at this position has a 3-phosphorothioate.
  modified_base 14: mod_base = OTHER; note = The residue at this position is Uracil.

SEQ ID NO: 141 - RNA, 31 nt, synthetic construct; identical to SEQ ID NO: 42
  source 1..31: mol_type = other RNA
  modified_base 1..25: mod_base = OTHER; note = The residue in this position is DNA
  modified_base 28..30: mod_base = OTHER; note = The residue at this position has a 3-phosphorothioate.
  modified_base 14: mod_base = OTHER; note = The residue at this position is Uracil.

SEQ ID NO: 142 - DNA, 31 bp, synthetic construct; identical to SEQ ID NO: 43
  source 1..31: mol_type = other DNA
  modified_base 27..30: mod_base = OTHER; note = The residue at this position has a 3-phosphorothioate.
  modified_base 14: mod_base = OTHER; note = The residue at this position is Uracil

SEQ ID NO: 143 - DNA, 31 bp, synthetic construct; identical to SEQ ID NO: 43
  source 1..31: mol_type = other DNA
  modified_base 25..30: mod_base = OTHER; note = The residue at this position has a 3-phosphorothioate.
  modified_base 14: mod_base = OTHER; note = The residue at this position is Uracil.

SEQ ID NO: 144 - DNA, 31 bp, synthetic construct; identical to SEQ ID NO: 43
  source 1..31: mol_type = other DNA
  modified_base 23..30: mod_base = OTHER; note = The residue at this position has a 3-phosphorothioate.
  modified_base 14: mod_base = OTHER; note = The residue in this position is Uracil.

SEQ ID NO: 145 - DNA, 31 bp, synthetic construct; identical to SEQ ID NO: 43
  source 1..31: mol_type = other DNA
  modified_base 21..30: mod_base = OTHER; note = The residue at this position has a 3-phosphorothioate.
  modified_base 14: mod_base = OTHER; note = The residue at this position is Uracil.

SEQ ID NO: 146 - RNA, 133 nt, synthetic construct
  source 1..133: mol_type = other RNA
caccttcagcttggcggtctgttttagagctagaaatagcaagttaaaataaggctagtccgttatcaacttgaaaaagtgggaccgagtcggtccaaaaccgtcagaggacacggttaaaaagtcctctttt

SEQ ID NO: 147 - RNA, 31 nt, synthetic construct
  source 1..31: mol_type = other RNA
  modified_base 28..30: mod_base = OTHER; note = The residue at this position has a 3-phosphorothioate.
ggaaaagcgatcgtgttgctgcagaagggat

SEQ ID NO: 148 - RNA, 31 nt, synthetic construct; identical to SEQ ID NO: 147
  source 1..31: mol_type = other RNA
  modified_base 1..6: mod_base = OTHER; note = The residue in this position is DNA
  modified_base 28..30: mod_base = OTHER; note = The residue at this position has a 3-phosphorothioate.

SEQ ID NO: 149 - RNA, 31 nt, synthetic construct; identical to SEQ ID NO: 147
  source 1..31: mol_type = other RNA
  modified_base 1..11: mod_base = OTHER; note = The residue in this position is DNA
  modified_base 28..30: mod_base = OTHER; note = The residue at this position has a 3-phosphorothioate.

SEQ ID NO: 150 - RNA, 31 nt, synthetic construct; identical to SEQ ID NO: 147
  source 1..31: mol_type = other RNA
  modified_base 1..12: mod_base = OTHER; note = The residue in this position is DNA
  modified_base 28..30: mod_base = OTHER; note = The residue at this position has a 3-phosphorothioate.

SEQ ID NO: 151 - RNA, 31 nt, synthetic construct; identical to SEQ ID NO: 147
  source 1..31: mol_type = other RNA
  modified_base 1..13: mod_base = OTHER; note = The residue in this position is DNA
  modified_base 28..30: mod_base = OTHER; note = The residue at this position has a 3-phosphorothioate.

SEQ ID NO: 152 - RNA, 31 nt, synthetic construct; identical to SEQ ID NO: 147
  source 1..31: mol_type = other RNA
  modified_base 1..14: mod_base = OTHER; note = The residue in this position is DNA
  modified_base 28..30: mod_base = OTHER; note = The residue at this position has a 3-phosphorothioate.

SEQ ID NO: 153 - RNA, 31 nt, synthetic construct; identical to SEQ ID NO: 147
  source 1..31: mol_type = other RNA
  modified_base 1..14: mod_base = OTHER; note = The residue in this position is DNA
  modified_base 28..30: mod_base = OTHER; note = The residue at this position has a 3-phosphorothioate.

SEQ ID NO: 154 - RNA, 31 nt, synthetic construct; identical to SEQ ID NO: 42
  source 1..31: mol_type = other RNA
  modified_base 1..16: mod_base = OTHER; note = The residue in this position is DNA
  modified_base 28..30: mod_base = OTHER; note = The residue at this position has a 3-phosphorothioate.

SEQ ID NO: 155 - RNA, 31 nt, synthetic construct; identical to SEQ ID NO: 42
  source 1..31: mol_type = other RNA
  modified_base 1..17: mod_base = OTHER; note = The residue in this position is DNA
  modified_base 28..30: mod_base = OTHER; note = The residue at this position has a 3-phosphorothioate.

SEQ ID NO: 156 - RNA, 31 nt, synthetic construct; identical to SEQ ID NO: 42
  source 1..31: mol_type = other RNA
  modified_base 1..18: mod_base = OTHER; note = The residue in this position is DNA
  modified_base 28..30: mod_base = OTHER; note = The residue at this position has a 3-phosphorothioate.

SEQ ID NO: 157 - RNA, 31 nt, synthetic construct; identical to SEQ ID NO: 42
  source 1..31: mol_type = other RNA
  modified_base 1..19: mod_base = OTHER; note = The residue in this position is DNA
  modified_base 28..30: mod_base = OTHER; note = The residue at this position has a 3-phosphorothioate.

SEQ ID NO: 158 - RNA, 31 nt, synthetic construct; identical to SEQ ID NO: 42
  source 1..31: mol_type = other RNA
  modified_base 1..20: mod_base = OTHER; note = The residue in this position is DNA
  modified_base 28..30: mod_base = OTHER; note = The residue at this position has a 3-phosphorothioate.

SEQ ID NO: 159 - RNA, 31 nt, synthetic construct; identical to SEQ ID NO: 42
  source 1..31: mol_type = other RNA
  modified_base 1..21: mod_base = OTHER; note = The residue in this position is DNA
  modified_base 28..30: mod_base = OTHER; note = The residue at this position has a 3-phosphorothioate.

SEQ ID NO: 160 - RNA, 31 nt, synthetic construct; identical to SEQ ID NO: 42
  source 1..31: mol_type = other RNA
  modified_base 1..22: mod_base = OTHER; note = The residue in this position is DNA
  modified_base 28..30: mod_base = OTHER; note = The residue at this position has a 3-phosphorothioate.

SEQ ID NO: 161 - RNA, 31 nt, synthetic construct; identical to SEQ ID NO: 42
  source 1..31: mol_type = other RNA
  modified_base 1..23: mod_base = OTHER; note = The residue in this position is DNA
  modified_base 28..30: mod_base = OTHER; note = The residue at this position has a 3-phosphorothioate.

SEQ ID NO: 162 - RNA, 31 nt, synthetic construct; identical to SEQ ID NO: 42
  source 1..31: mol_type = other RNA
  modified_base 1..24: mod_base = OTHER; note = The residue in this position is DNA
  modified_base 28..30: mod_base = OTHER; note = The residue at this position has a 3-phosphorothioate.

SEQ ID NO: 163 - RNA, 31 nt, synthetic construct; identical to SEQ ID NO: 42
  source 1..31: mol_type = other RNA
  modified_base 1..25: mod_base = OTHER; note = The residue in this position is DNA
  modified_base 28..30: mod_base = OTHER; note = The residue at this position has a 3-phosphorothioate.

SEQ ID NO: 164 - RNA, 31 nt, synthetic construct; identical to SEQ ID NO: 42
  source 1..31: mol_type = other RNA
  modified_base 1..26: mod_base = OTHER; note = The residue in this position is DNA
  modified_base 28..30: mod_base = OTHER; note = The residue at this position has a 3-phosphorothioate.

SEQ ID NO: 165 - RNA, 31 nt, synthetic construct; identical to SEQ ID NO: 42
  source 1..31: mol_type = other RNA
  modified_base 1..27: mod_base = OTHER; note = The residue in this position is DNA
  modified_base 28..30: mod_base = OTHER; note = The residue at this position has a 3-phosphorothioate.

SEQ ID NO: 166 - RNA, 31 nt, synthetic construct; identical to SEQ ID NO: 42
  source 1..31: mol_type = other RNA
  modified_base 1..28: mod_base = OTHER; note = The residue in this position is DNA
  modified_base 28..30: mod_base = OTHER; note = The residue at this position has a 3-phosphorothioate.

SEQ ID NO: 167 - RNA, 14 nt, synthetic construct
  source 1..14: mol_type = other RNA
  modified_base 11..13: mod_base = OTHER; note = The residue at this position has a 3-phosphorothioate.
gctgcagaagggat

SEQ ID NO: 168 - RNA, 14 nt, synthetic construct
  source 1..14: mol_type = other RNA
  modified_base 1: mod_base = OTHER; note = The residue in this position is DNA
  modified_base 11..13: mod_base = OTHER; note = The residue at this position has a 3-phosphorothioate.
gctgcagaagggat

SEQ ID NO: 169 - RNA, 14 nt, synthetic construct
  source 1..14: mol_type = other RNA
  modified_base 1..2: mod_base = OTHER; note = The residue in this position is DNA
  modified_base 11..13: mod_base = OTHER; note = The residue at this position has a 3-phosphorothioate.
gctgcagaagggat

SEQ ID NO: 170 - RNA, 14 nt, synthetic construct
  source 1..14: mol_type = other RNA
  modified_base 1..3: mod_base = OTHER; note = The residue in this position is DNA
  modified_base 11..13: mod_base = OTHER; note = The residue at this position has a 3-phosphorothioate.
gctgcagaagggat

SEQ ID NO: 171 - RNA, 14 nt, synthetic construct
  source 1..14: mol_type = other RNA
  modified_base 1..4: mod_base = OTHER; note = The residue in this position is DNA
  modified_base 11..13: mod_base = OTHER; note = The residue at this position has a 3-phosphorothioate.
gctgcagaagggat

SEQ ID NO: 172 - DNA, 68 bp, synthetic construct
  source 1..68: mol_type = other DNA
caggtgctgacgtaggtagtgcttgagaccgccagaagctcggaaaagcgatcgtgatgctgcagaag

SEQ ID NO: 173 - DNA, 68 bp, synthetic construct
  source 1..68: mol_type = other DNA
caggtgctgacgtaggtagtgcttgagaccgccagaagctcggaaaagcgatccaggtgctgcagaag

SEQ ID NO: 174 - DNA, 15 bp, synthetic construct
  source 1..15: mol_type = other DNA
caggtgctgcagaag

SEQ ID NO: 175 - DNA, 25 bp, synthetic construct
  source 1..25: mol_type = other DNA
aaaagcgatcgtgatgctgcagaag

SEQ ID NO: 176 - DNA, 12 bp, synthetic construct
  source 1..12: mol_type = other DNA
caggtgctgaag

SEQ ID NO: 177 - DNA, 12 bp, synthetic construct
  source 1..12: mol_type = other DNA
caggtgctgaag

SEQ ID NO: 178 - DNA, 68 bp, synthetic construct; identical to SEQ ID NO: 173
  source 1..68: mol_type = other DNA

SEQ ID NO: 179 - DNA, 15 bp, Synthetic construct
  source 1..15: mol_type = other DNA
caggtgctgcagaag

SEQ ID NO: 180 - DNA, 12 bp, synthetic construct
  source 1..12: mol_type = other DNA
caggtgctgaag

SEQ ID NO: 181 - DNA, 34 bp, synthetic construct
  source 1..34: mol_type = other DNA
catgtgcggaaaagcgatcgtgatgctgcagaag

SEQ ID NO: 182 - DNA, 81 bp, synthetic construct
  source 1..81: mol_type = other DNA
gccttggcggctacatgctgggaagtgccatgagcaggcccatcatacatttcggcagtgactatgaggaccgttactatc

SEQ ID NO: 183 - DNA, 81 bp, synthetic construct
  source 1..81: mol_type = other DNA
gccttggcgtctacatgctgggaagtgccatgagcaggcccatcatacatttcggcagtgactatgaggaccgttactatc

SEQ ID NO: 184 - DNA, 80 bp, synthetic construct
  source 1..80: mol_type = other DNA
gccttgcgtctacatgctgggaagtgccatgagcaggcccatcatacatttcggcagtgactatgaggaccgttactatc

SEQ ID NO: 185 - DNA, 81 bp, synthetic construct
  source 1..81: mol_type = other DNA
gccttggcggctacatgctgggaagtgtcatgagcaggcccatcatacatttcggcagtgactatgaggaccgttactatc

SEQ ID NO: 186 - DNA, 81 bp, synthetic construct
  source 1..81: mol_type = other DNA
ccttggccgtctacatgctgggaagtgccatgagcaggcccatcatacatttcggcagtgactatgaggaccgttactatc

SEQ ID NO: 187 - DNA, 81 bp, synthetic construct; identical to SEQ ID NO: 182
  source 1..81: mol_type = other DNA

SEQ ID NO: 188 - DNA, 81 bp, synthetic construct; identical to SEQ ID NO: 183
  source 1..81: mol_type = other DNA

SEQ ID NO: 189 - DNA, 81 bp, synthetic construct
  source 1..81: mol_type = other DNA
gccttggtgtctacatgctgggaagtgccatgagcaggcccatcatacatttcggcagtgactatgaggaccgttactatc

SEQ ID NO: 190 - DNA, 81 bp, synthetic construct
  source 1..81: mol_type = other DNA
gctttggcgtctacatgctgggaagtgccatgagcaggcccatcatacatttcggcagtgactatgaggaccgttactatc

SEQ ID NO: 191 - DNA, 80 bp, synthetic construct
  source 1..80: mol_type = other DNA
gcttggcgtctacatgctgggaagtgccatgagcaggcccatcatacatttcggcagtgactatgaggaccgttactatc

SEQ ID NO: 192 - RNA, 31 nt, synthetic construct; identical to SEQ ID NO: 42
  source 1..31: mol_type = other RNA
  modified_base 1..17: mod_base = OTHER; note = The residue in this position is DNA
  modified_base 20: mod_base = OTHER; note = The residue in this position is LDNA
  modified_base 23: mod_base = OTHER; note = The residue in this position is LDNA
  modified_base 26: mod_base = OTHER; note = The residue in this position is LDNA
  modified_base 28..30: mod_base = OTHER; note = The residue at this position has a 3-phosphorothioate.
  modified_base 29..31: mod_base = OTHER; note = The residue at this position is a 2-O-methylated ribonucleotide.

SEQ ID NO: 193 - RNA, 31 nt, synthetic construct; identical to SEQ ID NO: 42
  source 1..31: mol_type = other RNA
  modified_base 1: mod_base = OTHER; note = The residue in this position is DNA
  modified_base 3: mod_base = OTHER; note = The residue in this position is DNA
  modified_base 5: mod_base = OTHER; note = The residue in this position is DNA
  modified_base 7: mod_base = OTHER; note = The residue in this position is DNA
  modified_base 9: mod_base = OTHER; note = The residue in this position is DNA
  modified_base 11: mod_base = OTHER; note = The residue in this position is DNA
  modified_base 13: mod_base = OTHER; note = The residue in this position is DNA
  modified_base 15: mod_base = OTHER; note = The residue in this position is DNA
  modified_base 17: mod_base = OTHER; note = The residue in this position is DNA
  modified_base 19: mod_base = OTHER; note = The residue in this position is DNA
  modified_base 21: mod_base = OTHER; note = The residue in this position is DNA
  modified_base 23: mod_base = OTHER; note = The residue in this position is DNA
  modified_base 25: mod_base = OTHER; note = The residue in this position is DNA
  modified_base 27: mod_base = OTHER; note = The residue in this position is DNA
  modified_base 2: mod_base = OTHER; note = The residue at this position is a 2-O-methylated ribonucleotide.
  modified_base 4: mod_base = OTHER; note = The residue at this position is a 2-O-methylated ribonucleotide.
  modified_base 6: mod_base = OTHER; note = The residue at this position is a 2-O-methylated ribonucleotide.
  modified_base 8: mod_base = OTHER; note = The residue at this position is a 2-O-methylated ribonucleotide.
  modified_base 10: mod_base = OTHER; note = The residue at this position is a 2-O-methylated ribonucleotide.
  modified_base 12: mod_base = OTHER; note = The residue at this position is a 2-O-methylated ribonucleotide.
  modified_base 14: mod_base = OTHER; note = The residue at this position is a 2-O-methylated ribonucleotide.
  modified_base 16: mod_base = OTHER; note = The residue at this position is a 2-O-methylated ribonucleotide.
  modified_base 18: mod_base = OTHER; note = The residue at this position is a 2-O-methylated ribonucleotide.
  modified_base 20: mod_base = OTHER; note = The residue at this position is a 2-O-methylated ribonucleotide.
  modified_base 22: mod_base = OTHER; note = The residue at this position is a 2-O-methylated ribonucleotide.
  modified_base 24: mod_base = OTHER; note = The residue at this position is a 2-O-methylated ribonucleotide.
  modified_base 26: mod_base = OTHER; note = The residue at this position is a 2-O-methylated ribonucleotide.
  modified_base 28..31: mod_base = OTHER; note = The residue at this position is a 2-O-methylated ribonucleotide.

SEQ ID NO: 194 - RNA, 31 nt, synthetic construct; identical to SEQ ID NO: 42
  source 1..31: mol_type = other RNA
  modified_base 2: mod_base = OTHER; note = The residue in this position is DNA
  modified_base 4: mod_base = OTHER; note = The residue in this position is DNA
  modified_base 6: mod_base = OTHER; note = The residue in this position is DNA
  modified_base 8: mod_base = OTHER; note = The residue in this position is DNA
  modified_base 10: mod_base = OTHER; note = The residue in this position is DNA
  modified_base 12: mod_base = OTHER; note = The residue in this position is DNA
  modified_base 14: mod_base = OTHER; note = The residue in this position is DNA
  modified_base 16: mod_base = OTHER; note = The residue in this position is DNA
  modified_base 18: mod_base = OTHER; note = The residue in this position is DNA
  modified_base 20: mod_base = OTHER; note = The residue in this position is DNA
  modified_base 22: mod_base = OTHER; note = The residue in this position is DNA
  modified_base 24: mod_base = OTHER; note = The residue in this position is DNA
  modified_base 26: mod_base = OTHER; note = The residue in this position is DNA
  modified_base 28: mod_base = OTHER; note = The residue in this position is DNA
  modified_base 1: mod_base = OTHER; note = The residue in this position has a 2-Fluoro modification
  modified_base 3: mod_base = OTHER; note = The residue in this position has a 2-Fluoro modification
  modified_base 5: mod_base = OTHER; note = The residue in this position has a 2-Fluoro modification
  modified_base 7: mod_base = OTHER; note = The residue in this position has a 2-Fluoro modification
  modified_base 9: mod_base = OTHER; note = The residue in this position has a 2-Fluoro modification
  modified_base 11: mod_base = OTHER; note = The residue in this position has a 2-Fluoro modification
  modified_base 13: mod_base = OTHER; note = The residue in this position has a 2-Fluoro modification
  modified_base 15: mod_base = OTHER; note = The residue in this position has a 2-Fluoro modification
  modified_base 17: mod_base = OTHER; note = The residue in this position has a 2-Fluoro modification
  modified_base 19: mod_base = OTHER; note = The residue in this position has a 2-Fluoro modification
  modified_base 21: mod_base = OTHER; note = The residue in this position has a 2-Fluoro modification
  modified_base 23: mod_base = OTHER; note = The residue in this position has a 2-Fluoro modification
  modified_base 25: mod_base = OTHER; note = The residue in this position has a 2-Fluoro modification
  modified_base 27: mod_base = OTHER; note = The residue in this position has a 2-Fluoro modification
  modified_base 29..31: mod_base = OTHER; note = The residue at this position is a 2-O-methylated ribonucleotide.
  modified_base 28..30: mod_base = OTHER; note = The residue at this position has a 3-phosphorothioate.

SEQ ID NO: 195 - RNA, 31 nt, synthetic construct; identical to SEQ ID NO: 42
  source 1..31: mol_type = other RNA
  modified_base 1..20: mod_base = OTHER; note = The residue in this position is DNA
  modified_base 21: mod_base = OTHER; note = The residue at this position is a 2-O-methylated ribonucleotide.
  modified_base 23: mod_base = OTHER; note = The residue at this position is a 2-O-methylated ribonucleotide.
  modified_base 25: mod_base = OTHER; note = The residue at this position is a 2-O-methylated ribonucleotide.
  modified_base 27: mod_base = OTHER; note = The residue at this position is a 2-O-methylated ribonucleotide.
  modified_base 29..31: mod_base = OTHER; note = The residue at this position is a 2-O-methylated ribonucleotide.
  modified_base 28..30: mod_base = OTHER; note = The residue at this position has a 3-phosphorothioate.

SEQ ID NO: 196 - RNA, 31 nt, synthetic construct; identical to SEQ ID NO: 42
  source 1..31: mol_type = other RNA
  modified_base 22: mod_base = OTHER; note = The residue at this position has a 3-phosphorothioate.
  modified_base 24: mod_base = OTHER; note = The residue at this position has a 3-phosphorothioate.
  modified_base 26: mod_base = OTHER; note = The residue at this position has a 3-phosphorothioate.
  modified_base 28..30: mod_base = OTHER; note = The residue at this position has a 3-phosphorothioate.
  modified_base 29..31: mod_base = OTHER; note = The residue at this position is a 2-O-methylated ribonucleotide.

SEQ ID NO: 197 - DNA, 14 bp, synthetic construct
  source 1..14: mol_type = other DNA
gctgcagaagggat

SEQ ID NO: 198 - RNA, 14 nt, synthetic construct
  source 1..14: mol_type = other RNA
  modified_base 1..11: mod_base = OTHER; note = The residue in this position is DNA
  modified_base 11..13: mod_base = OTHER; note = The residue at this position has a 3-phosphorothioate.
gctgcagaagggat

SEQ ID NO: 199 - RNA, 14 nt, synthetic construct
  source 1..14: mol_type = other RNA
  modified_base 1..11: mod_base = OTHER; note = The residue in this position is DNA
  modified_base 14: mod_base = OTHER; note = The residue in this position is DNA
  modified_base 11..13: mod_base = OTHER; note = The residue at this position has a 3-phosphorothioate.
gctgcagaagggat

SEQ ID NO: 200 - RNA, 31 nt, synthetic construct; identical to SEQ ID NO: 42
  source 1..31: mol_type = other RNA
  modified_base 1..17: mod_base = OTHER; note = The residue in this position is DNA
  modified_base 20: mod_base = OTHER; note = The residue in this position is LDNA
  modified_base 23: mod_base = OTHER; note = The residue in this position is LDNA
  modified_base 26: mod_base = OTHER; note = The residue in this position is LDNA
  modified_base 28..30: mod_base = OTHER; note = The residue at this position has a 3-phosphorothioate.
  modified_base 29..31: mod_base = OTHER; note = The residue at this position is a 2-O-methylated ribonucleotide.

SEQ ID NO: 201 - RNA, 31 nt, synthetic construct; identical to SEQ ID NO: 42
  source 1..31: mol_type = other RNA
  modified_base 1: mod_base = OTHER; note = The residue in this position is DNA
  modified_base 3: mod_base = OTHER; note = The residue in this position is DNA
  modified_base 5: mod_base = OTHER; note = The residue in this position is DNA
  modified_base 7: mod_base = OTHER; note = The residue in this position is DNA
  modified_base 9: mod_base = OTHER; note = The residue in this position is DNA
  modified_base 11: mod_base = OTHER; note = The residue in this position is DNA
  modified_base 13: mod_base = OTHER; note = The residue in this position is DNA
  modified_base 15: mod_base = OTHER; note = The residue in this position is DNA
  modified_base 17: mod_base = OTHER; note = The residue in this position is DNA
  modified_base 19: mod_base = OTHER; note = The residue in this position is DNA
  modified_base 25: mod_base = OTHER; note = The residue in this position is DNA
  modified_base 23: mod_base = OTHER; note = The residue in this position is DNA
  modified_base 21: mod_base = OTHER; note = The residue in this position is DNA
  modified_base 27: mod_base = OTHER; note = The residue in this position is DNA
  modified_base 2: mod_base = OTHER; note = The residue at this position is a 2-O-methylated ribonucleotide.
  modified_base 4: mod_base = OTHER; note = The residue at this position is a 2-O-methylated ribonucleotide.
  modified_base 6: mod_base = OTHER; note = The residue at this position is a 2-O-methylated ribonucleotide.
  modified_base 8: mod_base = OTHER; note = The residue at this position is a 2-O-methylated ribonucleotide.
  modified_base 10: mod_base = OTHER; note = The residue at this position is a 2-O-methylated ribonucleotide.
  modified_base 12: mod_base = OTHER; note = The residue at this position is a 2-O-methylated ribonucleotide.
  modified_base 14: mod_base = OTHER; note = The residue at this position is a 2-O-methylated ribonucleotide.
  modified_base 16: mod_base = OTHER; note = The residue at this position is a 2-O-methylated ribonucleotide.
  modified_base 18: mod_base = OTHER; note = The residue at this position is a 2-O-methylated ribonucleotide.
  modified_base 20: mod_base = OTHER; note = The residue at this position is a 2-O-methylated ribonucleotide.
  modified_base 28..31: mod_base = OTHER; note = The residue at this position is a 2-O-methylated ribonucleotide.
  modified_base 28..30: mod_base = OTHER; note = The residue at this position has a 3-phosphorothioate.

SEQ ID NO: 202 - RNA, 31 nt, synthetic construct; identical to SEQ ID NO: 42
  source 1..31: mol_type = other RNA
  modified_base 2: mod_base = OTHER; note = The residue in this position is DNA
  modified_base 4: mod_base = OTHER; note = The residue in this position is DNA
  modified_base 6: mod_base = OTHER; note = The residue in this position is DNA
  modified_base 8: mod_base = OTHER; note = The residue in this position is DNA
  modified_base 10: mod_base = OTHER; note = The residue in this position is DNA
  modified_base 12: mod_base = OTHER; note = The residue in this position is DNA
  modified_base 14: mod_base = OTHER; note = The residue in this position is DNA
  modified_base 16: mod_base = OTHER; note = The residue in this position is DNA
  modified_base 18: mod_base = OTHER; note = The residue in this position is DNA
  modified_base 20: mod_base = OTHER; note = The residue in this position is DNA
  modified_base 28: mod_base = OTHER; note = The residue in this position is DNA
  modified_base 24: mod_base = OTHER; note = The residue in this position is DNA
  modified_base 22: mod_base = OTHER; note = The residue in this position is DNA
  modified_base 26: mod_base = OTHER; note = The residue in this position is DNA
  modified_base 1: mod_base = OTHER; note = The residue in this position has a 2-Fluoro modification
  modified_base 3: mod_base = OTHER; note = The residue in this position has a 2-Fluoro modification
  modified_base 5: mod_base = OTHER; note = The residue in this position has a 2-Fluoro modification
  modified_base 7: mod_base = OTHER; note = The residue in this position has a 2-Fluoro modification
  modified_base 9: mod_base = OTHER; note = The residue in this position has a 2-Fluoro modification
  modified_base 11: mod_base = OTHER; note = The residue in this position has a 2-Fluoro modification
  modified_base 13: mod_base = OTHER; note = The residue in this position has a 2-Fluoro modification
  modified_base 15: mod_base = OTHER; note = The residue in this position has a 2-Fluoro modification
  modified_base 17: mod_base = OTHER; note = The residue in this position has a 2-Fluoro modification
  modified_base 19: mod_base = OTHER; note = The residue in this position has a 2-Fluoro modification
  modified_base 21: mod_base = OTHER; note = The residue in this position has a 2-Fluoro modification
  modified_base 23: mod_base = OTHER; note = The residue in this position has a 2-Fluoro modification
  modified_base 25: mod_base = OTHER; note = The residue in this position has a 2-Fluoro modification
  modified_base 27: mod_base = OTHER; note = The residue in this position has a 2-Fluoro modification
  modified_base 29..31: mod_base = OTHER; note = The residue at this position is a 2-O-methylated ribonucleotide.
  modified_base 28..30: mod_base = OTHER; note = The residue at this position has a 3-phosphorothioate.

SEQ ID NO: 203 - RNA, 31 nt, synthetic construct; identical to SEQ ID NO: 42
  source 1..31: mol_type = other RNA
  modified_base 1..20: mod_base = OTHER; note = The residue in this position is DNA
  modified_base 21: mod_base = OTHER; note = The residue at this position is a 2-O-methylated ribonucleotide.
  modified_base 23: mod_base = OTHER; note = The residue at this position is a 2-O-methylated ribonucleotide.
  modified_base 25: mod_base = OTHER; note = The residue at this position is a 2-O-methylated ribonucleotide.
  modified_base 27: mod_base = OTHER; note = The residue at this position is a 2-O-methylated ribonucleotide.
  modified_base 29..31: mod_base = OTHER; note = The residue at this position is a 2-O-methylated ribonucleotide.
  modified_base 28..30: mod_base = OTHER; note = The residue at this position has a 3-phosphorothioate.

SEQ ID NO: 204 - RNA, 31 nt, synthetic construct; identical to SEQ ID NO: 42
  source 1..31: mol_type = other RNA
  modified_base 1..20: mod_base = OTHER; note = The residue in this position is DNA
  modified_base 24: mod_base = OTHER; note = The residue at this position has a 3-phosphorothioate.
  modified_base 22: mod_base = OTHER; note = The residue at this position has a 3-phosphorothioate.
  modified_base 26: mod_base = OTHER; note = The residue at this position has a 3-phosphorothioate.
  modified_base 28..30: mod_base = OTHER; note = The residue at this position has a 3-phosphorothioate.
  modified_base 29..31: mod_base = OTHER; note = The residue at this position is a 2-O-methylated ribonucleotide.

SEQ ID NO: 205 - DNA, 14 bp, synthetic construct
  source 1..14: mol_type = other DNA
gctgcagaagggat

SEQ ID NO: 206 - RNA, 14 nt, synthetic construct
  source 1..14: mol_type = other RNA
  modified_base 12..14: mod_base = OTHER; note = The residue at this position is a 2-O-methylated ribonucleotide.
  modified_base 11..13: mod_base = OTHER; note = The residue at this position has a 3-phosphorothioate.
gctgcagaagggat

SEQ ID NO: 207 - RNA, 14 nt, synthetic construct
  source 1..14: mol_type = other RNA
  modified_base 1..11: mod_base = OTHER; note = The residue in this position is DNA
  modified_base 12..14: mod_base = OTHER; note = The residue at this position is a 2-O-methylated ribonucleotide.
  modified_base 11..13: mod_base = OTHER; note = The residue at this position has a 3-phosphorothioate.
gctgcagaagggat

SEQ ID NO: 208 - DNA, 31 bp, synthetic construct; identical to SEQ ID NO: 43
  source 1..31: mol_type = other DNA

SEQ ID NO: 209 - RNA, 31 nt, synthetic construct; identical to SEQ ID NO: 42
  source 1..31: mol_type = other RNA
  modified_base 1..17: mod_base = OTHER; note = The residue in this position is DNA
  modified_base 28..30: mod_base = OTHER; note = The residue at this position has a 3-phosphorothioate.

SEQ ID NO: 210 - RNA, 31 nt, synthetic construct; identical to SEQ ID NO: 42
  source 1..31: mol_type = other RNA
  modified_base 1..25: mod_base = OTHER; note = The residue in this position is DNA
  modified_base 28..30: mod_base = OTHER; note = The residue at this position has a 3-phosphorothioate.

SEQ ID NO: 211 - DNA, 31 bp, synthetic construct; identical to SEQ ID NO: 43
  source 1..31: mol_type = other DNA
  modified_base 27..30: mod_base = OTHER; note = The residue at this position has a 3-phosphorothioate.

SEQ ID NO: 212 - DNA, 31 bp, synthetic construct; identical to SEQ ID NO: 43
  source 1..31: mol_type = other DNA
  modified_base 25..30: mod_base = OTHER; note = The residue at this position has a 3-phosphorothioate.

SEQ ID NO: 213 - DNA, 31 bp, synthetic construct; identical to SEQ ID NO: 43
  source 1..31: mol_type = other DNA
  modified_base 23..30: mod_base = OTHER; note = The residue at this position has a 3-phosphorothioate.

SEQ ID NO: 214 - DNA, 31 bp, synthetic construct; identical to SEQ ID NO: 43
  source 1..31: mol_type = other DNA
  modified_base 21..30: mod_base = OTHER; note = The residue at this position has a 3-phosphorothioate.

SEQ ID NO: 215 - DNA, 2580 bp, synthetic construct
  source 1..2580: mol_type = other DNA
gcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctactctggaggatctagcggaggatcctctggcagcgagacaccaggaacaagcgagtcagcaacaccagagagcagtggcggcagcagcggcggcagcagcaccctaaatatagaagatgagtatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttaatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggaaggcaggcttctgtcgcctcttcatccctgggtttgcagaaatggcagcccccctgtaccctctcaccaaaccggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggtggctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctctggcggctcaaaaagaaccgccgacggcagcgaattcgagcccaagaagaagaggaaagtc

SEQ ID NO: 216 - DNA, 2634 bp, synthetic construct
  source 1..2634: mol_type = other DNA
gccgccccaaagaagaagcggaaggtctatccatatgatgttccagattatgctggggcccagccggccagatctatggcggaagcgggtatcaccggcacgtggtacaaccagtctggttctaccttcaccgttaccgcgggtgcggacggtaacctgaccggtcagtacgaaaaccgtgcgcagggcactggttgccagaactctccgtacaccctgaccggtcgttacaacggtaccaaactggaatggcgtgttgaatggaacaactctaccgaaaactgccactctcgtaccgaatggcgtggtcagtaccagggtggtgcggaagcgcgtatcaacacccagtggaacctgacctacgaaggtggttctggtccggcgaccgaacagggtcaggacaccttcaccaaagttaaaccgtctgcggcgtctggatcctctggaggatctagcggaggatcctctggcagcgagacaccaggaacaagcgagtcagcaacaccagagagcagtggcggcagcagcggcggcagcagcaccctaaatatagaagatgagtatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttaatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggaaggcaggcttctgtcgcctcttcatccctgggtttgcagaaatggcagcccccctgtaccctctcaccaaaccggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggtggctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctctggcggctcaaaaagaaccgccgacggcagcgaattcgagcccaagaagaagaggaaagtc

SEQ ID NO: 217 - DNA, 2229 bp, synthetic construct
  source 1..2229: mol_type = other DNA
gcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctactctggaggatctagcggaggatcctctggcagcgagacaccaggaacaagcgagtcagcaacaccagagagcagtggcggcagcagcggcggcagcagcaagcatatgcccagaaaaatgtattcctgtgattttgagacgacaaccaaagtagaagattgtcgggtttgggcctacggatacatgaacattgaagaccattcagaatataaaatcggtaacagtctcgatgaattcatggcatgggtgttgaaagtccaagccgatctttatttccataacttgaaatttgacggcgcttttatcattaactggcttgagcgcaatggcttcaagtggtcagcggatgggcttccaaacacgtataatacgattatatcccgcatgggtcagtggtacatgatcgacatatgtcttggttacaaaggcaaacgcaagatccataccgttatctatgacagtttgaagaagttgccgtttccagttaagaaaattgcgaaagactttaaacttacggtgcttaagggcgacatcgactaccataaggaacggccagttggttacaagataacgcctgaggagtacgcttatatcaagaatgacatacagattatagctgaagcattgctcatacagttcaaacaaggcctggataggatgaccgctggcagtgattcacttaaaggatttaaagacataattactacgaagaagtttaagaaagtgtttccaaccctcagtctgggcctcgacaaagaagtgcgatatgcgtaccgcggcgggttcacatggcttaatgaccgattcaaggaaaaggaaataggagaagggatggttttcgatgttaatagcctctatcccgcgcaaatgtacagtaggcttttgccatatggcgaaccaatcgtgttcgaggggaagtatgtatgggatgaggactatcctctccacattcagcacatccggtgtgagttcgagcttaaagagggatatatccctactatccagataaaacgctctcgattttacaaggggaacgaataccttaagtcatccgggggggaaatcgcagatttgtggttgtcaaatgttgaccttgagttgatgaaggaacactatgatctctacaacgtcgaatacatttcaggtttgaaattcaaggcaaccaccgggctttttaaagatttcatcgataaatggacctacataaaaacgacctcagaaggagccataaagcagttggctaaacttatgcttaattctctctacggaaaatttgcaagtaatcctgacgttacagggaaagtcccgtacttgaaagaaaatggagcgctggggtttaggctcggtgaggaagaaaccaaggatccagtctacacaccaatgggcgtatttatcactgcatgggcgcgatatacaacaatcacggccgctcaggcctgttacgatcgcataatttactgcgacaccgattcaatccaccttaccggaacagagatcccggacgttattaaagatattgtggacccaaaaaagctgggctactgggcccacgagtccaccttcaagcgcgccaagtatctgcggcaaaaaacttacatccaggatatatacatgaaggaggtagacggtaagcttgtcgaaggctcaccggatgactataccgatatcaaattctcagtaaagtgtgctggtatgactgacaagataaagaaggaagttaccttcgagaacttcaaggtagggttcagtagaaaaatgaaacccaagccagtgcaagtgcctggtggagtagtactggtggacgatacattcacgatcaaacccaagaagaagaggaaagtc

SEQ ID NO: 218 - DNA, 3288 bp, synthetic construct
  source 1..3288: mol_type = other DNA
gcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctactctggaggatctagcggaggatcctctggcagcgagacaccaggaacaagcgagtcagcaacaccagagagcagtggcggcagcagcggcggcagcagcgttcagattcctcaaaacccgttgatcctcgttgatggctccagctacctgtatagggcatatcacgcatttcccccattgacaaattctgccggtgaaccgaccggagctatgtatggagtgctgaacatgctgcgctcactgatcatgcaatataaaccgacacacgcagctgtggtattcgacgccaaaggaaaaacgttcagagatgaactgttcgaacactataagtcccatcggccgccaatgcccgatgatcttcgcgctcaaattgaaccgttgcatgcaatggttaaggctatggggctgcctcttttggctgtatctggggtggaggcagatgatgtaatcggtaccttggcaagggaagccgagaaagccgggagaccagtcttgatctctacgggagacaaagacatggcccagctcgtaactcccaacattactcttattaacacaatgactaacacgatactgggacctgaagaagttgtcaataagtacggggtccctcctgagcttattatagactttttggccctcatgggtgacagcagtgacaatataccaggggttccaggcgttggagagaaaactgcccaagccttgctgcagggcctggggggtcttgacacgctctacgcggaaccggaaaaaatcgcaggtctcagctttcgaggggctaagaccatggctgctaaactcgagcagaataaagaagtagcttatctttcctatcagttggccacgattaaaacggacgtcgaattggaactcacttgtgaacaacttgaagtacaacaaccggcggcagaggaattgctcggcctctttaaaaagtatgagttcaagcgctggactgccgacgtcgaagctggaaagtggctgcaagcgaagggagcgaaaccggctgctaagccgcaggaaacctctgtagcagacgaggcaccagaggtgacggctactgttatttcctacgacaattacgtcacaattctcgatgaggaaacccttaaagcgtggatcgctaaacttgaaaaagcccccgttttcgcttttgacaccgagactgacagcctggataacatatctgcgaatttggtgggtctgtcctttgcaatcgagcccggagttgctgcatacatacctgttgcgcacgattacctcgatgcgccagatcaaatatctcgggaaagagccctggaactgctgaagccacttttggaggacgagaaggccctcaaagtgggtcagaaccttaagtacgcaagaggcattctggcgaactatgggattgaactccgcggaatagctttcgacacaatgcttgagtcatatattctcaactcagttgccggaaggcatgatatggatagtttggctgaaagatggttgaaacataaaactatcacattcgaagagattgcggggaagggaaagaaccaacttacgttcaaccagatcgctctggaggaagctggtagatacgctgctgaagacgcggatgtaacgctccagctccacttgaagatgtggccagacttgcaaaagcacaagggccctctcaacgtcttcgaaaatatcgaaatgcctctcgtccccgtcctgtcaagaattgagcgaaatggggtgaagatcgacccgaaggtgctgcacaaccatagtgaagagctcactcttagactcgctgaattggaaaaaaaggcgcacgagatcgcgggcgaggaatttaacctgagttccacgaaacaacttcagacaattctctttgaaaagcaggggatcaaaccactgaagaaaactccgggcggggccccctccacgtctgaggaagtgctggaagaactggcattggattaccctctgccgaaggttatacttgagtaccgcggccttgcgaaactcaagagtacatatacggacaagctgcccctgatgatcaacccgaagaccggtcgggtgcacacaagctatcatcaggctgtgacggcgacaggtaggttgtcttccaccgacccgaatctgcaaaatataccggtgcggaacgaggaagggagaaggataagacaagccttcattgccccagaagactacgtaattgtctcagcggattattcacaaaatgaactccggattatggcgcaccttagtcgagacaagggattgctcactgctttcgcggaaggcaaggacatacatcgggcaactgctgcagaagtatacgggcttccactcgaaacggttaccagtgagcagcgacggagtgcgaagcggatcaattttggccttatttacggtatgtcagccttcggactggccaggcagcttaatataccccggaaggaggcgcagaagtatatggacttgtatttcgagcggtatcatggcgttctggaatacatggagaggactcgggcccaggcaaaagaacaggggtacgttgaaactctcgatggaagaaggctgtacctcccggacattaagtctagtaacggagcccgaagagcggcggccgaaagagcagccataaatgcaccaatgcaaggcacagcggccgatataatcaagagagcgatgatcgctgtcgacgcatggttgcaagctgaacagccaagggtaaggatgatcatgcaagtacacgatgaacttgtgttcgaggttcacaaagatgatgtggacgcagttgccaaacagatacatcagctcatggagaattgtaccaggctggacgtgcctctgctcgttgaagtcggctccggagaaaactgggatcaggcacatcccaagaagaagaggaaagtc

SEQ ID NO: 219 - DNA, 2271 bp, synthetic construct
  source 1..2271: mol_type = other DNA
gccccaaagaagaagcggaaggtctatccatatgatgttccagattatgctggggcccagccggccagatctatggcggaagcgggtatcaccggcacgtggtacaaccagtctggttctaccttcaccgttaccgcgggtgcggacggtaacctgaccggtcagtacgaaaaccgtgcgcagggcactggttgccagaactctccgtacaccctgaccggtcgttacaacggtaccaaactggaatggcgtgttgaatggaacaactctaccgaaaactgccactctcgtaccgaatggcgtggtcagtaccagggtggtgcggaagcgcgtatcaacacccagtggaacctgacctacgaaggtggttctggtccggcgaccgaacagggtcaggacaccttcaccaaagttaaaccgtctgcggcgtctggatccagcgctggaggaggtggaagcggaggaggaggaagcggaggaggaggtagcggacctaagaaaaagaggaaggtggcggccgctggatccaagcatatgcccagaaaaatgtattcctgtgattttgagacgacaaccaaagtagaagattgtcgggtttgggcctacggatacatgaacattgaagaccattcagaatataaaatcggtaacagtctcgatgaattcatggcatgggtgttgaaagtccaagccgatctttatttccataacttgaaatttgacggcgcttttatcattaactggcttgagcgcaatggcttcaagtggtcagcggatgggcttccaaacacgtataatacgattatatcccgcatgggtcagtggtacatgatcgacatatgtcttggttacaaaggcaaacgcaagatccataccgttatctatgacagtttgaagaagttgccgtttccagttaagaaaattgcgaaagactttaaacttacggtgcttaagggcgacatcgactaccataaggaacggccagttggttacaagataacgcctgaggagtacgcttatatcaagaatgacatacagattatagctgaagcattgctcatacagttcaaacaaggcctggataggatgaccgctggcagtgattcacttaaaggatttaaagacataattactacgaagaagtttaagaaagtgtttccaaccctcagtctgggcctcgacaaagaagtgcgatatgcgtaccgcggcgggttcacatggcttaatgaccgattcaaggaaaaggaaataggagaagggatggttttcgatgttaatagcctctatcccgcgcaaatgtacagtaggcttttgccatatggcgaaccaatcgtgttcgaggggaagtatgtatgggatgaggactatcctctccacattcagcacatccggtgtgagttcgagcttaaagagggatatatccctactatccagataaaacgctctcgattttacaaggggaacgaataccttaagtcatccgggggggaaatcgcagatttgtggttgtcaaatgttgaccttgagttgatgaaggaacactatgatctctacaacgtcgaatacatttcaggtttgaaattcaaggcaaccaccgggctttttaaagatttcatcgataaatggacctacataaaaacgacctcagaaggagccataaagcagttggctaaacttatgcttaattctctctacggaaaatttgcaagtaatcctgacgttacagggaaagtcccgtacttgaaagaaaatggagcgctggggtttaggctcggtgaggaagaaaccaaggatccagtctacacaccaatgggcgtatttatcactgcatgggcgcgatatacaacaatcacggccgctcaggcctgttacgatcgcataatttactgcgacaccgattcaatccaccttaccggaacagagatcccggacgttattaaagatattgtggacccaaaaaagctgggctactgggcccacgagtccaccttcaagcgcgccaagtatctgcggcaaaaaacttacatccaggatatatacatgaaggaggtagacggtaagcttgtcgaaggctcaccggatgactataccgatatcaaattctcagtaaagtgtgctggtatgactgacaagataaagaaggaagttaccttcgagaacttcaaggtagggttcagtagaaaaatgaaacccaagccagtgcaagtgcctggtggagtagtactggtggacgatacattcacgatcaaacccaagaagaagaggaaagtc